(19) United States
(12) Patent Application Publication
(10) Pub. No.: US 2009/0105239 A1
(43) Pub. Date: Apr. 23, 2009

(54) 2-PYRIDINE DERIVATIVES AS INHIBITORS OF NEUTROPHILE ELASTASE

(76) Inventors: Thomas Brimert, Lund (SE); Karolina Lawitz, Lund (SE); Hans Lonn, Lund (SE); Antonios Nikitidis, Lund (SE); Asim Kumar Ray, Lund (SE); Jenny Sandmark, Molndal (SE)

Correspondence Address: MORGAN LEWIS & BOCKIUS LLP, 1111 PENNSYLVANIA AVENUE NW, WASHINGTON, DC, 20004, US

(21) Appl. No.: 11/908,748

(22) PCT Filed: Mar. 14, 2006
(86) PCT No.: PCT/SE2006/000328

§ 371 Date: May 20, 2008

(30) Foreign Application Priority Data
Mar. 16, 2005 (SE) 0500604-4

(51) Int. Cl. A61K 31/5377; C07D 213/63; A61K 31/44; A61K 31/4439; C07D 413/02; C07D 401/02; A61K 31/444

(52) U.S. Cl. 514/231.5; 546/291; 514/346; 514/336; 546/276.4; 546/255; 514/332; 544/124

(57) ABSTRACT

The invention provides compounds of formula wherein R¹, R³, R⁴, R⁵, R⁶, R¹⁴, X, W and Z are as defined in the specification and optical isomers, racemates and tautomers thereof, and pharmaceutically acceptable salts thereof; together with processes for their preparation, pharmaceutical compositions containing them and their use in therapy. The compounds are inhibitors of human neutrophil elastase.

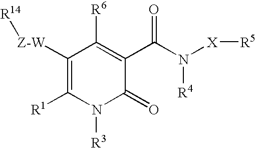

### FIELD OF THE INVENTION

The present invention relates to 2-pyridone derivatives, processes for their preparation, pharmaceutical compositions containing them and their use in therapy.

### BACKGROUND OF THE INVENTION

Elastases are possibly the most destructive enzymes in the body, having the ability to degrade virtually all connective tissue components. The uncontrolled proteolytic degradation by elastases has been implicated in a number of pathological conditions. Human neutrophil elastase (hNE), a member of the chymotrypsin superfamily of serine proteases is a 33-KDa enzyme stored in the azurophilic granules of the neutrophils. In neutrophils the concentration of NE exceeded 5 mM and its total cellular amount has been estimated to be up to 3 pg. Upon activation, NE is rapidly released from the granules into the extracellular space with some portion remaining bound to neutrophil plasma membrane (See Kawabat et al. 2002, Eur. J. Pharmacol. 451, 1-10). The main intracellular physiological function of NE is degradation of foreign organic molecules phagocytosed by neutrophils, whereas the main target for extracellular elastase is elastin (Janoff and Scherer, 1968, J. Exp. Med. 128, 1137-1155). NE is unique, as compared to other proteases (for example, proteinase 3) in that it has the ability to degrade almost all extracellular matrix and key plasma proteins (See Kawabat et al., 2002, Eur. J. Pharmacol. 451, 1-10). It degrades a wide range of extracellular matrix proteins such as elastin, Type 3 and type 4 collagens, laminin, fibronectin, cytokines, etc. (Ohbayashi, H., 2002, Expert Opin. Investig. Drugs, 11, 965-980). NE is a major common mediator of many pathological changes seen in chronic lung disease including epithelial damage (Stockley, R. A. 1994, Am. J. Resp. Crit. Care Med. 150, 109-113).

The destructive role of NE was solidified almost 40 years ago when Laurell and Eriksson reported an association of chronic airflow obstruction and emphysema with deficiency of serum α₁-antitrypsin (Laurell and Eriksson, 1963, Scand. J. Clin. Invest. 15, 132-140). Subsequently it was determined that α₁-antitrypsin is the most important endogenous inhibitor of human NE. The imbalance between human NE and endogenous antiprotease is believed to cause excess human NE in pulmonary tissues which is considered as a major pathogenic factor in chronic obstructive pulmonary disease (COPD). The excessive human NE shows a prominent destructive profile and actively takes part in destroying the normal pulmonary structures, followed by the irreversible enlargement of the respiratory airspaces, as seen mainly in emphysema. There is an increase in neutrophil recruitment into the lungs which is associated with increased lung elastase burden and emphysema in α₁-proteinase inhibitor-deficient mice (Cavarra et al., 1996, Lab. Invest. 75, 273-280). Individuals with higher levels of the NE-α₁ protease inhibitor complex in bronchoalveolar lavage fluid show significantly accelerated decline in lung functions compared to those with lower levels (Betsuyalu et al. 2000, Respiration, 67, 261-267). Instillation of human NE via the trachea in rats causes lung haemorrhage, neutrophil accumulation during acute phase and emphysematous changes during chronic phase (Karaki et al., 2002, Am. J. Resp. Crit. Care Med., 166, 496-500). Studies have shown that the acute phase of pulmonary emphysema and pulmonary haemorrhage caused by NE in hamsters can be inhibited by pre-treatment with inhibitors of NE (Fujie et al., 1999, Inflamm. Res. 48, 160-167).

Neutrophil-predominant airway inflammation and mucus obstruction of the airways are major pathologic features of COPD, including cystic fibrosis and chronic bronchitis. NE impairs mucin production, leading to mucus obstruction of the airways. NE is reported to increase the expression of major respiratory mucin gene, MUC5AC (Fischer, B. M & Voynow, 2002, Am. J. Respir. Cell Biol., 26, 447-452). Aerosol administration of NE to guinea pigs produces extensive epithelial damage within 20 minutes of contact (Suzuki et al., 1996, Am. J. Resp. Crit. Care Med., 153, 1405-1411). Furthermore NE reduces the ciliary beat frequency of human respiratory epithelium in vitro (Smallman et al., 1984, Thorax, 39, 663-667) which is consistent with the reduced mucociliary clearance that is seen in COPD patients (Currie et al., 1984, Thorax, 42, 126-130). The instillation of NE into the airways leads to mucus gland hyperplasia in hamsters (Lucey et al., 1985, Am. Resp. Crit. Care Med., 132, 362-366). A role for NE is also implicated in mucus hypersecretion in asthma. In an allergen sensitised guinea pig acute asthma model an inhibitor of NE prevented goblet cell degranulation and mucus hypersecretion (Nadel et al., 1999, Eur. Resp. J., 13, 190-196). NE has been also shown to play a role in the pathogenesis of pulmonary fibrosis. NE: α₁-protenase inhibitor complex is increased in serum of patients with pulmonary fibrosis, which correlates with the clinical parameters in these patients (Yamanouchi et al., 1998, Eur. Resp. J. 11, 120-125). In a murine model of human pulmonary fibrosis, a NE inhibitor reduced bleomycin-induced pulmonary fibrosis (Taooka et al., 1997, Am. J. Resp. Crit. Care Med., 156, 260-265). Furthermore investigators have shown that NE deficient mice are resistant to bleomycin-induced pulmonary fibrosis (Dunsmore et al., 2001, Chest, 120, 35S-36S). Plasma NE level was found to be elevated in patients who progressed to ARDS implicating the importance of NE in early ARDS disease pathogenesis. (Donnelly et al., 1995, Am. J. Res. Crit. Care Med., 151, 428-1433). The antiproteases and NE complexed with antiprotease are increased in lung cancer area (Marchandise et al., 1989, Eur. Resp. J. 2, 623-629). Recent studies have shown that polymorphism in the promoter region of the NE gene are associated with lung cancer development (Taniguchi et al., 2002, Clin. Cancer Res., 8, 1115-1120.

Acute lung injury caused by endotoxin in experimental animals is associated with elevated levels of NE (Kawabata, et al., 1999, Am. J. Resp. Crit. Care, 161, 2013-2018). Acute lung inflammation caused by intratracheal injection of lipopolysaccharide in mice has been shown to elevate the NE activity in bronchoalveolar lavage fluid which is significantly inhibited by a NE inhibitor (Fujie et al., 1999, Eur. J. Pharmacol., 374, 117-125; Yasui, et al., 1995, Eur. Resp. J., 8, 1293-1299). NE also plays an important role in the neutrophil-induced increase of pulmonary microvascular permeability observed in a model of acute lung injury caused by tumour necrosis factor α (TNFα) and phorbol myristate acetate (PMA) in isolated perfused rabbit lungs (Miyazaki et al., 1998, Am. J. Respir. Crit. Care Med., 157, 89-94).

A role for NE has also been suggested in monocrotoline-induced pulmonary vascular wall thickening and cardiac hypertrophy (Molteni et al., 1989, Biochemical Pharmacol. 38, 2411-2419). Serine elastase inhibitor reverses the monocrotaline-induced pulmonary hypertension and remodelling in rat pulmonary arteries (Cowan et al., 2000, Nature Medicine, 6, 698-702). Recent studies have shown that serine elastase, that is, NE or vascular elastase are important in cigarette smoke-induced muscularisation of small pulmonary arteries in guinea pigs (Wright et al., 2002, Am. J. Respir. Crit. Care Med., 166, 954-960).

NE plays a key role in experimental cerebral ischemic damage (Shimakura et al., 2000, Brain Research, 858, 55-60), ischemia-reperfusion lung injury (Kishima et al., 1998, Ann. Thorac. Surg. 65, 913-918) and myocardial ischemia in rat heart (Tiefenbacher et al., 1997, Eur. J. Physiol., 433, 563-570). Human NE levels in plasma are significantly increased above normal in inflammatory bowel diseases, for example, Crohn's disease and ulcerative colitis (Adeyemi et al., 1985, Gut, 26, 1306-1311). In addition NE has also been assumed to be involved in the pathogenesis of rheumatoid arthritis (Adeyemi et al., 1986, Rheumatol. Int., 6, 57). The development of collagen induced arthritis in mice is suppressed by a NE inhibitor (Kakimoto et al., 1995, Cellular Immunol. 165, 26-32).

Thus, human NE is known as one of the most destructive serine proteases and has been implicated in a variety of inflammatory diseases. The important endogenous inhibitor of human NE is α₁-antitrypsin. The imbalance between human NE and antiprotease is believed to give rise to an excess of human NE resulting in uncontrolled tissue destruction. The protease/antiprotease balance may be upset by a decreased availability of α₁-antitrypsin either through inactivation by oxidants such as cigarette smoke, or as a result of genetic inability to produce sufficient serum levels. Human NE has been implicated in the promotion or exacerbation of a number of diseases such as pulmonary emphysema, pulmonary fibrosis, adult respiratory distress syndrome (ARDS), ischemia reperfusion injury, rheumatoid arthritis and pulmonary hypertension.

### DISCLOSURE OF THE INVENTION

In accordance with the present invention, there is therefore provided a compound of formula (I)

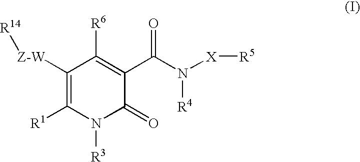

wherein

R¹ represents hydrogen or C₁-C₆ alkyl;

W represents S(O)ₘ wherein m represents an integer 0, 1 or 2;

Z represents a single bond, —CH₂— or NR²⁵—;

R¹⁴ represents a hydrogen atom or OH or a group selected from C₁-C₆ alkyl and a saturated or unsaturated 3- to 10-membered ring system optionally comprising at least one ring heteroatom selected from nitrogen, oxygen and sulphur; each group being optionally substituted with at least one substituent selected from phenyl, C₁-C₆ alkoxycarbonyl, halogen, C₁-C₄ alkyl C₁-C₄ alkoxy, CN, OH, NO₂, C₁-C₃ alkyl substituted by one or more F atoms, C₁-C₃ alkoxy substituted by one or more F atoms, NR¹²R¹³, C≡CR³⁰, CONR³¹R³², CHO, C₂-C₄ alkanoyl, S(O)ₚR³³ and OSO₂R³⁴;

R¹² and R¹³ independently represent H, C₁-C₆ alkyl, formyl or C₂-C₆ alkanoyl; or the group —NR¹²R¹³ together represents a 5 to 7 membered azacyclic ring optionally incorporating one further heteroatom selected from O, S and NR²⁶;

R³⁰ represents H, C₁-C₃ alkyl, Si(CH₃)₃ or phenyl;

R³³ and R³⁴ independently represent H or C₁-C₃ alkyl; said alkyl being optionally substituted by one or more F atoms;

R⁶ represents H or F;

R³ represents phenyl or a five- or six-membered heteroaromatic ring containing 1 to 3 heteroatoms independently selected from O, S and N; said ring being optionally substituted with at least one substituent selected from halogen, C₁-C₆ alkyl, cyano, C₁-C₆ alkoxy, nitro, methylcarbonyl, NR³⁵R³⁶, C₁-C₃ alkyl substituted by one or more F atoms or C₁-C₃ alkoxy substituted by one or more F atoms;

R³⁵ and R³⁶ independently represent H or C₁-C₃ alkyl; said allyl being optionally further substituted by one or more F atoms;

R⁴ represents hydrogen or C₁-C₆ alkyl optionally substituted with at least one substituent selected from fluoro, hydroxyl and C₁-C₆ alkoxy;

X represents a single bond, O, NR²⁴ or a group —C₁-C₆ alkylene-Y—, wherein Y represents a single bond, oxygen atom, NR²⁴ or S(O)_{w}; and said alkylene being optionally further substituted by OH, halogen, CN, NR³⁷R³⁸, C₁-C₃ alkoxy, CONR³⁹R⁴⁰, SO₂R⁴¹ and SO₂NR⁴²R⁴³;

or R⁴ and X are joined together such that the group —NR⁴X together represents a 5 to 7 membered azacyclic ring optionally incorporating one further heteroatom selected from O, S and NR⁴⁴; said ring being optionally substituted by C₁-C₆ alkyl or NR⁴⁵R⁴⁶; said alkyl being optionally further substituted by OH;

either R⁵ represents a monocyclic ring system selected from
i) phenoxy,
ii) phenyl,
iii) a 5- or 6-membered heteroaromatic ring comprising at least one ring heteroatom selected from nitrogen, oxygen and sulphur,
iv) a saturated or partially unsaturated C₃-C₆ hydrocarbyl ring, or
v) a saturated or partially unsaturated 4- to 7-membered heterocyclic ring comprising at least one ring heteroatom selected from oxygen, S(O)ᵣ and NR²⁰, wherein at least one of the ring carbon atoms may be optionally replaced by a carbonyl group,

or R⁵ represents a bicyclic ring system in which the two rings are independently selected from the monocyclic ring systems defined in ii), iii), iv) and v) above, wherein the two rings are either fused together, bonded directly to one another or are separated from one another by a linker group selected from oxygen, S(O)ₜ or C₁-C₆ alkylene optionally comprising one or more internal or terminal heteroatoms selected from oxygen, sulphur and NR²⁷ and being optionally substituted by at least one substituent selected from hydroxyl, oxo and C₁-C₆ alkoxy,

the monocyclic or bicyclic ring system being optionally substituted by at least one substituent selected from oxygen, CN, OH, C₁-C₆ alkyl, C₁-C₆ alkoxy, halogen, NR⁴⁷R⁴⁸, NO₂, OSO₂R⁴⁹, CO₂R⁵⁰, C(═NH)NH₂, C(O)NR⁵¹R⁵², C(S)NR⁵³R⁵⁴, SC(═NH)NH₂, NR⁵⁵C(═NH)NH₂, S(O)ᵥR²¹, SO₂NR⁵⁶R⁵⁷, C₁-C₃ alkoxy substituted by one or more F atoms and C₁-C₃ allyl substituted by SO₂R⁵⁸ or by one or more F atoms; said C₁-C₆ alkyl being optionally further substituted with at least one substituent selected from cyano, hydroxyl, C₁-C₆ alkoxy, C₁-C₆ alkylthio and —C(O)NR²²R²³;

or R⁵ may also represent H;

R²⁰ represents hydrogen, C₁-C₆ alkyl, C₁-C₆ alkylcarbonyl or C₁-C₆ alkoxycarbonyl;

R²¹ represents hydrogen, C₁-C₆ alkyl or C₃-C₈ cycloalkyl; said alkyl or cycloalkyl group being optionally further substituted by one or more substituents selected independently from OH, CN, C₁-C₃ alkoxy and CONR⁵⁹R⁶⁰;

R³⁷ and R³⁸ independently represent H, C₁-C₆ alkyl, formyl or C₂-C₆ alkanoyl;

R⁴⁷ and R⁴⁸ independently represent H, C₁-C₆ alkyl, formyl, C₂-C₆ alkanoyl, S(O)_{q}R⁶¹ or SO₂NR⁶²R⁶³; said alkyl group being optionally further substituted by halogen, CN, C₁-C₄ alkoxy or CONR⁶⁴R⁶⁵;

R⁴¹ and R⁶¹ independently represent H, C₁-C₆ alkyl or C₃-C₆ cycloalkyl;

p is 0, 1 or 2;

q is 0, 1 or 2;

r is 0, 1 or 2;

t is 0, 1 or 2;

w is 0, 1 or 2;

v is 0, 1 or 2;

R²², R²³, R²⁴, R²⁵, R²⁶, R²⁷, R³¹, R³², R³⁹, R⁴⁰, R⁴², R⁴³, R⁴⁴, R⁴⁵, R⁴⁶, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶², R⁶³, R⁶⁴ and R⁶⁵ each independently represent hydrogen or C₁-C₆ alkyl;

or a pharmaceutically acceptable salt thereof.

In the context of the present specification, unless otherwise stated, an alkyl, alkenyl or alkynyl substituent group or an alkyl moiety in a substituent group may be linear or branched. Similarly, an alkylene group may be linear or branched. In the definition of R¹⁴, the saturated or unsaturated 3- to 10-membered ring system may have alicyclic or aromatic properties. An unsaturated ring system will be partially or fully unsaturated.

R¹ represents hydrogen or C₁-C₆ alkyl (e.g. methyl, ethyl, n-propyl, isopropyl, n-butyl, isobutyl, tert-butyl, n-pentyl or n-hexyl).

In one embodiment of the invention, R¹ represents a C₁-C₄ or C₁-C₂ alkyl group, in particular a methyl group.

W represents a group S, S(O) or S(O)₂. In one embodiment of the invention, W represents a group S(O) or S(O)₂. In another embodiment, W represents S(O).

Z represents a single bond, —CH₂— or —NR²⁵—. In one embodiment of the invention, Z represents a single bond, —CH₂—, —NH— or —NCH₃—. In another embodiment, Z represents a single bond such that the group W is bonded directly to the group R¹⁴.

R¹⁴ represents H or OH or a group selected from
C₁-C₆ alkyl (e.g. methyl, ethyl, n-propyl, isopropyl, n-butyl, isobutyl, tert-butyl, n-pentyl or n-hexyl) and
a saturated or unsaturated 3- to 10-membered (e.g. 3-, 4- or 5- to 6-, 7-, 8-, 9- or 10-membered) ring system optionally comprising at least one ring heteroatom (e.g. one, two, three or four ring heteroatoms independently) selected from nitrogen, oxygen and sulphur,
   each group being optionally substituted with at least one (e.g. one, two, three or four) substituent independently selected from halogen (e.g. fluorine, chlorine, bromine or iodine), cyano, CHO, hydroxyl, phenyl, nitro, —S(O)ₚR³³, —C(O)NR³¹R³², C₁-C₄ alkyl (e.g. methyl, ethyl, n-propyl, isopropyl, n-butyl, isobutyl or tert-butyl), C₁-C₄ alkoxy (e.g. methoxy, ethoxy, n-propoxy, isopropoxy, n-butoxy, isobutoxy or tert-butoxy), C₂-C₄ alkanoyl (e.g. methylcarbonyl (acetyl), ethylcarbonyl, n-propylcarbonyl or isopropylcarbonyl), C₁-C₆ alkoxycarbonyl (e.g. methoxycarbonyl, ethoxycarbonyl, n-propoxycarbonyl, isopropoxycarbonyl, n-butoxycarbonyl, isobutoxycarbonyl, tert-butoxycarbonyl, n-pentoxycarbonyl or n-hexoxycarbonyl), C₁-C₃ alkyl substituted by one or more F atoms (e.g. CH₂F, CHF₂, CF₃, CH₂CH₂F, CH₂CF₃, CF₂CF₃, CH(CF₃)₂ and CH₂CH₂CF₃), C₁-C₃ alkoxy substituted by one or more F atoms (e.g. OCH₂F, OCHF₂, OCF₃, OCH₂CH₂F, OCH₂CF₃, OCF₂CF₃, OCH(CF₃)₂ and OCH₂CH₂CF₃), NR¹²R¹³, C═CR³⁰ and OSO₂R³⁴

Examples of saturated or unsaturated 3- to 10-membered ring systems that may be used, which may be monocyclic or polycyclic (e.g. bicyclic) in which the two or more rings are fused, include one or more (in any combination) of cyclopropyl, cyclobutyl, cyclopentyl, cyclohexyl, bicyclo[2.2.1]heptyl, cyclopentenyl, cyclohexenyl, phenyl, pyrrolidinyl, piperidinyl, piperazinyl, morpholinyl, thiomorpholinyl, diazabicyclo[2.2.1]hept-2-yl, naphthyl, benzofuranyl, benzothienyl, benzodioxolyl, quinolinyl, oxazolyl, 2,3-dihydrobenzofuranyl, tetrahydropyranyl, pyrazolyl, pyrazinyl, thiazolidinyl, indanyl, thienyl, isoxazolyl, pyridazinyl, thiadiazolyl, pyrrolyl, furanyl, thiazolyl, indolyl, imidazolyl, pyrimidinyl, benzimidazolyl, triazolyl, tetrazolylandpyridinyl. Preferred ring systems include cyclopropyl, isoxazolyl and pyrazolyl.

In an embodiment of the invention, R¹⁴ represents a group selected from C₁-C₆ alkyl or C₁-C₄ alkyl, and a saturated or unsaturated 3- to 6-membered ring system optionally comprising one or two ring heteroatoms independently selected from nitrogen, oxygen and sulphur; each group being optionally substituted by one or two substituents independently selected from halogen, cyano, hydroxyl, nitro, —S(O)ₚR³³, —C(O)NR³¹R³², C₁-C₄ alkyl, C₁-C₄ alkoxy, C₂-C₄ alkanoyl, C₁-C₃ alkyl substituted by one or more F atoms, C₁-C₃ alkoxy substituted by one or more F atoms, NR¹²R¹³ and C═CR³⁰.

In an embodiment of the invention, R¹⁴ represents a group selected from C₁-C₄ alkyl and a saturated or unsaturated 3- to 6-membered ring system optionally comprising one or two ring heteroatoms independently selected from nitrogen, oxygen and sulphur; each group being optionally substituted by one or two substituents independently selected from halogen, cyano, nitro, CF₃ and C≡CH.

In a further embodiment of the invention, R¹⁴ represents phenyl or a 5- or 6-membered heteroaromatic ring system comprising one to three ring heteroatoms independently selected from nitrogen, oxygen and sulphur; each ring being optionally substituted by one or two substituents independently selected from F, Cl, Br, cyano, nitro, CF₃ and C≡CH.

Examples of a 5- or 6-membered heteroaromatic ring include furanyl, thienyl, pyrrolyl, oxazolyl, 1,2,4-oxadiazolyl, 1,3,4-oxadiazolyl, isoxazolyl, imidazolyl, pyrazolyl, thiazolyl, triazolyl, tetrazolyl, thiadiazolyl, pyridinyl, pyrimidinyl and pyrazinyl. Preferred heteroaromatic rings include thienyl, imidazolyl, pyridinyl, pyrimidinyl and pyrazinyl, especially pyridinyl.

In a further embodiment of the invention, R¹⁴ represents phenyl optionally substituted by one or two substituents independently selected from F, Cl, Br, cyano, nitro, CF₃ and C≡CH.

In one embodiment, R⁶ represents H.

In one embodiment, R³ represents a phenyl or pyridinyl ring substituted with at least one substituent (e.g. one, two or three substituents) independently selected from halogen (e.g. fluorine, chlorine, bromine or iodine), cyano, nitro, methyl, trifluoromethyl or methylcarbonyl.

In one embodiment, R³ represents a phenyl group substituted with one or two substituents independently selected from fluorine, chlorine, cyano, nitro, trifluoromethyl or methylcarbonyl.

In another embodiment, R³ represents a phenyl group substituted with one or two substituents selected from fluorine, chlorine or trifluoromethyl.

In still another embodiment, R³ represents a phenyl group substituted with a trifluoromethyl substituent (preferably in the meta position).

R⁴ represents hydrogen or C₁-C₆ alkyl (e.g. methyl, ethyl, n-propyl, isopropyl, n-butyl, isobutyl, tert-butyl, n-pentyl or n-hexyl) optionally substituted with at least one substituent (e.g. one or two substituents) independently selected from fluoro, hydroxyl and C₁-C₆ alkoxy (e.g. methoxy, ethoxy, n-propoxy, isopropoxy, n-butoxy, isobutoxy, tert-butoxy, n-pentoxy or n-hexoxy).

In one embodiment, R⁴ represents hydrogen or C₁-C₄ alkyl optionally substituted with one or two substituents independently selected from hydroxyl and C₁-C₄ alkoxy.

In another embodiment, R⁴ represents hydrogen.

X represents a single bond, O, NR²⁴ or a group —C₁-C₆ alkylene-Y—; said allylene being optionally further substituted by OH, halogen, CN, NR³⁷R³⁸, C₁-C₃ alkoxy, CONR³⁹R⁴⁰, SO₂R⁴¹ or SO₂NR⁴²R⁴³. For the avoidance of doubt, X is orientated such that Y is attached to R⁵ in formula (I).

In an embodiment of the invention, Y represents a single bond and the alkylene moiety is a linear or branched C₁-C₆ or C₁-C₄ alkylene, optionally substituted by OH, halogen, CN or C₁-C₃ alkoxy.

In an embodiment of the invention, Y represents a single bond and the alkylene moiety is a linear or branched C₁-C₄ alkylene, optionally substituted by OH, F, CN or OCH₃.

In another embodiment of the invention, X represents methylene.

R⁵ represents a monocyclic ring system selected from
i) phenoxy,
ii) phenyl,
iii) a 5- or 6-membered heteroaromatic ring comprising at least one ring heteroatom (e.g. one, two, three or four ring heteroatoms) independently selected from nitrogen, oxygen and sulphur,
iv) a saturated or partially unsaturated C₃-C₆ hydrocarbyl ring, or
v) a saturated or partially unsaturated 4- to 7-membered heterocyclic ring comprising at least one ring heteroatom (e.g. one, two, three or four ring heteroatoms) independently selected from oxygen, S(O)ᵣ and NR²⁰, wherein at least one of the ring carbon atoms may be optionally replaced by a carbonyl group,

or R⁵ represents a bicyclic ring system in which the two rings are independently selected from the monocyclic ring systems defined in ii), iii), iv) and v) above, wherein the two rings are either fused together, bonded directly to one another or are separated from one another by a linker group selected from oxygen, S(O)ₜ or C₁-C₆ alkylene optionally comprising one or more (e.g. one or two) internal or terminal heteroatoms selected from oxygen, sulphur and NR²⁷ and being optionally substituted by at least one substituent (e.g. one or two substituents) independently selected from hydroxyl, oxo and C₁-C₆ alkoxy (e.g. methoxy, ethoxy, n-propoxy, isopropoxy, n-butoxy, isobutoxy, tert-butoxy, n-pentoxy or n-hexoxy),

the monocyclic or bicyclic ring system being optionally substituted (on a ring atom) by at least one substituent (e.g. one, two or three substituents) independently selected from oxygen (e.g. to form an N-oxide), —S(O)ᵥR²¹, C₁-C₆ alkyl (e.g. methyl, ethyl, n-propyl, isopropyl, n-butyl, isobutyl, tert-butyl, n-pentyl or n-hexyl), CN, OH, C₁-C₆ alkoxy (e.g. methoxy, ethoxy, n-propoxy, isopropoxy, n-butoxy, isobutoxy, tert-butoxy, n-pentoxy or n-hexoxy), halogen (e.g. fluorine, chlorine, bromine or iodine), NR⁴⁷R⁴⁸, NO₂, OSO₂R⁴⁹CO₂R⁵⁰, C(═NH)NH₂, C(O)NR⁵¹R⁵², C(S)NR⁵³R⁵⁴, SC(═NH)NH₂, NR⁵⁵C(═NH)NH₂, SO₂NR⁵⁶R⁵⁷, C₁-C₃ alkyl substituted by SO₂R⁵⁸ or by one or more F atoms (e.g. CH₂SO₂R⁵⁸, CH₂CH₂SO₂R⁵⁸, CH(SO₂R⁵⁸)CH₃, CH₂F, CHF₂, CF₃, CH₂CH₂F, CH₂CF₃, CF₂CF₃, CH(CF₃)₂ and CH₂CH₂CF₃) and C₁-C₃ alkoxy substituted by one or more F atoms (e.g. OCH₂F, OCHF₂, OCF₃, OCH₂CH₂F, OCH₂CF₃, OCF₂CF₃, OCH(CF₃)₂ and OCH₂CH₂CF₃); said C₁-C₆ alkyl being optionally further substituted with at least one substituent selected from cyano, hydroxyl, C₁-C₆ alkoxy (e.g. methoxy, ethoxy, n-propoxy, isopropoxy, n-butoxy, isobutoxy, tert-butoxy, n-pentoxy or n-hexoxy), C₁-C₆ alkylthio (e.g. methylthio, ethylthio, n-propylthio, isopropylthio, n-butylthio, isobutylthio, tert-butylthio, n-pentylthio or n-hexylthio) and —C(O)NR²²R²³.

Or R⁵ may also represent hydrogen.

Examples of a 5- or 6-membered heteroaromatic ring include furanyl, thienyl, pyrrolyl, oxazolyl, 1,2,4-oxadiazolyl, 1,3,4-oxadiazolyl, isoxazolyl, imidazolyl, pyrazolyl, thiazolyl, triazolyl, tetrazolyl, thiadiazolyl, pyridinyl, pyrimidinyl and pyrazinyl. Preferred heteroaromatic rings include isoxazolyl, pyridinyl, imidazolyl and triazolyl.

Unless otherwise indicated, a “saturated or partially unsaturated C₃-C₆ hydrocarbyl ring” denotes a 3- to 6-membered non-aromatic hydrocarbyl ring optionally incorporating one or more double bonds, examples of which include cyclopropyl, cyclobutyl, cyclopentyl, cyclohexyl, cyclopentenyl and cyclohexenyl. A preferred hydrocarbyl ring is cyclopropyl.

Unless otherwise indicated, a “saturated or partially unsaturated 4- to 7-membered heterocyclic ring” as specified above denotes a 4- to 7-membered non-aromatic heterocyclic ring optionally incorporating one or more double bonds and optionally incorporating a carbonyl group, examples of which include tetrahydrofuranyl, tetramethylene sulfonyl, tetrahydropyranyl, 4-oxo-4H-pyranyl (4H-pyran-4-onyl), pyrrolidinyl, 3-pyrrolinyl, imidazolidinyl, 1,3-dioxolanyl (1,3-dioxacyclopentanyl), piperidinyl, piperazinyl, morpholinyl, perhydroazepinyl (hexamethylene iminyl), pyrrolidonyl and piperidonyl. A preferred saturated or partially unsaturated 4- to 7-membered heterocyclic ring is pyrrolidonyl.

Examples of bicyclic ring systems in which the two rings are either fused together, bonded directly to one another or are separated from one another by a linker group include biphenyl, thienylphenyl, pyrazolylphenyl, phenoxyphenyl, phenylcyclopropyl, naphthyl, indanyl, quinolyl, tetrahydroquinolyl, benzofuranyl, indolyl, isoindolyl, indolinyl, benzofuranyl, benzothienyl, indazolyl, benzimidazolyl, benzthiazolyl, purinyl, isoquinolyl, chromanyl, indenyl, quinazolyl, quinoxalyl, chromanyl, isocromanyl, 3H-indolyl, 1H-indazolyl, quinuclidyl, tetrahydronaphthyl, dihydrobenzofuranyl, morpholine-4-ylphenyl, 1,3-benzodioxolyl, 2,3-dihydro-1,4-benzodioxinyl, 1,3-benzodioxinyl and 3,4-dihydro-isochromenyl.

In an embodiment of the invention, R⁵ represents a substituted monocyclic ring system as defined above.

In another embodiment of the invention, R⁵ represents a substituted bicyclic ring system as defined above.

In another embodiment of the invention, R⁵ represents H.

In a further embodiment of the invention, R⁵ represents a monocyclic ring system selected from
i) phenoxy,
ii) phenyl,
iii) a 5- or 6-membered heteroaromatic ring comprising one or two ring heteroatoms independently selected from nitrogen, oxygen and sulphur,
iv) a saturated or partially unsaturated C₃-C₆ hydrocarbyl ring, or
v) a saturated or partially unsaturated 4- to 7-membered heterocyclic ring comprising one or two ring heteroatoms independently selected from oxygen, S(O)ᵣ and NR²⁰, wherein at least one of the ring carbon atoms may be optionally replaced by a carbonyl group,

or R⁵ represents a bicyclic ring system in which the two rings are independently selected from the monocyclic ring systems defined in ii), iii), iv) and v) above, wherein the two rings are either fused together, bonded directly to one another or are separated from one another by a linker group selected from oxygen, methylene and S(O)ₜ,

the monocyclic or bicyclic ring system being substituted by one or two substituents independently selected from OH, —S(O)ᵥR²¹ and C₁-C₄ alkyl.

In a still further embodiment of the invention, R⁵ represents a monocyclic ring system selected from phenyl or a 5- or 6-membered heteroaromatic ring comprising one or two ring heteroatoms independently selected from nitrogen and oxygen, the monocyclic ring system being substituted by one or two substituents independently selected from OH, —S(O)ᵥR²¹ and C₁-C₄ alkyl.

In one embodiment p is 2.

R²⁰ represents hydrogen, C₁-C₆ alkyl (e.g. methyl, ethyl, n-propyl, isopropyl, n-butyl, isobutyl, tert-butyl, n-pentyl or n-hexyl), C₁-C₆ alkylcarbonyl (e.g. methylcarbonyl (acetyl), ethylcarbonyl, n-propylcarbonyl, isopropylcarbonyl, n-butylcarbonyl, isobutylcarbonyl, tert-butylcarbonyl, n-pentylcarbonyl or n-hexylcarbonyl), or C₁-C₆ alkoxycarbonyl (e.g. methoxycarbonyl, ethoxycarbonyl, n-propoxycarbonyl, isopropoxycarbonyl, n-butoxycarbonyl, isobutoxycarbonyl, tert-butoxycarbonyl, n-pentoxycarbonyl or n-hexoxycarbonyl).

In a further embodiment, R²⁰ represents hydrogen, methyl, ethyl, methylcarbonyl (acetyl), ethylcarbonyl, methoxycarbonyl or ethoxycarbonyl.

In one embodiment, v is 2.

R²¹ represents hydrogen, C₁-C₆ alkyl (e.g. methyl, ethyl, n-propyl, isopropyl, n-butyl, isobutyl, tert-butyl, n-pentyl or n-hexyl) or C₃-C₈ cycloalkyl (cyclopropyl, cyclobutyl, cyclopentyl, cyclohexyl, cycloheptyl or cyclooctyl); said allyl or cycloalkyl group being optionally further substituted by one or more substituents selected independently from OH, CN, C₁-C₃ alkoxy and CONR⁵⁹R⁶⁰.

In an embodiment according to the invention, R²¹ represents C₁-C₄ alkyl or C₃-C₆ cycloalkyl.

In another embodiment, R²¹ represents C₁-C₃ alkyl (particularly methyl, ethyl or isopropyl) or cyclopropyl.

R⁴¹ represents hydrogen, C₁-C₆ alkyl (e.g. methyl, ethyl, n-propyl, isopropyl, n-butyl, isobutyl, tert-butyl, n-pentyl or n-hexyl) or C₃-C₈ cycloallyl (cyclopropyl, cyclobutyl, cyclopentyl, cyclohexyl, cycloheptyl or cyclooctyl).

In an embodiment according to the invention, R⁴¹ represents C₁-C₄ alkyl or C₃-C₆ cycloalkyl.

In another embodiment, R⁴¹ represents C₁-C₃ alkyl (particularly methyl, ethyl or isopropyl) or cyclopropyl.

R¹⁰, R¹¹, R¹² and R¹³ each independently represent hydrogen or C₁-C₆ alkyl (e.g. methyl, ethyl, n-propyl, isopropyl, n-butyl, isobutyl, tert-butyl, n-pentyl or n-hexyl).

In an embodiment of the invention, R¹⁰, R¹¹, R¹² and R¹³ each independently represent hydrogen or methyl.

R¹⁵, R¹⁶, R¹⁷, R¹⁸ and R¹⁹ each independently represent hydrogen or C₁-C₆ alkyl (e.g. methyl, ethyl, n-propyl, isopropyl, n-butyl, isobutyl, tert-butyl, n-pentyl or n-hexyl).

In an embodiment of the invention, R¹⁵, R¹⁶, R¹⁷, R¹⁸ and R¹⁹ each independently represent hydrogen or methyl.

R²² and R²³ each independently represent hydrogen or C₁-C₆ alkyl (e.g. methyl, ethyl, n-propyl, isopropyl, n-butyl, isobutyl, tert-butyl, n-pentyl or n-hexyl).

In an embodiment of the invention, R²² and R²³ each independently represent hydrogen.

R²⁴ represents hydrogen or C₁-C₆ alkyl (e.g. methyl, ethyl, n-propyl, isopropyl, n-butyl, isobutyl, tert-butyl, n-pentyl or n-hexyl).

In an embodiment of the invention, R²⁴ represents hydrogen.

R²⁷ represents hydrogen or C₁-C₆ alkyl (e.g. methyl, ethyl, n-propyl, isopropyl, n-butyl, isobutyl, tert-butyl, n-pentyl or n-hexyl).

In an embodiment of the invention, R²⁷ represents hydrogen.

In an embodiment of the invention,
R¹ represents methyl;
W represents S(O);
Z represents a single bond;
R¹⁴ represents phenyl optionally substituted by one or two substituents independently selected from cyano, F, Cl, Br, CF₃, NO₂ and —C═CH;
R⁶ represents H;
R³ represents a phenyl group substituted with a trifluoromethyl substituent;
R⁴ represents hydrogen;
X represents methylene; and
R⁵ represents phenyl or pyridinyl substituted by —S(O)ᵥR²¹ wherein v represents the integer 2.

In an embodiment of the invention,
R¹ represents methyl;
W represents S(O);
Z represents a single bond;
R¹⁴ represents phenyl optionally substituted by one or two substituents independently selected from cyano, F, Cl, Br, CF₃, NO₂ and —C—CH;
R⁶ represents H;
R³ represents a phenyl group substituted with a trifluoromethyl substituent;
R⁴ represents hydrogen;
X represents a linear or branched C₁-C₄ alkylene optionally substituted by OH, F, CN or OCH₃; and
R⁵ represents H.

Examples of compounds of the invention include
N-Cyclopropyl-5-[(4-methoxyphenyl)sulfinyl]-6-methyl-2-oxo-1-[3-(trifluoromethyl)phenyl]-1,2-dihydropyridine-3-carboxamide;
2-Oxo-N-[3-(2-oxopyrrolidin-1-yl)propyl]-5-(phenylsulfinyl)-1-[3-(trifluoromethyl)phenyl]-1,2-dihydropyridine-3-carboxamide;
5-[(4-Bromophenyl)sulfinyl]-6-methyl-N-[4-(methylsulfonyl)benzyl]-2-oxo-1-[3-(trifluoromethyl)phenyl]-1,2-dihydropyridine-3-carboxamide;
5-[(2,4-Dimethoxybenzyl)sulfinyl]-6-methyl-N-[4-(methylsulfonyl)benzyl]-2-oxo-1-[3-(trifluoromethyl)phenyl]-1,2-dihydropyridine-3-carboxamide;
5-[(4-Cyanophenyl)sulfinyl]-N-cyclopropyl-6-methyl-2-oxo-1-[3-(trifluoromethyl)phenyl]-1,2-dihydropyridine-3-carboxamide;
N-{[5-(Cyclopropylsulfonyl)pyridin-2-yl]methyl}-2-oxo-5-(phenylsulfinyl)-1-[3-(trifluoromethyl)phenyl]-1,2-dihydropyridine-3-carboxamide;
6-Methyl-5-(methylsulfinyl)-N-{[5-(methylsulfonyl)pyridin-2-yl]methyl}-2-oxo-1-[3-(trifluoromethyl)phenyl]-1,2-dihydropyridine-3-carboxamide;
N-Cyclopropyl-5-[(3-methoxyphenyl)sulfinyl]-6-methyl-2-oxo-1-[3-(trifluoromethyl)phenyl]-1,2-dihydropyridine-3-carboxamide;
N-Cyclopropyl-5-[(2-methoxyphenyl)sulfinyl]-6-methyl-2-oxo-1-[3-(trifluoromethyl)phenyl]-1,2-dihydropyridine-3-carboxamide;
5-[(4-Cyanophenyl)sulfinyl]-N-[(2S)-2-hydroxypropyl]-6-methyl-2-oxo-1-[3-(trifluoromethyl)phenyl]-1,2-dihydropyridine-3-carboxamide;
5-[(4-Cyanophenyl)sulfinyl]-6-methyl-2-oxo-1-[3-(trifluoromethyl)phenyl]-1,2-dihydropyridine-3-carboxamide;
5-[(4-Cyanophenyl)sulfinyl]-6-methyl-N-[4-(methylsulfonyl)benzyl]-2-oxo-1-[3-(trifluoromethyl)phenyl]-1,2-dihydropyridine-3-carboxamide;
5-[(2-Cyanoethyl)sulfinyl]-6-methyl-N-[4-(methylsulfonyl)benzyl]-2-oxo-1-[3-(trifluoromethyl)phenyl]-1,2-dihydropyridine-3-carboxamide;
5-[(4-Cyanophenyl)sulfinyl]-N-cyclopropyl-1-(3,5-difluorophenyl)-6-methyl-2-oxo-1,2-dihydropyridine-3-carboxamide;
5-[(4-Cyanophenyl)sulfinyl]-N-{[5-(ethylsulfonyl)pyridin-2-yl]methyl}-6-methyl-2-oxo-1-[3-(trifluoromethyl)phenyl]-1,2-dihydropyridine-3-carboxamide;
5-[(4-Cyanophenyl)sulfinyl]-1-(3,5-difluorophenyl)-N-{[5-(ethylsulfonyl)pyridin-2-yl]methyl}-6-methyl-2-oxo-1,2-dihydropyridine-3-carboxamide;
5-[(4-Cyanophenyl)sulfinyl]-1-(3,5-dichlorophenyl)-N-{[5-(ethylsulfonyl)pyridin-2-yl]methyl}-6-methyl-2-oxo-1,2-dihydropyridine-3-carboxamide;
5-[(4-Cyanophenyl)sulfinyl]-N,6-dimethyl-2-oxo-1-[3-(trifluoromethyl)phenyl]-1,2-dihydropyridine-3-carboxamide;
5-[(4-Cyanophenyl)sulfinyl]-1-(3,5-dichlorophenyl)-N,6-dimethyl-2-oxo-1,2-dihydropyridine-3-carboxamide;
5-[(4-Cyanophenyl)sulfinyl]-1-(3,5-difluorophenyl)-N-[2-(1H-imidazol-4-yl)ethyl]-6-methyl-2-oxo-1,2-dihydropyridine-3-carboxamide;
5-[(4-Cyanophenyl)sulfinyl]-1-(3,5-difluorophenyl)-6-methyl-N-(2-morpholin-4-ylethyl)-2-oxo-1,2-dihydropyridine-3-carboxamide;
5-[(4-Cyanophenyl)sulfinyl]-1-(3,5-difluorophenyl)-N,6-dimethyl-2-oxo-1,2-dihydropyridine-3-carboxamide;
5-[(4-Cyanophenyl)sulfinyl]-6-methyl-N-[(3-methylisoxazol-5-yl)methyl]-2-oxo-1-[3-(trifluoromethyl)phenyl]-1,2-dihydropyridine-3-carboxamide;
N-Cyclopropyl-5-[(4-hydroxyphenyl)sulfinyl]-6-methyl-2-oxo-1-[3-(trifluoromethy)phenyl]-1,2-dihydropyridine-3-carboxamide;
5-[(4-Cyanophenyl)sulfinyl]-N-[3-(1H-imidazol-1-yl)propyl]-6-methyl-2-oxo-1-[3-(trifluoromethyl)phenyl]-1,2-dihydropyridine-3-carboxamide;
5-[(4-Cyanophenyl)sulfinyl]-6-methyl-2-oxo-N-[3-(1H-1,2,3-triazol-1-yl)propyl]-1-[3-(trifluoromethyl)phenyl]-1,2-dihydropyridine-3-carboxamide;
5-[(4-Cyanophenyl)sulfinyl]-N-[(1-hydroxycyclopropyl)methyl]-6-methyl-2-oxo-1-[3-(trifluoromethyl)phenyl]-1,2-dihydropyridine-3-carboxamide;
1-(3-Cyanophenyl)-5-[(4-cyanophenyl)sulfinyl]-6-methyl-N-{[5-(methylsulfonyl)pyridin-2-yl]methyl}-2-oxo-1,2-dihydropyridine-3-carboxamide;
5-[(4-Cyanophenyl)sulfinyl]-N-(2-methoxyethyl)-6-methyl-2-oxo-1-[3-(trifluoromethyl)phenyl]-1,2-dihydropyridine-3-carboxamide;
5-[(4-Cyanophenyl)sulfinyl]-N-(2-hydroxy-2-methylpropyl)-6-methyl-2-oxo-1-[3-(trifluoromethyl)phenyl]-1,2-dihydropyridine-3-carboxamide;
5-[(4-Chlorophenyl)sulfinyl]-6-methyl-N-[4-(methylsulfonyl)benzyl]-2-oxo-1-[3-(trifluoromethyl)phenyl]-1,2-dihydropyridine-3-carboxamide;
6-Methyl-5-[(4-methylphenyl)sulfinyl]-N-[4-(methylsulfonyl)benzyl]-2-oxo-1-[3-(trifluoromethyl)phenyl]-1,2-dihydropyridine-3-carboxamide;
6-Methyl-N-[4-(methylsulfonyl)benzyl]-5-[(4-nitrophenyl)sulfinyl]-2-oxo-1-[3-(trifluoromethyl)phenyl]-1,2-dihydropyridine-3-carboxamide;
6-Methyl-N-[4-(methylsulfonyl)benzyl]-2-oxo-1-[3-(trifluoromethyl)phenyl]-5-{[4-(trifluoromethyl)phenyl]sulfinyl}-1,2-dihydropyridine-3-carboxamide;
5-{[4-(Acetylamino)phenyl]sulfinyl}-6-methyl-N-[4-(methylsulfonyl)benzyl]-2-oxo-1-[3-(trifluoromethyl)phenyl]-1,2-dihydropyridine-3-carboxamide;
5-[(4-Ethylphenyl)sulfinyl]-6-methyl-N-[4-(methylsulfonyl)benzyl]-2-oxo-1-[3-(trifluoromethyl)phenyl]-1,2-dihydropyridine-3-carboxamide;
5-[(4-Fluorophenyl)sulfinyl]-6-methyl-N-[4-(methylsulfo-3-yl)benzyl]-2-oxo-1-[3-(trifluoromethyl)phenyl]-1,2-dihydropyridine-3-carboxamide;
5-[(4-Cyanophenyl)sulfinyl]-6-methyl-1-(3-methylphenyl)-N-{[5-(methylsulfonyl)pyridin-2-yl]methyl}-2-oxo-1,2-dihydropyridine-3-carboxamide;
5-[(4-Cyanophenyl)sulfinyl]-N-ethyl-6-methyl-2-oxo-1-[3-(trifluoromethyl)phenyl]-1,2-dihydropyridine-3-carboxamide;
5-[(4-Chlorophenyl)sulfinyl]-N,6-dimethyl-2-oxo-1-[3-(trifluoromethyl)phenyl]-1,2-dihydropyridine-3-carboxamide;
N-Ethyl-5-[(4-fluorophenyl)sulfinyl]-6-methyl-2-oxo-1-[3-(trifluoromethyl)phenyl]-1,2-dihydropyridine-3-carboxamide;
5-[(4-Fluorophenyl)sulfinyl]-N,6-dimethyl-2-oxo-1-[3-(trifluoromethyl)phenyl]-1,2-dihydropyridine-3-carboxamide;
5-[(4-Bromophenyl)sulfinyl]-N,6-dimethyl-2-oxo-1-[3-(trifluoromethyl)phenyl]-1,2-dihydropyridine-3-carboxamide;
5-[(4-Cyanophenyl)sulfinyl]-N-(2-hydroxyethyl)-6-methyl-2-oxo-1-[3-(trifluoromethyl)phenyl]-1,2-dihydropyridine-3-carboxamide;
5-[(4-Cyanophenyl)sulfinyl]-N-(cyclopropylmethyl)-6-methyl-2-oxo-1-[3-(trifluoromethyl)phenyl]-1,2-dihydropyridine-3-carboxamide;
N-Methyl-2-oxo-5-(phenylsulfinyl)-1-[3-(trifluoromethyl)phenyl]-1,2-dihydropyridine-3-carboxamide;

N—(Cyanomethyl)-5-[(4-cyanophenyl)sulfinyl]-6-methyl-2-oxo-1-[3-(trifluoromethyl)phenyl]-1,2-dihydropyridine-3-carboxamide;
5-[(4-Cyanophenyl)sulfinyl]-N-[2-(1H-imidazol-4-yl)ethyl]-6-methyl-2-oxo-1-[3-(trifluoromethyl)phenyl]-1,2-dihydropyridine-3-carboxamide;
5-[(4-Cyanophenyl)sulfinyl]-N-(2-hydroxypropyl)-6-methyl-2-oxo-1-[3-(trifluoromethyl)phenyl]-1,2-dihydropyridine-3-carboxamide;
5-[(4-Cyanophenyl)sulfinyl]-6-methyl-N-(2-morpholin-4-ylethyl)-2-oxo-1-[3-(trifluoromethyl)phenyl]-1,2-diliydropyridine-3-carboxamide;
5-[(4-Cyanophenyl)sulfinyl]-N-(2-hydroxy-1,1-dimethylethyl)-6-methyl-2-oxo-1-[3-(trifluoromethyl)phenyl]-1,2-dihydropyridine-3-carboxamide;
5-[(4-Cyanophenyl)sulfinyl]-N,6-dimethyl-2-oxo-1-[3-(trifluoromethyl)phenyl]-1,2-dihydropyridine-3-carboxamide;
5-[(4-Cyanophenyl)sulfinyl]-N-[(2R)-2-hydroxypropyl]-6-methyl-2-oxo-1-[3-(trifluoromethyl)phenyl]-1,2-dihydropyridine-3-carboxamide;
5-[(4-Cyanophenyl)sulfinyl]-6-methyl-2-oxo-N-[3-(2-oxopyrrolidin-1-yl)propyl]-1-[3-(trifluoromethyl)phenyl]-1,2-dihydropyridine-3-carboxamide;
5-[(4-Cyanophenyl)sulfinyl]-N-(2-methoxypropyl)-6-methyl-2-oxo-1-[3-(trifluoromethyl)phenyl]-1,2-dihydropyridine-3-carboxamide;
6-Methyl-5-(methylsulfonyl)-N-{[5-(methylsulfonyl)pyridin-2-yl]methyl}-2-oxo-1-[3-(trifluoromethyl)phenyl]-1,2-dihydropyridine-3-carboxamide;
2-Oxo-N-[3-(2-oxopyrrolidin-1-yl)propyl]-5-(phenylsulfonyl)-1-[3-(trifluoromethyl)phenyl]-1,2-dihydropyridine-3-carboxamide;
5-[(4-Cyanophenyl)sulfonyl]-N,6-dimethyl-2-oxo-1-[3-(trifluoromethyl)phenyl]-1,2-dihydropyridine-3-carboxamide;
5-{[4-(Acetylamino)phenyl]sulfonyl}-6-methyl-N-[4-(methylsulfonyl)benzyl]-2-oxo-1-[3-(trifluoromethyl)phenyl]-1,2-dihydropyridine-3-carboxamide;
5-[(4-Ethylphenyl)sulfonyl]-6-methyl-N-[4-(methylsulfonyl)benzyl]-2-oxo-1-[3-(trifluoromethyl)phenyl]-1,2-dihydropyridine-3-carboxamide;
5-[(4-Cyanophenyl)sulfonyl]-N,6-dimethyl-2-oxo-1-[3-(trifluoromethyl)phenyl]-1,2-dihydropyridine-3-carboxamide;
5-[(4-Cyanophenyl)sulfonyl]-N-(2-hydroxy-1,1-dimethylethyl)-6-methyl-2-oxo-1-[3-(trifluoromethyl)phenyl]-1,2-dihydropyridine-3-carboxamide;
N-[(3-Cyclopropylisoxazol-5-yl)methyl]-6-methyl-5-(methylsulfonyl)-2-oxo-1-[3-(trifluoromethyl)phenyl]-1,2-dihydropyridine-3-carboxamide;
5-[(6-Cyanopyridin-3-yl)sulfonyl]-N,6-dimethyl-2-oxo-1-[3-(trifluoromethyl)phenyl]-1,2-dihydropyridine-3-carboxamide;
6-Methyl-N-[4-(methylsulfonyl)benzyl]-2-oxo-1-[3-(trifluoromethyl)phenyl]-5-({4-[(trimethylsilyl)ethynyl]phenyl}sulfinyl)-1,2-dihydropyridine-3-carboxamide;
5-[(4-Ethynylphenyl)sulfinyl]-6-methyl-N-[4-(methylsulfonyl)benzyl]-2-oxo-1-[3-(trifluoromethyl)phenyl]-1,2-dihydropyridine-3-carboxamide;
6-Methyl-N-[4-(methylsulfonyl)benzyl]-2-oxo-5-{[4-(phenylethynyl)phenyl]sulfinyl}-1-[3-(trifluoromethyl)phenyl]-1,2-dihydropyridine-3-carboxamide;
6-Methyl-N-[4-(methylsulfonyl)benzyl]-2-oxo-5-[(4-prop-1-yn-1-ylphenyl)sulfinyl]-1-[3-(trifluoromethyl)phenyl]-1,2-dihydropyridine-3-carboxamide;
5-[(5-Cyanopyridin-2-yl)sulfinyl]-N,6-dimethyl-2-oxo-1-[3-(trifluoromethyl)phenyl]-1,2-dihydropyridine-3-carboxamide;
6-({2-Methyl-5-(methylcarbamoyl)-6-oxo-1-[3-(trifluoromethyl)phenyl]-1,6-dihydropyridin-3-yl}sulfinyl)nicotinamide;
5-[(5-Chloropyridin-2-yl)sulfinyl]-N,6-dimethyl-2-oxo-1-[3-(trifluoromethyl)phenyl]-1,2-dihydropyridine-3-carboxamide;
5-[(5-Bromopyridin-2-yl)sulfinyl]-N,6-dimethyl-2-oxo-1-[3-(trifluoromethyl)phenyl]-1,2-dihydropyridine-3-carboxamide;
5-[(5-Cyanopyridin-2-yl)sulfinyl-3-yl]-6-methyl-N-[4-(methylsulfonyl)benzyl]-2-oxo-1-[3-(trifluoromethyl)phenyl]-1,2-dihydropyridine-3-carboxamide;
5-[(5-Bromopyrimidin-2-yl)sulfinyl]-N,6-dimethyl-2-oxo-1-[3-(trifluoromethyl)phenyl]-1,2-dihydropyridine-3-carboxamide;
5-[(6-Bromopyridazin-3-yl)sulfinyl]-N,6-dimethyl-2-oxo-1-[3-(trifluoromethyl)phenyl]-1,2-dihydropyridine-3-carboxamide;
5-[(6-Cyanopyridin-3-yl)sulfinyl]-N,6-dimethyl-2-oxo-1-[3-(trifluoromethyl)phenyl]-1,2-dihydropyridine-3-carboxamide;
5-[(5-Cyano-2-thienyl)sulfinyl]-N,6-dimethyl-2-oxo-1-[3-(trifluoromethyl)phenyl]-1,2-dihydropyridine-3-carboxamide;
5-(1H-Imidazol-2-ylsulfinyl)-6-methyl-N-[4-(methylsulfonyl)benzyl]-2-oxo-1-[3-(trifluoromethyl)phenyl]-1,2-dihydropyridine-3-carboxamide;
6-Methyl-5-[(methylamino)sulfonyl]-N-[4-(methylsulfonyl)benzyl]-2-oxo-1-[3-(trifluoromethyl)phenyl]-1,2-dihydropyridine-3-carboxamide;
5-(Anilinosulfonyl)-6-methyl-N-[4-(methylsulfonyl)benzyl]-2-oxo-1-[3-(trifluoromethyl)phenyl]-1,2-dihydropyridine-3-carboxamide;
6-Methyl-N-[4-(methylsulfonyl)benzyl]-5-{[(2-morpholin-4-ylethyl)amino]sulfonyl}-2-oxo-1-[3-(trifluoromethyl)phenyl]-1,2-dihydropyridine-3-carboxamide;
5-{[(2-Cyanoethyl)(methyl)amino]sulfonyl}-6-methyl-N-[4-(methylsulfonyl)benzyl]-2-oxo-1-[3-(trifluoromethyl)phenyl]-1,2-dihydropyridine-3-carboxamide;
6-Methyl-N-[4-(methylsulfonyl)benzyl]-5-{[(6-morpholin-4-ylpyridin-3-yl)amino]sulfonyl}-2-oxo-1-[3-(trifluoromethyl)phenyl]-1,2-dihydropyridine-3-carboxamide;
6-Methyl-N-[4-(methylsulfonyl)benzyl]-5-(morpholin-4-ylsulfonyl)-2-oxo-1-[3-(trifluoromethyl)phenyl]-1,2-dihydropyridine-3-carboxamide;
6-Methyl-N-[4-(methylsulfonyl)benzyl]-2-oxo-5-[(pyridin-3-ylamino)sulfonyl]-1-[3-(trifluoromethyl)phenyl]-1,2-dihydropyridine-3-carboxamide;
2-Methyl-5-({[4-(methylsulfonyl)benzyl]amino}carbonyl)-6-oxo-1-[3-(trifluoromethyl)phenyl]-1,6-dihydropyridine-3-sulfonic acid;
6-Methyl-N-[4-(methylsulfonyl)benzyl]-2-oxo-5-(phenylthio)-1-[3-(trifluoromethyl)phenyl]-1,2-dihydropyridine-3-carboxamide;
6-Methyl-N-[4-(methylsulfonyl)benzyl]-2-oxo-5-(phenylsulfinyl)-1-[3-(trifluoromethyl)phenyl]-1,2-diliydropyridine-3-carboxamide;
6-Methyl-N-[4-(methylsulfonyl)benzyl]-2-oxo-5-(phenylsulfonyl)-1-[3-(trifluoromethyl)phenyl]-1,2-dihydropyridine-3-carboxamide;
6-Methyl-5-(methylsulfinyl)-N-[4-(methylsulfonyl)benzyl]-2-oxo-1-[3-(trifluoromethyl)phenyl]-1,2-dihydropyridine-3-carboxamide;
6-Methyl-5-(methylsulfonyl)-N-[4-(methylsulfonyl)benzyl]-2-oxo-1-[3-(trifluoromethyl)phenyl]-1,2-dihydropyridine-3-carboxamide;
5-(Benzylsulfinyl)-6-methyl-N-[4-(methylsulfonyl)benzyl]-2-oxo-1-[3-(trifluoromethyl)phenyl]-1,2-dihydropyridine-3-carboxamide;
5-(Ethylsulfinyl)-6-methyl-N-[4-(methylsulfonyl)benzyl]-2-oxo-1-[3-(trifluoromethyl)phenyl]-1,2-dihydropyridine-3-carboxamide;
Methyl 3-({2-methyl-5-({[4-(methylsulfonyl)benzyl]amino}carbonyl)-6-oxo-1-[3-(trifluoromethyl)phenyl]-1,6-dihydropyridin-3-yl}sulfinyl)propanoate;
5-(Cyclohexylsulfinyl)-6-methyl-N-[4-(methylsulfonyl)benzyl]-2-oxo-1-[3-(trifluoromethyl)phenyl]-1,2-dihydropyridine-3-carboxamide;
5-(Cyclopropylsulfonyl)-N-[4-(cyclopropylsulfonyl)benzyl]-6-methyl-2-oxo-1-[3-(trifluoromethyl)phenyl]-1,2-dihydropyridine-3-carboxamide;
   and pharmaceutically acceptable salts of any one thereof.

The present invention further provides a process for the preparation of a compound of formula (I) or a pharmaceutically acceptable salt thereof as defined above which comprises,

(a) reacting a compound of formula (II)

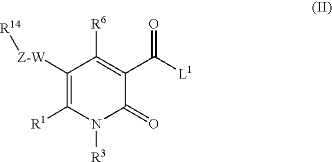

wherein L¹ represents a leaving group (such as halogen or hydroxyl) and R¹, R³, R⁶, R¹⁴, W and Z are as defined in formula (I),
with a compound of formula

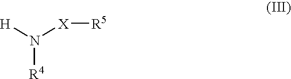

wherein X, R⁴ and R⁵ are as defined in formula (I); or
(b) when W represents —S— and Z represents a single bond or —CH₂—, reacting a compound of formula (IV)

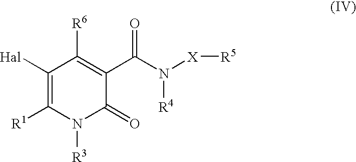

wherein Hal represents a halogen atom and X, R¹, R³, R⁴, R⁵ and R⁶ are as defined in formula (I),
with a nucleophile R¹⁴-Z-S-M wherein R¹⁴ and Z are as defined in formula (I) and M represents an organo-tin or organo boronic acid group; or
(c) when W represents —S— and Z represents a single bond or —CH₂—, reacting a compound of formula (IV) wherein Hal represents a halogen atom and X, R¹, R³, R⁴, R⁵ and R⁶ are as defined in formula (I),
with a thiol R¹⁴-Z-S—H wherein R¹⁴ and Z are as defined in formula (I) in the presence of a copper (I) salt; or
(d) when W represents —S— and Z represents a single bond or —CH₂—, reacting a compound of formula (V)

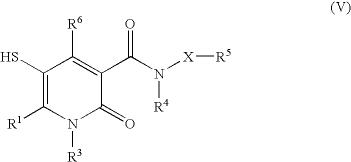

wherein X, R¹, R³, R⁴, R⁵ and R⁶ are as defined in formula (I),
with an electrophile R¹⁴-Z-L wherein L² represents a leaving group such as halogen and R¹⁴ and Z are as defined in formula (I); or
(e) when W represents —SO₂— and Z represents —NR²⁵—, reacting a compound of formula (VI)

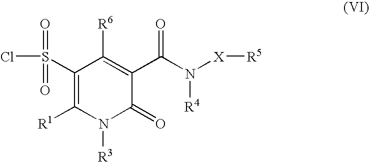

wherein X, R¹, R³, R⁴, R⁵ and R⁶ are as defined in formula (I),
with an amine R¹⁴—NHR²⁵ wherein R¹⁴ and R²⁵ are as defined in formula (I); or
(f) when W represents a sulfinyl (—S(O)—) or a sulfonyl (—S(O)₂—) group, oxidising the corresponding compound wherein W represents a thio (—S—) group;
and optionally after (a), (b), (c), (d), (e) or (f) carrying out one or more of the following:
converting the compound obtained to a further compound of the invention
forming a pharmaceutically acceptable salt of the compound.

In process (a), the reaction may conveniently be carried out in an organic solvent such as dichloromethane or N-methylpyrrolidinone at a temperature, for example, in the range from 0° C. to the boiling point of the solvent. If necessary or desired, a base and/or a coupling reagent such as HATU (O-(7-Azabenzotriazol-1-yl)-N,N,N′,N′-tetramethyluronium hexafluorophosphate), HOAT (1-Hydroxy-7-azabenzotriazole), HOBT (1-Hydroxybenzotriazole hydrate) or DIEA (N,N-Diisopropylethylamine) may be added.

In process (b), the reaction may conveniently be carried out in an organic solvent such as DMF, NMP or toluene or a mixture thereof at elevated temperature (i.e. above ambient temperature, 20° C.), for example, in the range from 50° C. to 150° C. and in the presence of a suitable transition metal catalyst such as bis(tri-t-butylphosphine)palladium. If necessary or desired, a base such as potassium carbonate may be added.

In process (c), the reaction may conveniently be carried out in an organic solvent such as acetonitrile at elevated temperature (i.e. above ambient temperature, 20° C.), for example, in the range from 50° C. to the boiling point, and in the presence of a salt such as copper (I) iodide and an amine such as (±)-trans-cyclohex-1,2-diamine.

In process (d), the reaction may conveniently be carried out in an organic solvent such as acetonitrile or dioxane at elevated temperature (i.e. above ambient temperature, 20° C.), for example, in the range from 40° C. to the boiling point, and in the presence of a salt such as copper (I) iodide and an amine such as (±)-trans-cyclohex-1,2-diamine. Alternatively, the reaction may be carried out in the presence of a base such as caesium carbonate.

In process (e), the reaction may conveniently be carried out in an organic solvent such as tetrahydrofuran, optionally in the presence of a base.

In process (f), the oxidation may conveniently be carried out using hydrogen peroxide or sodium periodate. Other suitable oxidants will be readily apparent to the man skilled in the art.

Specific processes for the preparation of compounds of Formula (I) are disclosed within the Examples section of the present specification. Such processes form an aspect of the present invention.

The necessary starting materials are either commercially available, are known in the literature or may be prepared using known techniques. Specific processes for the preparation of certain key starting materials are disclosed within the Examples section of the present specification and such processes form an aspect of the present invention.

Compounds of formula (I) can be converted into further compounds of formula (I) using standard procedures.

It will be appreciated by those skilled in the art that in the processes of the present invention certain functional groups such as hydroxyl or amino groups may need to be protected by protecting groups. Thus, the preparation of the compounds of formula (I) may involve, at an appropriate stage, the addition and/or removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 3^{rd} edition, T. W. Greene and P. G. M. Wuts, Wiley-Interscience (1999).

The compounds of formula (I) above may be converted to a pharmaceutically acceptable salt thereof, preferably an acid addition salt such as a hydrochloride, hydrobromide, sulphate, phosphate, acetate, fumarate, maleate, tartrate, lactate, citrate, pyruvate, succinate, oxalate, methanesulphonate or p-toluenesulphonate.

Compounds of formula (I) are capable of existing in stereoisomeric forms. It will be understood that the invention encompasses the use of all geometric and optical isomers (including atropisomers) of the compounds of formula (I) and mixtures thereof including racemates. The use of tautomers and mixtures thereof also form an aspect of the present invention. Enantiomerically pure forms are particularly desired.

The compounds of formula (I) and their pharmaceutically acceptable salts have activity as pharmaceuticals, in particular as modulators of serine proteases such as proteinase 3 and pancreatic elastase and, especially, human neutrophil elastase, and may therefore be beneficial in the treatment or prophylaxis of inflammatory diseases and conditions.

Examples of such conditions include: adult respiratory distress syndrome (ARDS), cystic fibrosis, pulmonary emphysema, bronchitis, bronchiectasis, chronic obstructive pulmonary disease (COPD) and ischaemic-reperfusion injury. The compounds of this invention may also be useful in the modulation of endogenous and/or exogenous biological irritants which cause and/or propagate atherosclerosis, diabetes, myocardial infarction; hepatic disorders including but not limited to cirrhosis, systemic lupus erythematous, inflammatory disease of lymphoid origin, including but not limited to T lymphocytes, B lymphocytes, thymocytes; autoimmune diseases, bone marrow; inflammation of the joint (especially rheumatoid arthritis, osteoarthritis and gout); inflammation of the gastro-intestinal tract (especially inflammatory bowel disease, ulcerative colitis, pancreatitis and gastritis); inflammation of the skin (especially psoriasis, eczema, dermatitis); in tumour metastasis or invasion; in disease associated with uncontrolled degradation of the extracellular matrix such as osteoarthritis; in bone resorptive disease (such as osteoporosis and Paget's disease); diseases associated with aberrant angiogenesis; the enhanced collagen remodelling associated with diabetes, periodontal disease (such as gingivitis), corneal ulceration, ulceration of the skin, post-operative conditions (such as colonic anastomosis) and dermal wound healing; demyelinating diseases of the central and peripheral nervous systems (such as multiple sclerosis); age related illness such as dementia, inflammatory diseases of cardiovascular origins; granulomatous diseases; renal diseases including but not limited to nephritis and polyarteritis; cancer; pulmonary hypertension, ingested poisons, skin contacts, stings, bites; asthma; rhinitis; HIV disease progression; for minimising the effects of organ rejection in organ transplantation including but not limited to human organs; and replacement therapy of proteinase inhibitors.

Thus, the present invention provides a compound of formula (I) or a pharmaceutically-acceptable salt thereof as hereinbefore defined for use in therapy.

In a further aspect, the present invention provides the use of a compound of formula (I) or a pharmaceutically acceptable salt thereof as hereinbefore defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

The invention also provides a method of treating, or reducing the risk of, a disease or condition in which inhibition of neutrophil elastase activity is beneficial which comprises administering to a patient in need thereof a therapeutically effective amount of a compound of formula (I) or a pharmaceutically acceptable salt thereof as hereinbefore defined.

The invention still further provides a method of treating, or reducing the risk of, an inflammatory disease or condition which comprises administering to a patient in need thereof a therapeutically effective amount of a compound of formula (I) or a pharmaceutically acceptable salt thereof as hereinbefore defined.

In particular, the compounds of this invention may be used in the treatment of adult respiratory distress syndrome (ARDS), cystic fibrosis, pulmonary emphysema, bronchitis, bronchiectasis, chronic obstructive pulmonary disease (COPD), pulmonary hypertension, asthma, rhinitis, ischemia-reperfusion injury, rheumatoid arthritis, osteoarthritis, cancer, atherosclerosis and gastric mucosal injury.

For the above-mentioned therapeutic uses the dosage administered will, of course, vary with the compound employed, the mode of administration, the treatment desired and the disorder indicated. The daily dosage of the compound of the invention may be in the range from 0.05 mg/kg to 100 mg/kg.

The compounds of formula (I) and pharmaceutically acceptable salts thereof may be used on their own but will generally be administered in the form of a pharmaceutical composition in which the formula (I) compound/salt (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I) or a pharmaceutically acceptable salt thereof as hereinbefore defined, in association with a pharmaceutically acceptable adjuvant, diluent or carrier.

The invention further provides a process for the preparation of a pharmaceutical composition of the invention which comprises mixing a compound of formula (I) or a pharmaceutically acceptable salt thereof as hereinbefore defined with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane (HFA) aerosols and dry powder formulations, for example, formulations in the inhaler device known as the Turbuhaler®; or systemically, e.g. by oral administration in the form of tablets, capsules, syrups, powders or granules; or by parenteral administration in the form of solutions or suspensions; or by subcutaneous administration; or by rectal administration in the form of suppositories; or transdermally.

Dry powder formulations and pressurized HFA aerosols of the compounds of the invention may be administered by oral or nasal inhalation. For inhalation, the compound is desirably finely divided. The finely divided compound preferably has a mass median diameter of less than 10 μm, and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

The compounds of the invention may also be administered by means of a dry powder inhaler. The inhaler may be a single or a multi dose inhaler, and may be a breath actuated dry powder inhaler.

One possibility is to mix the finely divided compound of the invention with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

For oral administration the compound of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compound of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The compounds of the invention may also be administered in conjunction with other compounds used for the treatment of the above conditions.

The present invention will now be further explained by reference to the following illustrative examples.

### General Methods

¹H NMR and ¹³C NMR spectra were recorded on a Varian Inova 400 MHz or a Varian Mercury-VX 300 MHz instrument. The central peaks of chloroform-d (δ_{H} 7.27 ppm), dimethylsulfoxide-d₆ (δ_{H} 2.50 ppm), acetonitrile-d₃ (δ_{H} 1.95 ppm) or methanol-d₄ (δ_{H} 3.31 ppm) were used as internal references. Column chromatography was carried out using silica gel (0.040-0.063 mm, Merck). Unless stated otherwise, starting materials were commercially available. All solvents and commercial reagents were of laboratory grade and were used as received.

The following method was used for LC/MS analysis:

Instrument Agilent 1100; Column Waters Symmetry 2.1×30 mm; Mass APCI; Flow rate 0.7 ml/min; Wavelength 254 nm; Solvent A: water+0.1% TFA; Solvent B: acetonitrile+0.1% TFA; Gradient 15-95%/B 8 min, 95% B1 min.

Analytical chromatography was run on a Symmetry C₁₈-column, 2.1×30 mm with 3.5 μm particle size, with acetonitrile/water/0.1% trifluoroacetic acid as mobile phase in a gradient from 5% to 95% acetonitrile over 8 minutes at a flow of 0.7 ml/min.

The abbreviations or terms used in the examples have the following meanings:
HATU: O-(7-Azabenzotriazol-1-yl)-N,N,N′,N′-tetramethyluronium hexafluorophosphate
HOAT: 1-Hydroxy-7-azabenzotriazole
NMP: 1-N-Methyl-2-pyrrolidinone
THF: Tetrahydrofuran
TFA: Trifluoroacetic acid
DMF: N,N-Dimethylformamide
DCM: Dichloromethane
DIPEA: N,N-Diisopropylethylamine
is EtOAc: Ethyl acetate
MeOH: Methanol
MeCN Acetonitrile
EtOH: Ethanol
NaS₂O₄: Sodium hydrosulphite
DMSO: Dimethyl sulphoxide
SM: Starting material
Ex: Example
Aq: Aqueous
HOAc: Acetic acid
RT: Room temperature
DABCO: 1,4-Diazabicyclo[2.2.2]octane.

### EXAMPLE 1

### N-Cyclopropyl-5-[(4-methoxyphenyl)sulfinyl]-6-methyl-2-oxo-1-[3-(trifluoromethyl)phenyl]-1,2-dihydropyridine-3-carboxamide

A mixture of N-cyclopropyl-5-iodo-6-methyl-2-oxo-1-[3-(trifluoromethyl)phenyl]-1,2-dihydropyridine-3-carboxamide (SM3, 25 mg, 0.054 mmol), tributyl-[(4-methoxyphenyl)thio]stannane (28 mg, 0.065 mmol), palladium-tri-tert-butylphosphine (1:2) (2.6 mg, 0.005 mmol) and NMP (1 ml) under argon was heated in a microwave to 150° C. for 10 min. After filtration, the crude compound was purified on an Xterra C8 column using a gradient of acetonitrile/water. The residue obtained on evaporation was dissolved in acetic acid (3 ml) and hydrogen peroxide (35% in water, 100 μl) was added. The mixture was stirred for 3 h at room temperature. Then diluted with water (5 ml) and extracted with ethyl acetate (3×5 ml). The organic phase was dried (MgSO₄), filtered and evaporated. The residue was purified on an Xterra C8 column using a gradient of acetonitrile/water. Freeze drying of the mixture afforded the title compound (10 mg, 38%).

¹H NMR (300 MHz, CDCl₃) δ 9.09 (s, 1H), 8.80 (d, J=5.5 Hz, 1H), 7.82 (t, J=7.8 Hz, 1H), 7.74 (t, J=8.0 Hz, 1H), 7.61 (d, J=2.9 Hz, 2H), 7.58 (d, J=2.8 Hz, 2H), 7.49-7.36 (m, 2H), 7.07 (d, J=2.9 Hz, 2H), 7.04 (d, J=3.1 Hz, 2H), 3.88 (s, 3H), 2.90 (m, 1H), 2.35 (d, J=5.1 Hz, 3H), 0.77 (dd, J=7.2, 1.0 Hz, 2H), 0.52 (dd, J=6.0, 1.2 Hz, 2H);

APCI-MS m/z: 491.2 [MH]

### EXAMPLES 2 TO 13

The following compounds were synthesised using an analogous method to that described for Example 1.

| Ex. | Compound | ¹H NMR | m/z | SM |
|---|---|---|---|---|
| 2 | 2-Oxo-N-[3-(2-oxopyrrolidin- | 9.26 (t, J = 5.9 Hz, 1H), 8.78 (d, J = 2.8 Hz, | 532.1 | SM2 |
| | 1-yl)propyl]-5- | 1H), 8.27 (d, J = 2.8 Hz, | | |
| | (phenylsulfinyl)-1-[3- | 1H), 8.11 (s, 1H), 7.93 (d, J = 7.5 Hz, | | |
| | (trifluoromethyl)phenyl]-1,2- | 2H), 7.87-7.72 (m, 3H), | | |
| | dihydropyridine-3- | 7.67-7.53 (m, 3H), 3.33-3.09 (m, 6H), | | |
| | carboxamide | 2.16 (t, J = 8.1 Hz, 2H), | | |
| | | 1.86 (quintet, J = 7.6 Hz, 2H), | | |
| | | 1.62 (quintet, J = 6.9 Hz, 2H) | | |
| 3 | 5-[(4-Bromophenyl)sulfinyl]- | 9.65 (t, J = 6.0 Hz, 1H), 8.38 (s, 1H), | 667.0, 669.0 | SM1 |
| | 6-methyl-N-[4- | 8.01 (d, J = 7.9 Hz, 1H), 7.93 (d, J = 7.3 Hz, | | |
| | (methylsulfonyl)benzyl]-2- | 1H), 7.88-7.77 (m, 6H), | | |
| | oxo-1-[3-(trifluoromethyl)- | 7.66 (d, J = 8.5 Hz, 2H), 7.50 (d, J = 8.2 Hz, | | |
| | phenyl]-1,2-dihydropyridine- | 2H), 4.60-4.46 (m, 2H), | | |
| | 3-carboxamide | 3.16 (s, 3H), 2.32 (s, 3H) | | |
| 4 | 5-[(2,4- | 9.74 (t, J = 5.9 Hz, 1H), 8.73 (d, J = 16.0 Hz, | 663.0 | SM1 |
| | Dimethoxybenzyl)sulfinyl]-6- | 1H), 7.95-7.78 (m, 4H), | | |
| | methyl-N-[4- | 7.75 (d, J = 8.2 Hz, 1H), 7.56 (dd, J = 8.2, | | |
| | (methylsulfonyl)benzyl]-2- | 2.1 Hz, 2H), 7.49 (d, J = 7.6 Hz, | | |
| | oxo-1-[3-(trifluoromethyl)- | 1H), 7.03 (d, J = 9.1 Hz, 1H), | | |
| | phenyl]-1,2-dihydropyridine- | 6.59-6.48 (m, 2H), 4.61 (m, 2H), | | |
| | 3-carboxamide | 4.25 (dd, J = 11.9, 9.8 Hz, 1H), | | |
| | | 4.00 (dd, J = 12.0, 3.1 Hz, 1H), | | |
| | | 3.77-3.68 (m, 6H), 3.18 (s, 3H), 1.54 (d, J = 6.2 Hz, | | |
| | | 3H) | | |
| 5 | 5-[(4-Cyanophenyl)sulfinyl]- | 9.05 (s, 1H), 8.31 (d, J = 2.0 Hz, | 486.1 | SM3 |
| | N-cyclopropyl-6-methyl-2- | 1H), 8.10 (d, J = 7.2 Hz, 2H), | | |
| | oxo-1-[3-(trifluoromethyl)- | 8.01-7.74 (m, 6H), 2.76 (m, 1H), 2.34 (s, | | |
| | phenyl]-1,2-dihydropyridine- | 3H), 0.67 (d, J = 7.5 Hz, 2H), | | |
| | 3-carboxamide | 0.45 (q, J = 3.7 Hz, 2H). | | |
| 6 | N-{[5-(Cyclopropylsulfonyl)- | 9.93 (t, J = 5.7 Hz, 1H), 8.94 (dd, J = 2.4, | 602.1 | SM4 |
| | pyridin-2-yl]methyl}-2-oxo- | 0.7 Hz, 1H), 8.83 (d, J = 2.8 Hz, | | |
| | 5-(phenylsulfinyl)-1-[3- | 1H), 8.29 (d, J = 2.8 Hz, 1H), | | |
| | (trifluoromethyl)phenyl]-1,2- | 8.22 (dd, J = 8.3, 2.4 Hz, 1H), | | |
| | dihydropyridine-3- | 8.13 (s, 1H), 8.00-7.51 (m, 9H), 4.69 (d, | | |
| | carboxamide | J = 5.9 Hz, 2H), 2.94 (m, 1H), | | |
| | | 1.19-1.00 (m, 4H) | | |
| 7 | 6-Methyl-5-(methylsulfinyl)- | 9.96 (t, J = 5.7 Hz, 1H), 8.99 (d, J = 2.2 Hz, | 528.1 | SM5 |
| | N-{[5-(methylsulfonyl)- | 1H), 8.79 (d, J = 1.9 Hz, | | |
| | pyridin-2-yl]methyl}-2-oxo- | 1H), 8.27 (dd, J = 8.2, 2.4 Hz, 1H), | | |
| | 1-[3-(trifluoromethyl)- | 8.03-7.72 (m, 4H), 7.58 (d, J = 8.2 Hz, | | |
| | phenyl]-1,2-dihydropyridine- | 1H), 4.74 (d, J = 5.1 Hz, 2H), | | |
| | 3-carboxamide | 3.29 (s, 3H), 2.78 (d, J = 3.3 Hz, | | |
| | | 3H), 2.08 (d, J = 2.7 Hz, 3H) | | |
| 8 | N-Cyclopropyl-5-[(3- | 9.07 (s, 1H), 8.77 (d, J = 9.1 Hz, | 491.1 | SM3 |
| | methoxyphenyl)sulfinyl]-6- | 1H), 7.84 (d, J = 7.6 Hz, 1H), | | |
| | methyl-2-oxo-1-[3- | 7.77 (q, J = 7.8 Hz, 1H), 7.49-7.39 (m, | | |
| | (trifluoromethyl)phenyl]-1,2- | 3H), 7.30 (dd, J = 4.4, 1.9 Hz, 1H), | | |
| | dihydropyridine-3- | 7.10 (d, J = 7.1 Hz, 1H), 7.04 (d, J = 7.9 Hz, | | |
| | carboxamide | 1H), 3.93 (s, 3H), | | |
| | | 2.90-2.87 (m, 1H), 2.39 (d, J = 7.2 Hz, 3H), | | |
| | | 0.77 (d, J = 7.2 Hz, 2H), 0.51 (d, J = 3.5 Hz, | | |
| | | 2H) | | |
| 9 | N-Cyclopropyl-5-[(2- | 9.16 (s, 1H), 8.70 (d, J = 6.1 Hz, | 491.2 | SM3 |
| | methoxyphenyl)sulfinyl]-6- | 1H), 8.07 (dt, J = 7.7, 1.9 Hz, 1H), | | |
| | methyl-2-oxo-1-[3- | 7.83 (t, J = 7.4 Hz, 1H), 7.76 (m, | | |
| | (trifluoromethyl)phenyl]-1,2- | 1H), 7.55-7.48 (m, 2H), 7.37 (d, J = 7.7 Hz, | | |
| | dihydropyridine-3- | 1H), 7.31 (dt, J = 7.7, 1.3 Hz, | | |
| | carboxamide | 1H), 6.93-6.90 (m, 1H), | | |
| | | 3.83 (d, J = 3.7 Hz, 3H), 2.91-2.84 (m, | | |
| | | 1H), 2.41 (d, J = 2.2 Hz, 3H), | | |
| | | 0.79-0.72 (m, 2H), 0.55-0.46 (m, 2H) | | |
| 10 | 5-[(4-Cyanophenyl)sulfinyl]- | 9.35 (s, 1H), 8.66 (d, J = 12.5 Hz, | 504.1 | SM6 |
| | N-[(2S)-2-hydroxypropyl]-6- | 1H), 7.88-7.81 (m, 3H), | | |
| | methyl-2-oxo-1-[3- | 7.81-7.77 (m, 3H), 7.51 (t, J = 7.7 Hz, 1H), | | |
| | (trifluoromethyl)phenyl]-1,2- | 7.42 (t, J = 8.7 Hz, 1H), 3.95 (m, | | |
| | dihydropyridine-3- | 1H), 3.51-3.47 (m, 1H), | | |
| | carboxamide | 3.34-3.28 (m, 1H), 2.42 (d, J = 7.3 Hz, 3H), | | |
| | | 1.18 (d, J = 6.3 Hz, 3H) | | |
| 11 | 5-[(4-Cyanophenyl)sulfinyl]- | 8.41 (s, 2H), 8.31 (d, J = 2.6 Hz, | 446.0 | SM7 |
| | 6-methyl-2-oxo-1-[3- | 1H), 8.10 (d, J = 8.1 Hz, 2H), | | |
| | (trifluoromethyl)phenyl]-1,2- | 8.00 (d, J = 5.6 Hz, 1H), 7.97-7.72 (m, | | |
| | dihydropyridine-3- | 6H), 2.34 (s, 3H) | | |
| | carboxamide | | | |
| 12 | 5-[(4-Cyanophenyl)sulfinyl]- | 9.63 (t, J = 6.0 Hz, 1H), 8.32 (s, 1H), | 614.0 | SM1 |
| | 6-methyl-N-[4- | 8.10 (d, J = 8.5 Hz, 2H), 8.01 (d, J = 8.2 Hz, | | |
| | (methylsulfonyl)benzyl]-2- | 1H), 7.97-7.77 (m, 7H), | | |
| | oxo-1-[3-(trifluoromethyl)- | 7.49 (d, J = 8.2 Hz, 2H), 4.53 (d, J = 6.0 Hz, | | |
| | phenyl]-1,2-dihydropyridine- | 2H), 3.15 (s, 3H), 2.36 (s, | | |
| | 3-carboxamide | 3H) | | |
| 13 | 5-[(2-Cyanoethyl)sulfinyl]-6- | 9.75 (t, J = 6.1 Hz, 1H), 8.70 (d, J = 1.1 Hz, | 566.4 | SM1 |
| | methyl-N-[4- | 1H), 7.99-7.76 (m, 5H), | | |
| | (methylsulfonyl)benzyl]-2- | 7.70 (d, J = 7.9 Hz, 1H), 7.55 (d, J = 8.4 Hz, | | |
| | oxo-1-[3-(trifluoromethyl)- | 2H), 4.67-4.51 (m, 2H), | | |
| | phenyl]-1,2-dihydropyridine- | 3.41-3.19 (m, 2H), 3.18 (s, 3H), | | |
| | 3-carboxamide | 3.02-2.77 (m, 2H), 2.09 (d, J = 2.2 Hz, | | |
| | | 3H) | | |

### EXAMPLE 14

### 5-[(4-Cyanophenyl)sulfinyl]-N-cyclopropyl-1-(3,5-difluorophenyl)-6-methyl-2-oxo-1,2-dihydropyridine-3-carboxamide

To a mixture of N-cyclopropyl-1-(3,5-difluorophenyl)-5-iodo-6-methyl-2-oxo-1,2-dihydropyridine-3-carboxamide (SM8, 25 mg, 0.058 mmol), copper(I) iodide (1.9 mg, 0.01 mmol) and (±)-trans-cyclohexane-1,2-diamine (1.14 mg, 0.01 mmol) in acetonitrile (1.5 ml), 4-mercaptobenzonitrile (10 mg, 0.075 mmol) was added and the mixture was heated in a microwave reactor to 90° C. for 15 min. The residue obtained on evaporation was then diluted with water (15 ml) and extracted with ethyl acetate. The organic phase was dried (MgSO₄), filtered and evaporated. To the residue dissolved in acetic acid (1 ml) was added hydrogen peroxide (35% in water, 0.10 ml). The mixture was stirred overnight at room temperature. The compound was then purified on an Xterra C8 column using a gradient of acetonitrile/water. Freeze drying of the collected fractions afforded the title compound (3 mg, 7%).

¹H NMR (300 MHz, CDCl₃) δ 8.98 (t, J=3.5 Hz, 1H), 8.64 (s, 1H), 7.86 (dd, J=6.8, 1.8 Hz, 2H), 7.77 (dd, J=6.7, 1.7 Hz, 2H), 7.10-7.03 (m, 1H), 6.83-6.78 (m, 2H), 2.92-2.86 (m, 1H), 2.48 (s, 3H), 0.80-0.76 (m, 2H), 0.54-0.49 (m, 2H); APCI-MS m/z: 454.0 [MH⁺].

### EXAMPLES 15 TO 43

The following compounds were synthesised using an analogous method to that described for Example 14.

| Ex. | Compound | ¹H NMR | m/z | SM |
|---|---|---|---|---|
| 15 | 5-[(4-Cyanophenyl)sulfinyl]- | 10.05 (t, J = 5.3 Hz, 1H), 9.03 (d, J = 1.7 Hz, | 615.5 | SM9 |
| | N-{[5-(ethylsulfonyl)pyridin- | 1H), 8.54 (d, J = 2.7 Hz, 1H), | | |
| | 2-yl]methyl}-6-methyl-2- | 8.13 (dd, J = 5.9, 2.5 Hz, 2H), 7.85 (d, | | |
| | oxo-1-[3-(trifluoromethyl)- | J = 11.0 Hz, 5H), 7.75 (t, J = 7.9 Hz, | | |
| | phenyl]-1,2-dihydropyridine- | 1H), 7.70 (s, 1H), 7.62 (d, J = 8.2 Hz, | | |
| | 3-carboxamide | 1H), 7.48 (d, J = 8.2 Hz, 1H), 4.83 (d, | | |
| | | J = 5.5 Hz, 2H), 3.13 (q, J = 7.4 Hz, | | |
| | | 2H), 1.32-1.25 (m, 3H) | | |
| 16 | 5-[(4-Cyanophenyl)sulfinyl]- | 9.85 (t, 1H), 9.03 (s, 1H), 8.65 (s, 1H), | 597.2 | SM10 |
| | 1-(3,5-difluorophenyl)-N-{[5- | 8.16 (d, J = 7.9 Hz, 1H), 7.86 (d, J = 8.3 Hz, | | |
| | (ethylsulfonyl)pyridin-2- | 2H), 7.78 (d, J = 8.3 Hz, 2H), | | |
| | yl]methyl}-6-methyl-2-oxo- | 7.52 (d, J = 8.3 Hz, 1H), 7.08 (t, J = 8.6 Hz, | | |
| | 1,2-dihydropyridine-3- | 1H), 6.83 (t, J = 6.9 Hz, 2H), | | |
| | carboxamide | 4.83 (d, 2H), 3.14 (q, J = 7.4 Hz, 2H), | | |
| | | 2.50 (s, 3H), 1.32-1.26 (m, 3H) | | |
| 17 | 5-[(4-Cyanophenyl)sulfinyl]- | 9.79 (t, 1H), 8.92 (d, J = 1.7 Hz, 1H), | 629.2 | SM11 |
| | 1-(3,5-dichlorophenyl)-N- | 8.32 (s, 1H), 8.09 (d, J = 8.3 Hz, 2H), | | |
| | {[5-(ethylsulfonyl)pyridin-2- | 7.89 (d, J = 8.1 Hz, 2H), 7.75 (d, J = 7.4 Hz, | | |
| | yl]methyl}-6-methyl-2-oxo- | 2H), 7.54 (d, J = 8.3 Hz, 1H), | | |
| | 1,2-dihydropyridine-3- | 4.68 (d, J = 4.5 Hz, 3H), | | |
| | carboxamide | 3.37-3.32 (m, 2H), 2.43 (s, 3H), 1.10 (t, J = 7.3 Hz, | | |
| | | 3H) | | |
| 18 | 5-[(4-Cyanophenyl)sulfinyl]- | 9.06 (d, J = 4.8 Hz, 1H), 8.86 (d, J = 2.8 Hz, | 446.2 | SM12 |
| | N,6-dimethyl-2-oxo-1-[3- | 1H), 8.23 (d, J = 2.6 Hz, 1H), | | |
| | (trifluoromethyl)phenyl]-1,2- | 8.10 (quintet, J = 2.1 Hz, 3H), | | |
| | dihydropyridine-3- | 7.97-7.92 (m, 4H), 7.84 (d, J = 7.9 Hz, 1H), | | |
| | carboxamide | 2.76 (d, J = 4.8 Hz, 3H) | | |
| 19 | 5-[(4-Cyanophenyl)sulfinyl]- | 8.95 (d, J = 4.7 Hz, 1H), 8.28 (s, 1H), | | SM13 |
| | 1-(3,5-dichlorophenyl)-N,6- | 8.09 (d, J = 8.3 Hz, 2H), | | |
| | dimethyl-2-oxo-1,2- | 7.90-7.86 (m, 3H), 7.72 (dd, J = 4.1, 1.6 Hz, | | |
| | dihydropyridine-3- | 2H), 2.74 (d, J = 4.8 Hz, 3H), 2.41 (s, | | |
| | carboxamide | 3H) | | |
| 20 | 5-[(4-Cyanophenyl)sulfinyl]- | 9.18 (t, J = 6.0 Hz, 1H), 8.89 (s, 1H), | 492.1 | SM14 |
| | 1-(3,5-difluorophenyl)-N-[2- | 8.29 (s, 1H), 8.10 (dt, J = 8.5, 1.8 Hz, | | |
| | (1H-imidazol-4-yl)ethyl]-6- | 2H), 7.88 (dt, J = 8.5, 1.7 Hz, 2H), | | |
| | methyl-2-oxo-1,2- | 7.57-7.49 (m, 1H), 7.40-7.36 (m, | | |
| | dihydropyridine-3- | 3H), 3.55 (q, J = 6.4 Hz, 2H), 2.84 (t, | | |
| | carboxamide | J = 6.6 Hz, 2H), 2.42 (s, 3H) | | |
| 21 | 5-[(4-Cyanophenyl)sulfinyl]- | | 527.1 | SM15 |
| | 1-(3,5-difluorophenyl)-6- | | | |
| | methyl-N-(2-morpholin-4- | | | |
| | ylethyl)-2-oxo-1,2-dihydro- | | | |
| | pyridine-3-carboxamide | | | |
| 22 | 5-[(4-Cyanophenyl)sulfinyl]- | 9.01 (d, J = 4.0 Hz, 1H), 8.64 (s, 1H), | 428.2 | SM16 |
| | 1-(3,5-difluorophenyl)-N,6- | 7.86 (d, J = 8.5 Hz, 2H), 7.78 (d, J = 8.6 Hz, | | |
| | dimethyl-2-oxo-1,2-dihydro- | 2H), 7.07 (tt, J = 8.6, 2.2 Hz, | | |
| | pyridine-3-carboxamide | 1H), 6.82-6.79 (m, 2H), 2.91 (d, J = 5.0 Hz, | | |
| | | 3H), 2.49 (s, 3H) | | |
| 23 | 5-[(4-Cyanophenyl)sulfinyl]- | 9.55 (t, J = 5.3 Hz, 1H), 8.10 (d, J = 7.5 Hz, | 541.0 | SM17 |
| | 6-methyl-N-[(3- | 1H), 8.01 (d, J = 7.8 Hz, 2H), | | |
| | methylisoxazol-5-yl)methyl]- | 7.87 (m, 6H), 6.11 (s, 1H), 4.53 (m, | | |
| | 2-oxo-1-[3-(trifluoromethyl)- | Hz, 2H), 2.36 (d, J = 2.3 Hz, 3H), | | |
| | phenyl]-1,2-dihydropyridine- | 2.16 (d, J = 9.7 Hz, 3H) | | |
| | 3-carboxamide | | | |
| 24 | N-Cyclopropyl-5-[(4- | 9.39 (s, 1H), 8.87 (d, J = 7.3 Hz, 1H), | 477.1 | SM3 |
| | hydroxyphenyl)sulfinyl]-6- | 7.87-7.74 (m, 2H), 7.59-7.54 (m, | | |
| | methyl-2-oxo-1-[3- | 2H), 7.50 (d, J = 7.3 Hz, 2H), 7.42 (t, | | |
| | (trifluoromethy)phenyl]-1,2- | J = 7.7 Hz, 1H), 7.11-7.07 (m, 1H), | | |
| | dihydropyridine-3- | 2.88 (dq, J = 7.2, 3.7 Hz, 2H), 2.42 (d, | | |
| | carboxamide | J = 4.2 Hz, 2H), 0.81 (dt, J = 8.5, 1.2 Hz, | | |
| | | 2H), 0.54 (dd, J = 10.0, 4.1 Hz, | | |
| | | 2H) | | |
| 25 | 5-[(4-Cyanophenyl)sulfinyl]- | 9.20 (s, 1H), 9.07 (s, 1H), 8.30 (d, J = 2.3 Hz, | 554.2 | SM18 |
| | N-[3-(1H-imidazol-1- | 1H), 8.11 (d, J = 8.0 Hz, 2H), | | |
| | yl)propyl]-6-methyl-2-oxo-1- | 8.02-7.73 (m, 7H), 7.65 (s, 1H), | | |
| | [3-(trifluoromethyl)phenyl]- | 4.15 (t, J = 7.0 Hz, 2H), 3.24 (q, J = 6.4 Hz, | | |
| | 1,2-dihydropyridine-3- | 2H), 2.37 (s, 3H), 1.99 (quintet, J = 6.9 Hz, | | |
| | carboxamide | 2H) | | |
| 26 | 5-[(4-Cyanophenyl)sulfinyl]- | 9.20 (s, 1H), 8.31 (d, J = 2.5 Hz, 1H), | 555.4 | SM19 |
| | 6-methyl-2-oxo-N-[3-(1H- | 8.13-8.08 (m, 3H), 8.03-7.78 (m, | | |
| | 1,2,3-triazol-1-yl)propyl]-1- | 6H), 7.68 (d, J = 0.9 Hz, 1H), 4.34 (t, | | |
| | [3-(trifluoromethyl)phenyl]- | J = 7.0 Hz, 2H), 3.28-3.16 (m, 2H), | | |
| | 1,2-dihydropyridine-3- | 2.36 (s, 3H), 1.99 (quintet, J = 6.9 Hz, | | |
| | carboxamide | 2H) | | |
| 27 | 5-[(4-Cyanophenyl)sulfinyl]- | 9.35 (t, J = 5.1 Hz, 1H), 8.32 (d, J = 1.5 Hz, | 516.2 | SM20 |
| | N-[(1-hydroxycyclopropyl)- | 1H), 8.13-7.78 (m, 9H), | | |
| | methyl]-6-methyl-2-oxo-1-[3- | 3.31 (m, 2H), 2.35 (s, 3H), 0.57-0.38 (m, | | |
| | (trifluoromethyl)phenyl]-1,2- | 4H) | | |
| | dihydropyridine-3- | | | |
| | carboxamide | | | |
| 28 | 1-(3-Cyanophenyl)-5-[(4- | δ 9.80 (t, J = 5.9 Hz, 1H), 8.96 (d, J = 1.8 Hz, | 572.2 | SM5 |
| | cyanophenyl)sulfinyl]-6- | 1H), 8.34 (d, J = 5.8 Hz, 1H), | | |
| | methyl-N-{[5- | 8.24 (dd, J = 8.2, 2.2 Hz, 1H), | | |
| | (methylsulfonyl)pyridin-2- | 8.13-8.04 (m, 4H), 7.92-7.81 (m, 4H), | | |
| | yl]methyl}-2-oxo-1,2- | 7.53 (d, J = 8.3 Hz, 1H), 4.57 (m, 2H), | | |
| | dihydropyridine-3- | 3.28 (s, 3H), 3.28 (s, 3H) | | |
| | carboxamide | | | |
| 29 | 5-[(4-Cyanophenyl)sulfinyl]- | 9.21 (s, 1H), 8.32 (d, J = 1.7 Hz, 1H), | 504.1 | SM21 |
| | N-(2-methoxyethyl)-6- | 8.24-7.78 (m, 8H), 3.46-3.30 (m, | | |
| | methyl-2-oxo-1-[3- | 4H), 3.19 (s, 3H), 2.35 (s, 3H) | | |
| | (trifluoromethyl)phenyl]-1,2- | | | |
| | dihydropyridine-3- | | | |
| | carboxamide | | | |
| 30 | 5-[(4-Cyanophenyl)sulfinyl]- | 9.27 (t, J = 5.4 Hz, 1H), 8.33 (d, J = 1.5 Hz, | 518.5 | SM22 |
| | N-(2-hydroxy-2- | 1H), 8.10 (d, J = 8.6 Hz, 2H), | | |
| | methylpropyl)-6-methyl-2- | 8.02 (s, 1H), 7.98-7.78 (m, 6H), | | |
| | oxo-1-[3-(trifluoromethyl)- | 3.21-3.14 (m, 2H), 2.34 (s, 3H), 1.04 (s, | | |
| | phenyl]-1,2-dihydropyridine- | 3H), 1.02 (s, 3H) | | |
| | 3-carboxamide | | | |
| 31 | 5-[(4-Chlorophenyl)sulfinyl]- | δ 9.66 (t, J = 6.0 Hz, 1H), 8.39 (d, J = 0.9 Hz, | 623.3, 625.3 | SM1 |
| | 6-methyl-N-[4- | 1H), 8.01 (d, J = 5.7 Hz, 1H), | | |
| | (methylsulfonyl)benzyl]-2- | 7.93 (d, J = 7.9 Hz, 1H), | | |
| | oxo-1-[3-(trifluoromethyl)- | 7.89-7.66 (m, 8H), 7.50 (d, J = 8.4 Hz, 2H), | | |
| | phenyl]-1,2-dihydropyridine- | 4.54 (dd, J = 8.4, 5.6 Hz, 2H), 3.16 (s, 3H), | | |
| | 3-carboxamide | 2.32 (s, 3H) | | |
| 32 | 6-Methyl-5-[(4- | 9.67 (t, J = 6.1 Hz, 1H), 8.43 (d, J = 2.3 Hz, | 603.1 | SM1 |
| | methylphenyl)sulfinyl]-N-[4- | 1H), 8.01 (s, 1H), 7.93 (d, J = 6.9 Hz, | | |
| | (methylsulfonyl)benzyl]-2- | 1H), 7.89-7.77 (m, 4H), | | |
| | oxo-1-[3-(trifluoromethyl)- | 7.59 (dd, J = 6.5, 1.7 Hz, 2H), 7.50 (d, J = 8.2 Hz, | | |
| | phenyl]-1,2-dihydropyridine- | 2H), 7.42 (d, J = 8.5 Hz, 2H), | | |
| | 3-carboxamide | 4.54 (m, 2H), 3.15 (s, 3H), 2.37 (s, | | |
| | | 3H), 2.31 (d, J = 0.5 Hz, 3H) | | |
| 33 | 6-Methyl-N-[4- | 9.63 (t, J = 6.1 Hz, 1H), 8.42 (dd, J = 9.0, | 634.0 | SM1 |
| | (methylsulfonyl)benzyl]-5- | 0.9 Hz, 2H), 8.35 (d, J = 1.1 Hz, | | |
| | [(4-nitrophenyl)sulfinyl]-2- | 1H), 8.05-7.77 (m, 8H), 7.49 (d, J = 8.4 Hz, | | |
| | oxo-1-[3-(trifluoromethyl)- | 2H), 4.52 (m, 2H), 3.15 (s, | | |
| | phenyl]-1,2-dihydropyridine- | 3H), 2.38 (s, 3H) | | |
| | 3-carboxamide | | | |
| 34 | 6-Methyl-N-[4- | 9.64 (t, J = 6.1 Hz, 1H), 8.37 (s, 1H), | 657.0 | SM1 |
| | (methylsulfonyl)benzyl]-2- | 8.04-7.77 (m, 10H), 7.49 (d, J = 8.2 Hz, | | |
| | oxo-1-[3-(trifluoromethyl)- | 2H), 4.53 (m, 2H), 3.15 (s, 3H), | | |
| | phenyl]-5-{[4- | 2.37 (s, 3H) | | |
| | (trifluoromethyl)phenyl]sulfinyl}- | | | |
| | 1,2-dihydropyridine-3- | | | |
| | carboxamide | | | |
| 35 | 5-{[4-(Acetylamino)phenyl]- | 10.26 (s, 1H), 9.68 (t, J = 6.0 Hz, 1H), | 646.1 | SM1 |
| | sulfinyl}-6-methyl-N-[4- | 8.46 (d, J = 2.9 Hz, 1H), 8.01 (s, 1H), | | |
| | (methylsulfonyl)benzyl]-2- | 7.93 (d, J = 7.0 Hz, 1H), | | |
| | oxo-1-[3-(trifluoromethyl)- | 7.88-7.75 (m, 6H), 7.62 (d, J = 8.7 Hz, 2H), | | |
| | phenyl]-1,2-dihydropyridine- | 7.50 (d, J = 8.1 Hz, 2H), 4.54 (m, 2H), | | |
| | 3-carboxamide | 3.15 (s, 3H), 2.28 (d, J = 1.7 Hz, 3H), | | |
| | | 2.07 (s, 3H) | | |
| 36 | 5-[(4-Ethylphenyl)sulfinyl]- | 9.68 (t, J = 6.1 Hz, 1H), 8.45 (d, J = 1.7 Hz, | 617.1 | SM1 |
| | 6-methyl-N-[4- | 1H), 8.02 (s, 1H), 7.93 (d, J = 6.5 Hz, | | |
| | (methylsulfonyl)benzyl]-2- | 1H), 7.89-7.76 (m, 4H), | | |
| | oxo-1-[3-(trifluoromethyl)- | 7.61 (d, J = 8.2 Hz, 2H), 7.53-7.41 (m, | | |
| | phenyl]-1,2-dihydropyridine- | 4H), 4.54 (m, 2H), 3.15 (s, 3H), | | |
| | 3-carboxamide | 2.68 (q, J = 7.6 Hz, 2H), 2.31 (d, J = 0.7 Hz, | | |
| | | 3H), 1.19 (t, J = 7.6 Hz, 3H) | | |
| 37 | 5-[(4-Fluorophenyl)sulfinyl]- | 9.67 (t, J = 6.0 Hz, 1H), 8.42 (d, J = 1.4 Hz, | 607.3 | SM1 |
| | 6-methyl-N-[4- | 1H), 8.01 (d, J = 6.9 Hz, 1H), | | |
| | (methylsulfonyl)benzyl]-2- | 7.93 (d, J = 7.1 Hz, 1H), | | |
| | oxo-1-[3-(trifluoromethyl)- | 7.89-7.73 (m, 6H), 7.54-7.43 (m, 4H), 4.23 (m, | | |
| | phenyl]-1,2-dihydropyridine- | 2H), 3.16 (s, 3H), 2.32 (s, 3H) | | |
| | 3-carboxamide | | | |
| 38 | 5-[(4-Cyanophenyl)sulfinyl]- | 9.90 (t, J = 5.6 Hz, 1H), 8.96 (d, J = 1.7 Hz, | 561.1 | SM5 |
| | 6-methyl-1-(3- | 1H), 8.31 (s, 1H), 8.24 (dd, J = 8.2, | | |
| | methylphenyl)-N-{[5- | 2.3 Hz, 1H), 8.09 (dd, J = 8.5, | | |
| | (methylsulfonyl)pyridin-2- | 1.2 Hz, 2H), 7.90 (dd, J = 8.4, 1.4 Hz, | | |
| | yl]methyl}-2-oxo-1,2- | 2H), 7.57-7.45 (m, 2H), 7.37 (d, J = 7.7 Hz, | | |
| | dihydropyridine-3- | 1H), 7.28-7.21 (m, 2H), | | |
| | carboxamide | 4.67 (d, J = 4.4 Hz, 2H), 3.28 (s, 3H), | | |
| | | 2.38 (s, 3H), 2.38 (s, 3H) | | |
| 39 | 5-[(4-Cyanophenyl)sulfinyl]- | 9.08 (t, J = 5.6 Hz, 1H), 8.31 (d, J = 1.5 Hz, | 474.0 | SM23 |
| | N-ethyl-6-methyl-2-oxo-1-[3- | 1H), 8.10 (d, J = 7.8 Hz, 2H), | | |
| | (trifluoromethyl)phenyl]-1,2- | 8.04-7.76 (m, 6H), 3.23 (ddd, J = 20.1, | | |
| | dihydropyridine-3- | 7.3, 2.1 Hz, 2H), 2.35 (s, 3H), | | |
| | carboxamide | 1.03 (t, J = 7.2 Hz, 3H) | | |
| 40 | 5-[(4-Chlorophenyl)sulfinyl]- | 9.01 (d, J = 4.8 Hz, 1H), 8.36 (d, J = 2.0 Hz, | 469.3, 471.2 | SM24 |
| | N,6-dimethyl-2-oxo-1-[3- | 1H), 8.02-7.65 (m, 8H), | | |
| | (trifluoromethyl)phenyl]-1,2- | 2.74 (d, J = 4.8 Hz, 3H), 2.32 (s, 3H) | | |
| | dihydropyridine-3- | | | |
| | carboxamide | | | |
| 41 | N-Ethyl-5-[(4- | 9.12 (t, J = 5.4 Hz, 1H), 8.40 (d, J = 1.9 Hz, | 467.3 | SM23 |
| | fluorophenyl)sulfinyl]-6- | 1H), 8.04-7.72 (m, 6H), | | |
| | methyl-2-oxo-1-[3- | 7.53-7.43 (m, 2H), 3.24 (m, 2H), 2.30 (d, | | |
| | (trifluoromethyl)phenyl]-1,2- | J = 0.7 Hz, 3H), 1.04 (t, J = 7.2 Hz, | | |
| | dihydropyridine-3- | 3H) | | |
| | carboxamide | | | |
| 42 | 5-[(4-Fluorophenyl)sulfinyl]- | 9.02 (d, J = 4.4 Hz, 1H), 8.39 (d, J = 3.0 Hz, | 453.2 | SM24 |
| | N,6-dimethyl-2-oxo-1-[3- | 1H), 8.03-7.73 (m, 6H), | | |
| | (trifluoromethyl)phenyl]-1,2- | 7.52-7.43 (m, 2H), 2.75 (d, J = 4.4 Hz, | | |
| | dihydropyridine-3- | 3H), 2.31 (s, 3H) | | |
| | carboxamide | | | |
| 43 | 5-[(4-Bromophenyl)sulfinyl]- | 9.01 (q, J = 4.6 Hz, 1H), 8.36 (d, J = 2.5 Hz, | 515.2, 513.2 | SM24 |
| | N,6-dimethyl-2-oxo-1-[3- | 1H), 7.99 (d, J = 4.4 Hz, 1H), | | |
| | (trifluoromethyl)phenyl]-1,2- | 7.93 (d, J = 7.6 Hz, 1H), | | |
| | dihydropyridine-3- | 7.88-7.76 (m, 4H), 7.65 (d, J = 8.5 Hz, 2H), | | |
| | carboxamide | 2.74 (d, J = 4.2 Hz, 3H), 2.31 (s, 3H) | | |

### EXAMPLE 44

### 5-[(4-Cyanophenyl)sulfinyl]-N-(2-hydroxyethyl)-6-methyl-2-oxo-1-[3-(trifluoromethyl)phenyl]-1,2-dihydropyridine-3-carboxamide

To a mixture of 5-[(4-cyanophenyl)sulfinyl]-6-methyl-2-oxo-1-[3-(trifluoromethyl)phenyl]-1,2-dihydropyridine-3-carboxylic acid (SM25, 25 mg, 0.056 mmol) and HATU (21 mg, 0.056 mmol) in NMP (1 ml) was added ethanol amine (7 mg, 0.12 mmol) and DIPEA (0.12 mmol). The reaction was heated in a microwave reactor to 50° C. for 10 min. The crude compound was then purified on an Xterra C8 column using a gradient of acetonitrile/water. Freeze drying afforded the title compound (1 mg, 4%).

¹H NMR (400 MHz, DMSO-d₆): δ 11.84 (s, 1H), 8.42 (s, 1H), 8.25 (s, 1H), 8.07 (d, J=8.5 Hz, 2H), 7.86 (d, J=8.5 Hz, 2H), 7.80 (d, J=8.9 Hz, 1H), 7.59 (t, J=8.0 Hz, 1H), 7.47 (d, J=7.3 Hz, 1H), 5.09 (t, J=5.4 Hz, 1H), 4.30-4.24 (m, 2H), 3.76 (d, J=5.5 Hz, 2H), 2.93 (s, 3H);

APCI-MS m/z: 490.0 [MH].

### EXAMPLES 45 TO 55

The following compounds were synthesised using an analogous method to that described for Example 44.

| Ex. | Compound | ¹H NMR | m/z | SM |
|---|---|---|---|---|
| 45 | 5-[(4-Cyanophenyl)sulfinyl]- | 9.09 (s, 1H), 8.66 (d, J = 9.5 Hz, | 500.1 | SM25 |
| | N-(cyclopropylmethyl)-6- | 1H), 7.88-7.76 (m, 6H), 7.51 (t, J = 2.1 Hz, | | |
| | methyl-2-oxo-1-[3- | 1H), 7.44 (t, J = 6.8 Hz, 1H), | | |
| | (trifluoromethyl)phenyl]-1,2- | 3.22 (ddd, J = 7.1, 5.5, 1.8 Hz, 2H), | | |
| | dihydropyridine-3- | 2.41 (d, J = 10.1 Hz, 3H), | | |
| | carboxamide | 1.00-0.91 (m, 1H), 0.47 (dt, J = 7.8, 5.0 Hz, | | |
| | | 2H), 0.19 (dd, J = 14.7, 1.3 Hz, 2H) | | |
| 46 | N-Methyl-2-oxo-5- | 9.08 (q, J = 5.0 Hz, 1H), 8.78 (d, J = 2.6 Hz, | 421.0 | SM26 |
| | (phenylsulfinyl)-1-[3- | 1H), 8.26 (d, J = 2.6 Hz, | | |
| | (trifluoromethyl)phenyl]-1,2- | 1H), 8.10 (s, 1H), 7.97-7.89 (m, | | |
| | dihydropyridine-3- | 2H), 7.86-7.72 (m, 3H), | | |
| | carboxamide | 7.66-7.53 (m, 3H), 2.76 (d, J = 4.8 Hz, 3H) | | |
| 47 | N-(Cyanomethyl)-5-[(4- | 9.51 (t, J = 5.8 Hz, 1H), 8.34 (s, 1H), | 485.0 | SM25 |
| | cyanophenyl)sulfinyl]-6- | 8.11 (d, J = 8.1 Hz, 2H), | | |
| | methyl-2-oxo-1-[3- | 8.02-7.82 (m, 6H), 4.24 (d, J = 5.9 Hz, 2H), | | |
| | (trifluoromethyl)phenyl]-1,2- | 2.36 (s, 3H) | | |
| | dihydropyridine-3- | | | |
| | carboxamide | | | |
| 48 | 5-[(4-Cyanophenyl)sulfinyl]- | 9.20 (t, J = 5.0 Hz, 1H), 8.94 (d, J = 1.1 Hz, | 540.1 | SM25 |
| | N-[2-(1H-imidazol-4- | 1H), 8.29 (d, J = 2.2 Hz, | | |
| | yl)ethyl]-6-methyl-2-oxo-1- | 1H), 8.10 (d, J = 8.0 Hz, 2H), | | |
| | [3-(trifluoromethyl)phenyl]- | 7.99 (d, J = 5.1 Hz, 6H), 7.89 (m, 1H), | | |
| | 1,2-dihydropyridine-3- | 7.40 (s, 2H), 2.85 (t, J = 6.7 Hz, 2H), | | |
| | carboxamide | 2.35 (s, 3H) | | |
| 49 | 5-[(4-Cyanophenyl)sulfinyl]- | 9.24 (t, J = 5.6 Hz, 1H), 8.32 (t, J = 1.7 Hz, | 504.1 | SM25 |
| | N-(2-hydroxypropyl)-6- | 1H), 8.12-8.08 (m, 2H), | | |
| | methyl-2-oxo-1-[3- | 8.00 (d, J = 4.8 Hz, 1H), | | |
| | (trifluoromethyl)phenyl]-1,2- | 7.96-7.80 (m, 5H), 4.77-4.73 (m, 1H), | | |
| | dihydropyridine-3- | 3.68-3.62 (m, 1H), 3.13-3.01 (m, 1H), | | |
| | carboxamide | 2.35 (s, 3H), 0.99 (dd, J = 6.2, 2.0 Hz, | | |
| | | 3H) | | |
| 50 | 5-[(4-Cyanophenyl)sulfinyl]- | 9.16 (t, J = 2.0 Hz, 1H), 8.30 (d, J = 2.6 Hz, | 559.1 | SM25 |
| | 6-methyl-N-(2-morpholin-4- | 1H), 8.10 (dd, J = 8.5, 1.5 Hz, | | |
| | ylethyl)-2-oxo-1-[3- | 2H), 8.01 (m, 1H), 7.95 (d, J = 7.6 Hz, | | |
| | (trifluoromethyl)phenyl]-1,2- | 1H), 7.91-7.80 (m, 4H), | | |
| | dihydropyridine-3- | 3.49 (t, J = 4.5 Hz, 4H), 3.34 (m, | | |
| | carboxamide | 2H), 2.38-2.32 (m, 9H) | | |
| 51 | 5-[(4-Cyanophenyl)sulfinyl]- | 9.30 (s, 1H), 8.67 (d, J = 17.3 Hz, | 518.1 | SM25 |
| | N-(2-hydroxy-1,1- | 1H), 7.87 (dd, J = 8.4, 4.2 Hz, 3H), | | |
| | dimethylethyl)-6-methyl-2- | 7.49 (d, J = 10.1 Hz, 1H), | | |
| | oxo-1-[3-(trifluoromethyl)- | 7.45-7.40 (m, 2H), 3.60 (d, J = 10.8 Hz, 2H), | | |
| | phenyl]-1,2-dihydropyridine- | 3.61 (s, 2H), 2.41 (d, J = 6.7 Hz, | | |
| | 3-carboxamide | 3H), 1.32-1.30 (m, 6H) | | |
| 52 | 5-[(4-Cyanophenyl)sulfinyl]- | 8.98 (s, 1H), 8.65 (d, J = 12.6 Hz, | 460.0 | SM25 |
| | N,6-dimethyl-2-oxo-1-[3- | 1H), 7.87-7.84 (m, 3H), 7.79 (m, | | |
| | (trifluoromethyl)phenyl]-1,2- | 3H), 7.50 (d, J = 5.8 Hz, 1H), | | |
| | dihydropyridine-3- | 7.42 (t, J = 7.8 Hz, 1H), 2.90 (dd, J = 4.8, | | |
| | carboxamide | 2.3 Hz, 3H), 2.42 (d, J = 7.8 Hz, 3H) | | |
| 53 | 5-[(4-Cyanophenyl)sulfinyl]- | 9.35 (s, 1H), 8.66 (d, J = 12.5 Hz, | 504.1 | SM25 |
| | N-[(2R)-2-hydroxypropyl]-6- | 1H), 7.88-7.81 (m, 3H), | | |
| | methyl-2-oxo-1-[3- | 7.81-7.77 (m, 3H), 7.51 (t, J = 7.7 Hz, 1H), | | |
| | (trifluoromethyl)phenyl]-1,2- | 7.42 (t, J = 8.7 Hz, 1H), 3.95 (m, | | |
| | dihydropyridine-3- | 1H), 3.51-3.47 (m, 1H), | | |
| | carboxamide | 3.34-3.28 (m, 1H), 2.42 (d, J = 7.3 Hz, 3H), | | |
| | | 1.18 (d, J = 6.3 Hz, 3H) | | |
| 54 | 5-[(4-Cyanophenyl)sulfinyl]- | 9.15 (t, 1H), 8.30 (d, J = 2.0 Hz, 1H), | 571.1 | SM25 |
| | 6-methyl-2-oxo-N-[3-(2- | 8.10 (d, J = 7.2 Hz, 2H), 8.01 (d, J = 4.7 Hz, | | |
| | oxopyrrolidin-1-yl)propyl]-1- | 1H), 7.95-7.81 (m, 5H), | | |
| | [3-(trifluoromethyl)phenyl]- | 3.31-3.23 (m, 3H), 3.19-3.11 (m, | | |
| | 1,2-dihydropyridine-3- | 3H), 2.35 (s, 3H), 2.14 (t, J = 8.1 Hz, | | |
| | carboxamide | 2H), 1.86 (q, J = 7.5 Hz, 2H), | | |
| | | 1.61 (q, J = 6.9 Hz, 2H) | | |
| 55 | 5-[(4-Cyanophenyl)sulfinyl]- | 9.19 (t, J = 5.4 Hz, 1H), | 518.0 | SM25 |
| | N-(2-methoxypropyl)-6- | 8.33-8.31 (m, 1H), 8.13-7.78 (m, 8H), | | |
| | methyl-2-oxo-1-[3- | 3.48-3.31 (m, 2H), 3.29-3.11 (m, 4H), | | |
| | (trifluoromethyl)phenyl]-1,2- | 2.34 (s, 3H), 1.00 (m, 3H) | | |
| | dihydropyridine-3- | | | |
| | carboxamide | | | |

### EXAMPLE 56

### 6-Methyl-5-(methylsulfonyl)-N-{[5-(methylsulfonyl)pyridin-2-yl]methyl}-2-oxo-1-[3-(trifluoromethyl)phenyl]-1,2-dihydropyridine-3-carboxamide

From the reaction using starting material SM5 in which Example 7 was isolated, the title sulfone was also isolated (8 mg).

¹H NMR (399.99 MHz, DMSO-d₆) δ 9.82 (t, J=5.6 Hz, 1H), 8.99 (d, J=2.0 Hz, 1H), 8.79 (s, 1H), 8.27 (dd, J=8.2, 2.3 Hz, 1H), 8.00 (s, 1H), 7.96 (d, J=7.7 Hz, 1H), 7.88 (t, J=7.9 Hz, 1H), 7.81 (d, J=8.0 Hz, 1H), 7.57 (d, J=8.3 Hz, 1H), 4.73 (d, J=5.6 Hz, 2H), 3.29 (d, J=1.6 Hz, 6H), 2.39 (s, 3H);

APCI-MS m/z: 544.0 (MH⁺).

### EXAMPLES 57 TO 64

The following sulfone compounds were isolated from the previously described reactions yielding the corresponding sulfoxide compounds, or prepared analogously to Example 14.

| Ex. | Compound | ¹H NMR | m/z | SM |
|---|---|---|---|---|
| 57 | 2-Oxo-N-[3-(2-oxopyrrolidin- | 9.17 (t, J = 5.8 Hz, 1H), 8.83 (d, J = 2.9 Hz, | 548.1 | SM2 |
| | 1-yl)propyl]-5- | 1H), 8.50 (d, J = 2.9 Hz, | | |
| | (phenylsulfonyl)-1-[3- | 1H), 8.06 (m, 3H), 7.91 (t, J = 9.1 Hz, | | |
| | (trifluoromethyl)phenyl]-1,2- | 2H), 7.81 (t, J = 8.0 Hz, 1H), | | |
| | dihydropyridine-3- | 7.74 (t, J = 7.4 Hz, 1H), 7.67 (t, J = 7.6 Hz, | | |
| | carboxamide | 2H), 3.29 (t, J = 7.0 Hz, 2H), | | |
| | | 3.23 (q, J = 6.5 Hz, 2H), 3.16 (t, J = 6.8 Hz, | | |
| | | 2H), 2.16 (t, J = 8.1 Hz, 2H), | | |
| | | 1.87 (quintet, J = 7.5 Hz, 2H), | | |
| | | 1.64 (quintet, J = 6.9 Hz, 2H) | | |
| 58 | 5-[(4-Cyanophenyl)sulfonyl]- | 9.25 (s, 1H), 8.70 (s, 1H), 8.06 (d, J = 8.6 Hz, | 492.1 | SM7 |
| | N,6-dimethyl-2-oxo-1-[3- | 2H), 7.87 (dd, J = 14.6, | | |
| | (trifluoromethyl)phenyl]-1,2- | 8.2 Hz, 3H), 7.77 (t, J = 7.9 Hz, 1H), | | |
| | dihydropyridine-3- | 7.44 (s, 1H), 7.37 (d, J = 7.6 Hz, | | |
| | carboxamide | 1H), 5.83 (s, 1H), 2.36 (s, 3H) | | |
| 59 | 5-{[4-(Acetylamino)phenyl]- | 10.42 (s, 1H), 9.63 (t, J = 6.1 Hz, | 662.1 | SM1 |
| | sulfonyl}-6-methyl-N-[4- | 1H), 8.91 (s, 1H), 7.97 (s, 1H), | | |
| | (methylsulfonyl)benzyl]-2- | 7.92-7.72 (m, 9H), 7.54 (d, J = 8.3 Hz, | | |
| | oxo-1-[3-(trifluoromethyl)- | 2H), 4.59 (d, J = 6.2 Hz, 2H), | | |
| | phenyl]-1,2-dihydropyridine- | 3.17 (s, 3H), 2.22 (s, 3H), 2.08 (s, 3H) | | |
| | 3-carboxamide | | | |
| 60 | 5-[(4-Ethylphenyl)sulfonyl]- | 9.62 (t, J = 6.1 Hz, 1H), 8.91 (s, 1H), | 633.4 | SM1 |
| | 6-methyl-N-[4- | 7.98 (s, 1H), 7.92-7.75 (m, 7H), | | |
| | (methylsulfonyl)benzyl]-2- | 7.58-7.47 (m, 4H), 4.59 (d, J = 5.8 Hz, | | |
| | oxo-1-[3-(trifluoromethyl)- | 2H), 3.17 (s, 3H), 2.71 (q, J = 7.7 Hz, | | |
| | phenyl]-1,2-dihydropyridine- | 2H), 2.23 (s, 3H), 1.20 (t, J = 7.5 Hz, | | |
| | 3-carboxamide | 3H) | | |
| 61 | 5-[(4-Cyanophenyl)sulfonyl]- | 9.22 (s, 1H), 8.92 (d, J = 4.5 Hz, | 476.0 | SM24 |
| | N,6-dimethyl-2-oxo-1-[3- | 1H), 8.07 (d, J = 8.6 Hz, 2H), | | |
| | (trifluoromethyl)phenyl]-1,2- | 7.86 (dd, J = 13.0, 8.3 Hz, 3H), 7.76 (t, J = 7.9 Hz, | | |
| | dihydropyridine-3- | 1H), 7.43 (s, 1H), 7.36 (d, | | |
| | carboxamide | J = 8.0 Hz, 1H), 2.95 (d, J = 5.0 Hz, | | |
| | | 3H), 2.37 (s, 3H) | | |
| 62 | 5-[(4-Cyanophenyl)sulfonyl]- | 9.24 (d, J = 6.0 Hz, 2H), 8.07 (d, J = 8.4 Hz, | 534.1 | SM22 |
| | N-(2-hydroxy-1,1- | 2H), 7.87 (dd, J = 15.0, 8.2 Hz, | | |
| | dimethylethyl)-6-methyl-2- | 3H), 7.77 (t, J = 8.0 Hz, 1H), | | |
| | oxo-1-[3-(trifluoromethyl)- | 7.43 (s, 1H), 7.36 (d, J = 7.4 Hz, | | |
| | phenyl]-1,2-dihydropyridine- | 1H), 3.66 (d, J = 6.2 Hz, 2H), | | |
| | 3-carboxamide | 2.35 (s, 3H), 1.35 (d, J = 1.2 Hz, 6H) | | |
| 63 | N-[(3-Cyclopropylisoxazol-5- | 9.54 (t, J = 6.1, 1H), 8.78 (s, 1H), | 496.0 | SM27 |
| | yl)methyl]-6-methyl-5- | 7.98 (s, 1H), 7.95 (d, J = 7.8 Hz, 1H), | | |
| | (methylsulfonyl)-2-oxo-1-[3- | 7.86 (t, J = 7.8 Hz, 1H), 7.79 (d, | | |
| | (trifluoromethyl)phenyl]-1,2- | J = 7.8, 1H), 6.04 (s, 1H), 4.56 (d, | | |
| | dihydropyridine-3- | J = 6.2 Hz, 2H), 3.29 (s, 3H), 2.38 (s, | | |
| | carboxamide | 3H), 1.98-1.91 (m, 1H), | | |
| | | 0.99-0.93 (m, 2H), 0.73-0.68 (m, 2H) | | |
| 64 | 5-[(6-Cyanopyridin-3- | 9.25 (dd, J = 1.9, 0.4 Hz, 1H), | 477.1 | SM24 |
| | yl)sulfonyl]-N,6-dimethyl-2- | 8.97-8.88 (m, 2H), 8.64 (dd, J = 8.2, 2.4 Hz, | | |
| | oxo-1-[3- | 1H), 8.33 (dd, J = 8.3, 0.7 Hz, | | |
| | (trifluoromethyl)phenyl]-1,2- | 1H), 7.95-7.88 (m, 2H), 7.82 (t, J = 8.0 Hz, | | |
| | dihydropyridine-3- | 1H), 7.74 (d, J = 8.1 Hz, | | |
| | carboxamide | 1H), 2.81 (d, J = 4.8 Hz, 3H), | | |
| | | 2.21 (s, 3H) | | |

### EXAMPLE 65

### 6-Methyl-N-[4-(methylsulfonyl)benzyl]-2-oxo-1-[3-(trifluoromethyl)phenyl]-5-({4-[(trimethylsilyl)ethynl]phenyl}sulfinyl)-1,2-dihydropyridine-3-carboxamide

4-Bromobenzenethiol (177 mg, 1 mmol), tributylstannyl chloride (325 mg) and potassium carbonate (0.5 g) were mixed in acetonitrile and stirred overnight. The mixture was filtered and evaporated and the residue was dissolved in DMF (4 ml). 5-Iodo-6-methyl-N-[4-(methylsulfonyl)benzyl]-2-oxo-1-[3-(trifluoromethyl)phenyl]-1,2-dihydropyridine-3-carboxamide, starting material SM1 (590 mg) and bis(tri-t-butylphosphine)palladium (25 mg) were added. The mixture was degassed by bubbling argon through for 2 min, and was then heated in a microwave reactor at 150° C. for 15 min. The reaction mixture was partitioned between EtOAc and brine. The organic phase was filtered and evaporated to give a brown residue that was further purified by HPLC to afford the sulfide. The sulfide was dissolved in HOAc (2 ml). Hydrogen peroxide (0.5 ml of a 35% aq. solution) was added and the mixture heated to 50° C. for 30 min, whereupon the mixture was injected and purified on a HPLC, to yield 5-[(4-bromophenyl)sulfinyl]-6-methyl-N-[4-(methylsulfonyl)benzyl]-2-oxo-1-[3-(trifluoromethyl)phenyl]-1,2-dihydropyridine-3-carboxamide (60 mg).

5-[(4-Bromophenyl)sulfinyl]-6-methyl-N-[4-(methylsulfonyl)benzyl]-2-oxo-1-[3-(trifluoromethyl)phenyl]-1,2-dihydropyridine-3-carboxamide (180 mg) and DABCO (90 mg) were dissolved in DMF (10 ml). Ethynyl(trimethyl)silane (0.10 ml) was added followed by bis(tri-tert-butylphosphoranyl)palladium (40 mg). The reaction mixture was stirred for 72 h and then partitioned between EtOAc and brine. The organic phase was evaporated and the residue purified by HPLC to yield the title compound (50 mg).

¹H NMR (299.946 MHz, DMSO-d₆) δ 9.64 (t, J=6.0 Hz, 1H), 8.35 (d, J=2.0 Hz, 1H), 8.01 (d, J=5.9 Hz, 1H), 7.93 (t, J=3.5 Hz, 1H), 7.89-7.77 (m, 4H), 7.73-7.65 (m, 4H), 7.49 (d, J=8.3 Hz, 2H), 4.61-4.45 (m, 2H), 3.15 (s, 3H), 2.33 (s, 3H), 0.24 (s, 9H);

APCI-MS m/z: 685.3.

### EXAMPLE 66

### 5-[(4-Ethynylphenyl)sulfinyl]-6-methyl-N-[4-(methylsulfonyl)benzyl]-2-oxo-1-[3-(trifluoromethyl)phenyl]-1,2-dihydropyridine-3-carboxamide

5-[(4-Bromophenyl)sulfinyl]-6-methyl-N-[4-(methylsulfonyl)benzyl]-2-oxo-1-[3-(trifluoromethyl)phenyl]-1,2-dihydropyridine-3-carboxamide, intermediate prepared in Example 65, (1.68 g, 2.5 mmol) and DABCO (1.4 g, 12.5 mmol) were dissolved in DMF (10 ml). Ethynyl(trimethyl)silane (0.80 ml) was added followed by bis(tri-tert-butylphosphoranyl)palladium (100 mg, 0.2 mmol). The reaction mixture was stirred at 50° C. for 3 h and then poured onto crushed ice. The precipitate was collected and dried and further purified by column chromatography to provide 6-methyl-N-[4-(methylsulfonyl)benzyl]-2-oxo-1-[3-(trifluoromethyl)phenyl]-5-({4-[(trimethylsilyl)ethynyl]phenyl}sulfinyl)-1,2-dihydropyridine-3-carboxamide. This material was dissolved in MeOH (30 ml) and caesium fluoride (0.5 g) was added. After 10 min the reaction mixture was diluted with EtOAc (30 ml) and filtered through silica (20 g). The solvent was removed and the residue purified by HPLC to give the title compound (475 mg).

¹H NMR (399.99 MHz, DMSO-d₆) δ 9.65 (t, J=6.0 Hz, 1H), 8.38 (d, J=1.9 Hz, 1H), 8.01 (s, 1H), 7.93 (d, J=7.3 Hz, 1H), 7.89-7.77 (m, 4H), 7.74-7.68 (m, 4H), 7.50 (d, J=8.1 Hz, 2H), 4.61-4.46 (m, 2H), 4.42 (s, 1H), 3.15 (s, 3H), 2.33 (s, 3H);

APCI-MS m/z: 613.3 (MH⁺).

### EXAMPLE 67

### 6-Methyl-N-[4-(methylsulfonyl)benzyl]-2-oxo-5-{[4-(phenylethynyl)phenyl]sulfinyl}-1-[3-(trifluoromethyl)phenyl]-1,2-dihydropyridine-3-carboxamide

5-[(4-Ethynylphenyl)sulfinyl]-6-methyl-N-[4-(methylsulfonyl)benzyl]-2-oxo-1-[3-(trifluoromethyl)phenyl]-1,2-dihydropyridine-3-carboxamide (Example 66, 61 mg, 0.1 mmol), DABCO (100 mg) and iodobenzene (0.15 ml) were mixed in DMF (2 ml). Bis(tri-tert-butylphosphoranyl)palladium (25 mg) was added and the mixture stirred at 50° C. for 2 h, then filtered through silica (2 g). Evaporation of the solvents afforded an oily residue that was purified by HPLC to give the title compound (15 mg).

¹H NMR (399.99 MHz, DMSO-d₆) δ 9.66 (t, J=5.9 Hz, 11H), 8.39 (d, J=2.1 Hz, 1H), 8.03 (s, 1H), 7.94 (d, J=7.3 Hz, 1H), 7.89-7.73 (m, 7H), 7.63-7.55 (m, 3H), 7.53-7.41 (m, 5H), 4.54 (dd, J=14.1, 6.0 Hz, 2H), 3.15 (s, 3H), 2.35 (s, 3H);

APCI-MS m/z: 689.1 (MH⁺).

### EXAMPLE 68

### 6-Methyl-N-[4-(methylsulfonyl)benzyl]-2-oxo-5-[(4-prop-1-yn-1-ylphenyl)sulfinyl]-1-[3-(trifluoromethyl)phenyl]-1,2-dihydropyridine-3-carboxamide

The title compound was made by a procedure analogous to that described in Example 67.

¹H NMR (399.99 MHz, DMSO-d₆) δ 9.66 (t, J=5.9 Hz, 1H), 8.39 (d, J=2.3 Hz, 1H), 8.02 (s, 1H), 7.93 (d, J=7.1 Hz, 1H), 7.88-7.78 (m, 4H), 7.67 (d, J=8.3 Hz, 2H), 7.60 (d, J=8.1 Hz, 2H), 7.50 (d, J=8.1 Hz, 2H), 4.53 (td, J=14.9, 9.2 Hz, 2H), 3.15 (s, 3H), 2.32 (s, 3H), 2.07 (s, 3H);

APCI-MS m/z: 627.2 (MH⁺).

### EXAMPLE 69

### 5-[(5-Cyanopyridin-2-yl)sulfinyl]-N,6-dimethyl-2-oxo-1-[3-(trifluoromethyl)phenyl]-1,2-dihydropyridine-3-carboxamide

5-Iodo-N,6-dimethyl-2-oxo-1-[3-(trifluoromethyl)phenyl]-1,2-dihydropyridine-3-carboxamide (SM24, 8.0 g) in dry acetonitrile (150 ml) was degassed with argon. 2,4-Dimethoxybenzyl thiol (Synth. Commun. 1998, 28, 3219-3233) (5.0 g), (±)-trans-1,2-diaminocyclohexane (3.1 g) and copper(I) iodide (0.35 g) were added in succession and the mixture was heated to reflux overnight under argon. After cooling to RT, the mixture was filtered through celite, the filtrate was concentrated and the residue was dissolved in DCM (500 ml) and washed with brine. The organics were dried (Na₂SO₄), filtered and evaporated. The residue was purified by chromatography (SiO₂, DCM-heptane-ethyl acetate 1:2:1 to 1:1:2 gradient) to yield 5-[(2,4-dimethoxybenzyl)thio]-N,6-dimethyl-2-oxo-1-[3-(trifluoromethyl)phenyl]-1,2-dihydropyridine-3-carboxamide (8.13 g).

¹H NMR (400 MHz, DMSO-d₆) δ 9.15 (q, J=4.8 Hz, 1H), 8.40 (s, 1H), 7.89 (d, J=8.0 Hz, 1H), 7.81 (t, J=7.9 Hz, 1H), 7.71 (s, 1H), 7.58 (d, J=7.8 Hz, 1H), 6.86 (d, J=8.3 Hz, 1H), 6.51 (d, J=2.3 Hz, 1H), 6.46 (dd, J=8.2, 2.4 Hz, 1H), 3.86 (s, 2H), 3.73 (s, 3H), 3.73 (s, 3H), 2.79 (d, J=4.8 Hz, 3H), 1.78 (s, 3H).

APCI-MS m/z: 493.1 (MH⁺).

5-[(2,4-Dimethoxybenzyl)thio]-N,6-dimethyl-2-oxo-1-[3-(trifluoromethyl)phenyl]-1,2-dihydropyridine-3-carboxamide (1.0 g) was dissolved in dry 1,2-dichloroethane (40 ml), trifluoroacetic acid (3 ml) was added and the mixture was heated to reflux overnight. The mixture was evaporated and then repeatedly re-evaporated with ethyl acetate (3×50 ml) to yield 5-mercapto-N,6-dimethyl-2-oxo-1-[3-(trifluoromethyl)phenyl]-1,2-dihydropyridine-3-carboxamide as a white solid which was used in subsequent steps without further purification.

APCI-MS m/z: 343.1 (MH⁺).

5-Mercapto-N,6-dimethyl-2-oxo-1-[3-(trifluoromethyl)phenyl]-1,2-dihydropyridine-3-carboxamide (690 mg) and 6-chloronicotinonitrile (277 mg) were taken in dioxane (30 ml), Cs₂CO₃ (650 mg) was added and the resulting mixture was stirred under argon at 40° C. overnight. The reaction mixture was concentrated, dissolved in DCM (100 ml) and washed with brine. The organics were dried (Na₂SO₄), filtered and evaporated. The residue was purified by HPLC to yield 5-[(5-cyanopyridin-2-yl)thio]-N,6-dimethyl-2-oxo-1-[3-(trifluoromethyl)phenyl]-1,2-dihydropyridine-3-carboxamide (530 mg).

¹H NMR (400 MHz, DMSO-d₆) δ 9.14 (q, J=4.8 Hz, 1H), 8.87 (d, J=1.4 Hz, 1H), 8.37 (s, 1H), 8.17 (dd, J=8.5, 2.1 Hz, 1H), 7.99 (s, 1H), 7.93 (d, J=7.6 Hz, 1H), 7.85 (t, J=7.8 Hz, 1H), 7.79 (d, J=8.1 Hz, 1H), 7.49 (dd, J=8.5, 0.7 Hz, 1H), 2.79 (d, J=4.8 Hz, 3H), 2.15 (s, 3H).

APCI-MS m/z: 445.1 (MH⁺).

5-[(5-Cyanopyridin-2-yl)thio]-N,6-dimethyl-2-oxo-1-[3-(trifluoromethyl)phenyl]-1,2-dihydropyridine-3-carboxamide (500 mg) was taken in acetic acid (6 ml), hydrogen peroxide (33%, 1.5 ml) was added and the mixture was stirred at 50° C. After 2 h the reaction mixture was chromatographed (HPLC) to yield the title sulfoxide (375 mg).

¹H NMR (400 MHz, DMSO-d₆) δ 9.12 (ddd, J=7.1, 1.7, 0.4 Hz, 1H), 8.98 (d, J=2.7 Hz, 1H), 8.67 (dd, J=8.1, 1.6 Hz, 1H), 8.42 (d, J=6.9 Hz, 1H), 8.24 (dtd, J=8.2, 1.0, 0.1 Hz, 1H), 8.01-7.71 (m, 4H), 2.74 (d, J=4.8 Hz, 3H), 2.35 (d, J=1.1 Hz, 3H), APCI-MS m/z: 461.1 (MH⁺).

### EXAMPLES 70 TO 76

The following compounds were synthesised using an analogous method to that described for Example 69.

| Ex. | Compound | ¹H NMR | m/z |
|---|---|---|---|
| 70 | 6-({2-Methyl-5- | 9.07-8.96 (m, 2H), 8.53 (dd, J = 8.1, | 479.0 |
| | (methylcarbamoyl)-6-oxo-1- | 2.1 Hz 1H), 8.43 (d, J = 6.7 Hz, | |
| | [3-(trifluoromethyl)phenyl]- | 1H), 8.28 (s, 1H), 8.15 (d, J = 8.1 Hz, | |
| | 1,6-dihydropyridin-3- | 1H), 8.02-7.73 (m, 5H), | |
| | yl}sulfinyl)nicotinamide | 2.74 (d, J = 4.8 Hz, 3H), 2.36 (s, 3H) | |
| 71 | 5-[(5-Chloropyridin-2- | 9.00 (s, J = 2.1 Hz, 1H), 8.76 (dd, J = 4.8, | 499.9, |
| | yl)sulfinyl]-N,6-dimethyl-2- | 2.1 Hz, 1H), 8.44 (d, J = 8.1 Hz, | 471.9 |
| | oxo-1-[3-(trifluoromethyl)- | 1H), 8.30 (dd, J = 8.3, 1.8 Hz, | |
| | phenyl]-1,2-dihydropyridine- | 1H), 8.07 (dd, J = 8.4, 1.3 Hz, 1H), | |
| | 3-carboxamide | 8.01-7.72 (m, 4H), 2.75 (d, J = 4.8 Hz, | |
| | | 3H), 2.33 (d, J = 0.9 Hz, 3H) | |
| 72 | 5-[(5-Bromopyridin-2- | 9.00 (s, 1H), 8.82 (dd, J = 4.7, 2.0 Hz, | 515.9, |
| | yl)sulfinyl]-N,6-dimethyl-2- | 1H), 8.46-8.40 (m, 2H), | 513.9 |
| | oxo-1-[3-(trifluoromethyl)- | 8.03-7.72 (m, 5H), 2.75 (d, J = 4.8 Hz, | |
| | phenyl]-1,2-dihydropyridine- | 3H), 2.33 (s, 3H) | |
| | 3-carboxamide | | |
| 73 | 5-[(5-Cyanopyridin-2- | 9.64 (t, J = 6.1 Hz, 1H), 9.13 (dd, J = 7.6, | 615.0 |
| | yl)sulfinyl]-6-methyl-N-[4- | 1.4 Hz, 1H), 8.66 (dd, J = 8.2, | |
| | (methylsulfonyl)benzyl]-2- | 1.4 Hz, 1H), 8.45 (d, J = 7.6 Hz, | |
| | oxo-1-[3-(trifluoromethyl)- | 1H), 8.23 (d, J = 8.2 Hz, 1H), | |
| | phenyl]-1,2-dihydropyridine- | 8.03-7.72 (m, 6H), 7.50 (d, J = 8.3 Hz, | |
| | 3-carboxamide | 2H), 4.53 (m, 2H), 3.16 (s, 3H), | |
| | | 2.36 (d, J = 0.9 Hz, 3H) | |
| 74 | 5-[(5-Bromopyrimidin-2- | 9.21 (d, J = 4.5 Hz, 2H), 9.02 (q, J = 4.9 Hz, | 514.9, |
| | yl)sulfinyl]-N,6-dimethyl-2- | 1H), 8.69 (d, J = 10.1 Hz, | 516.9 |
| | oxo-1-[3-(trifluoromethyl)- | 1H), 8.00-7.66 (m, 4H), 2.77 (d, J = 4.8 Hz, | |
| | phenyl]-1,2-dihydropyridine- | 3H), 2.27 (d, J = 1.4 Hz, 3H) | |
| | 3-carboxamide | | |
| 75 | 5-[(6-Bromopyridazin-3- | 9.00 (q, J = 4.8 Hz, 1H), 8.51 (d, J = 6.9 Hz, | 515.2, |
| | yl)sulfinyl]-N,6-dimethyl-2- | 1H), 8.36 (d, J = 8.8 Hz, | 517.1 |
| | oxo-1-[3-(trifluoromethyl)- | 1H), 8.19 (dd, J = 8.9, 1.0 Hz, 1H), | |
| | phenyl]-1,2-dihydropyridine- | 8.00-7.71 (m, 4H), 2.76 (d, J = 4.8 Hz, | |
| | 3-carboxamide | 3H), 2.33 (d, J = 1.6 Hz, 3H) | |

### EXAMPLE 76

### 5-[(6-Cyanopyridin-3-yl)sulfinyl]-N,6-dimethyl-2-oxo-1-[3-(trifluoromethyl)phenyl]-1,2-dihydropyridine-3-carboxamide

A mixture of 5-mercapto-N,6-dimethyl-2-oxo-1-[3-(trifluoromethyl)phenyl]-1,2-dihydropyridine-3-carboxamide, intermediate described in Example 69, (690 mg), 5-chloropyridine-2-carbonitrile (277 mg) and (±)-trans-1,2-diaminocyclohexane (118 mg) in dry acetonitrile (10 ml) was degassed with argon. Copper (I) iodide (95 mg) was added and the mixture was stirred overnight at 82° C. The mixture was cooled to RT, filtered through celite, the filtrate was concentrated, the residue was dissolved in DCM (100 ml) and washed with brine. The organics were dried (Na₂SO₄), filtered and evaporated. The residue was purified by HPLC to yield 5-[(6-cyanopyridin-3-yl)thio]-N,6-dimethyl-2-oxo-1-[3-(trifluoromethyl)phenyl]-1,2-dihydropyridine-3-carboxamide (328 mg). APCI-MS m/z: 445.1 (MH⁺).

To 5-[(6-cyanopyridin-3-yl)thio]-N,6-dimethyl-2-oxo-1-[3-(trifluoromethyl)phenyl]-1,2-dihydropyridine-3-carboxamide (325 mg) in acetic acid (6 ml) was added hydrogen peroxide (33%, 1.5 ml) and the resulting mixture was stirred at 50° C. After 90 min the reaction mixture was chromatographed (HPLC) to yield title compound (218 mg).

¹H NMR (400 MHz, DMSO-d₆) δ 8.97 (s, 2H), 8.40 (ddd, J=7.8, 2.0, 0.4 Hz, 1H), 8.34-8.28 (m, 2H), 8.01-7.78 (m, 4H), 2.74 (dd, J=4.8, 1.1 Hz, 3H), 2.34 (s, 3H);

APCI-MS m/z: 461.1 (MH⁺).

### EXAMPLES 77 TO 78

The following compounds were synthesised using an analogous method to that described for Example 76.

| Ex. | Compound | ¹H NMR | m/z |
|---|---|---|---|
| 77 | 5-[(5-Cyano-2-thienyl)- | 9.03 (q, J = 4.8 Hz, 1H), 8.65 (d, J = 0.9 Hz, | 466.3 |
| | sulfinyl]-N,6-dimethyl-2-oxo- | 1H), 8.09 (d, J = 4.1 Hz, | |
| | 1-[3-(trifluoromethyl)- | 1H), 8.01-7.70 (m, 5H), 2.79 (d, J = 4.6 Hz, | |
| | phenyl]-1,2-dihydropyridine- | 3H), 2.22 (s, 3H) | |
| | 3-carboxamide | | |
| 78 | 5-(1H-Imidazol-2-ylsulfinyl)- | 9.72 (t, J = 6.1 Hz, 1H), 8.83 (d, J = 8.1 Hz, | 579.0 |
| | 6-methyl-N-[4- | 1H), 8.01-7.70 (m, 7H), | |
| | (methylsulfonyl)benzyl]-2- | 7.54 (d, J = 8.3 Hz, 2H), 7.31 (s, | |
| | oxo-1-[3-(trifluoromethyl)- | 2H), 4.59 (m, 2H), 3.17 (s, 3H), | |
| | phenyl]-1,2-dihydropyridine- | 2.14 (d, J = 6.8 Hz, 3H) | |
| | 3-carboxamide | | |

### EXAMPLE 79

### 6-Methyl-5-[(methylamino)sulfonyl]-N-[4-(methylsulfonyl)benzyl]-2-oxo-1-[3-(trifluoromethyl)phenyl]-1,2-dihydro-pyridine-3-carboxamide

Benzyl thiol (124 mg) and tributylstannyl chloride (325 mg) were mixed in acetonitrile (50 ml) and stirred overnight, whereupon the mixture was filtered through a short column of silica which was washed with DCM. Evaporation of the solvents afforded an oily residue which was dissolved in DMF (4 ml). 5-Iodo-6-methyl-N-[4-(methylsulfonyl)benzyl]-2-oxo-1-[3-(trifluoromethyl)phenyl]-1,2-dihydropyridine-3-carboxamide starting material SM 1 (590 mg) was added, followed by bis(tri-t-butylphosphine)palladium (100 mg). The resulting mixture was degassed by passing argon through the solution (5 min) and then heated in a microwave reactor to 150° C. for 15 min. The reaction mixture was partitioned between EtOAc and brine. The organic phase was filtered and evaporated to yield a solid residue which was purified by recrystallisation from 2-propanol. The resulting crystalline material was dissolved in HOAc (50 ml). Water (5 ml) was added and chlorine gas bubbled through the solution for 1 min. To remove excess chlorine, argon was bubbled through for another 15 min and the reaction mixture was freeze dried to yield 2-methyl-5-({[4-(methylsulfonyl)benzyl]amino}carbonyl)-6-oxo-1-[3-(trifluoromethyl)phenyl]-1,6-dihydropyridine-3-sulfonyl chloride, which was used in subsequent steps without further purification.

2-Methyl-5-({[4-(methylsulfonyl)benzyl]amino}carbonyl)-6-oxo-1-[3-(trifluoromethyl)phenyl]-1,6-dihydropyridine-3-sulfonyl chloride (40 mg) was dissolved in a 2M solution of methylamine in THF (1 ml). After 10 min the mixture was evaporated to dryness and the residue purified by HPLC to afford the title compound (42 mg).

¹H NMR (399.99 MHz, DMSO-d₆) δ 9.70 (t, J=6.1 Hz, 1H), 8.75 (s, 1H), 8.03 (s, 1H), 7.95-7.75 (m, 6H), 7.54 (d, J=8.4 Hz, 2H), 4.59 (d, J=6.1 Hz, 2H), 3.17 (s, 3H), 2.52 (d, J=5.0 Hz, 3H), 2.30 (s, 3H);

APCI-MS m/z: 558.1 (MH⁺).

### EXAMPLES 80 TO 85

The following compounds were synthesised using an analogous method to that described for Example 79.

| Ex. | Compound | ¹H NMR | m/z |
|---|---|---|---|
| 80 | 5-(Anilinosulfonyl)-6- | 10.51 (s, 1H), 9.62 (t, J = 6.0 Hz, 1H), | 620.1 |
| | methyl-N-[4- | 8.76 (s, 1H), 7.98-7.77 (m, 5H), | |
| | (methylsulfonyl)benzyl]-2- | 7.72 (d, J = 7.6 Hz, 1H), 7.53 (d, J = 8.1 Hz, | |
| | oxo-1-[3-(trifluoromethyl)- | 2H), 7.32 (t, J = 8.0 Hz, 2H), | |
| | phenyl]-1,2-dihydropyridine- | 7.16-7.10 (m, 3H), 4.56 (d, J = 6.0 Hz, | |
| | 3-carboxamide | 2H), 3.17 (s, 3H), 2.18 (s, 3H) | |
| 81 | 6-Methyl-N-[4- | 9.76 (s, 1H), 9.70 (t, J = 5.6 Hz, 1H), | 657.1 |
| | (methylsulfonyl)benzyl]-5- | 8.77 (s, 1H), 8.26 (s, 1H), | |
| | {[(2-morpholin-4- | 8.03-7.75 (m, 5H), 7.54 (d, J = 8.0 Hz, 2H), | |
| | ylethyl)amino]sulfonyl}-2- | 4.58 (d, J = 6.0 Hz, 2H), 4.07-3.89 (m, | |
| | oxo-1-[3-(trifluoromethyl)- | 2H), 3.74-3.58 (m, 2H), | |
| | phenyl]-1,2-dihydropyridine- | 3.57-3.02 (m, 11H), 2.32 (s, 3H) | |
| | 3-carboxamide | | |
| 82 | 5-{[(2-Cyanoethyl)(methyl)- | 9.65 (s, 1H), 8.72 (s, 1H), | 611.1 |
| | amino]sulfonyl}-6-methyl-N- | 7.98-7.73 (m, 6H), 7.54 (d, J = 7.3 Hz, 2H), | |
| | [4-(methylsulfonyl)benzyl]-2- | 4.58 (d, J = 4.1 Hz, 2H), 3.40-3.34 (m, | |
| | oxo-1-[3-(trifluoromethyl)- | 2H), 3.17 (s, 3H), 2.88-2.80 (m, 5H), | |
| | phenyl]-1,2-dihydropyridine- | 2.30 (s, 3H) | |
| | 3-carboxamide | | |
| 83 | 6-Methyl-N-[4- | 10.05 (s, 1H), 9.64 (s, 1H), 8.63 (s, | 706.1 |
| | (methylsulfonyl)benzyl]-5- | 1H), 7.99 (s, 1H), 7.95-7.74 (m, 6H), | |
| | {[(6-morpholin-4-ylpyridin- | 7.52 (d, J = 8.4 Hz, 2H), 7.38 (dd, J = 9.0, | |
| | 3-yl)amino]sulfonyl}-2-oxo- | 2.8 Hz, 1H), 6.82 (d, J = 9.2 Hz, | |
| | 1-[3-(trifluoromethyl)- | 1H), 4.56 (d, J = 5.9 Hz, 2H), 3.66 (t, | |
| | phenyl]-1,2-dihydropyridine- | J = 5.0 Hz, 4H), 3.51-3.26 (m, 4H), | |
| | 3-carboxamide | 3.17 (s, 3H), 2.16 (s, 3H) | |
| 84 | 6-Methyl-N-[4- | 9.68 (t, J = 6.1 Hz, 1H), 8.66 (s, 1H), | 614.1 |
| | (methylsulfonyl)benzyl]-5- | 8.03 (s, 1H), 7.98-7.80 (m, 5H), | |
| | (morpholin-4-ylsulfonyl)-2- | 7.54 (d, J = 8.4 Hz, 2H), 4.58 (d, J = 6.1 Hz, | |
| | oxo-1-[3-(trifluoromethyl)- | 2H), 3.67 (t, J = 4.5 Hz, 4H), | |
| | phenyl]-1,2-dihydropyridine- | 3.17 (s, 3H), 3.09 (dd, J = 14.1, 11.0 Hz, | |
| | 3-carboxamide | 4H), 2.33 (s, 3H) | |
| 85 | 6-Methyl-N-[4- | 10.88 (s, 1H), 9.62 (t, J = 6.3 Hz, 1H), | 621.1 |
| | (methylsulfonyl)benzyl]-2- | 8.73 (s, 1H), 8.36 (td, J = 4.7, 1.7 Hz, | |
| | oxo-5-[(pyridin-3- | 1H), 7.99 (s, 1H), 7.95-7.73 (m, 6H), | |
| | ylamino)sulfonyl]-1-[3- | 7.61-7.48 (m, 3H), 7.40 (dd, J = 13.0, | |
| | (trifluoromethyl)phenyl]-1,2- | 0.7 Hz, 1H), 4.56 (d, J = 6.1 Hz, | |
| | dihydropyridine-3- | 2H), 3.17 (s, 3H), 2.28 (d, J = 6.4 Hz, | |
| | carboxamide | 3H) | |

### EXAMPLE 86

### 2-Methyl-5-({[4-(methylsulfonyl)benzyl]amino}carbonyl)-6-oxo-1-[3-(trifluoromethyl)phenyl]-1,6-dihydropyridine-3-sulfonic acid

2-Methyl-5-({[4-(methylsulfonyl)benzyl]amino}carbonyl)-6-oxo-1-[3-(trifluoromethyl)phenyl]-1,6-dihydropyridine-3-sulfonyl chloride, intermediate described in Example 79, (60 mg) was dissolved in THF (5 ml). Imidazole (100 mg) was added and after 10 min the mixture was evaporated to dryness and the residue purified by HPLC to afford the title compound (22 mg).

¹H NMR (299.946 MHz, DMSO-d₆) δ 9.84 (t, J=6.1 Hz, 1H), 8.85 (s, 1H), 7.96-7.67 (m, 6H), 7.54 (d, J=8.4 Hz, 2H), 4.58 (d, J=6.1 Hz, 2H), 3.16 (s, 3H), 2.28 (d, J=6.2 Hz, 3H);

APCI-MS m/z: 565.1 (MH⁺).

### Preparation of Starting Materials

The starting materials for the Examples 1 to 86 are either commercially available or are readily prepared by standard methods from known materials. For example, the following reactions are illustrations, but not a limitation, of the preparation of some of the starting materials.

### Starting Material SM1

### 5-Iodo-6-methyl-N-[4-(methylsulfonyl)benzyl]-2-oxo-1-[3-(trifluoromethyl)phenyl]-1,2-dihydropyridine-3-carboxamide

To an ice-cooled solution of 3-(trifluoromethyl)aniline (64.5 g, 0.40 mol) and triethylamine (60 ml) in acetone (700 ml) was added dropwise, ethyl 3-chloro-3-oxopropanoate (63.6 g, 0.42 mol) in acetone (50 ml). After the addition (approx. 30 minutes) stirring was continued at RT overnight. The solvents were removed and water (1200 ml) was added. The resulting precipitate was filtered off, thoroughly washed twice with water and then dried to afford ethyl 3-oxo-3-{[3-(trifluoromethyl)phenyl]amino}propanoate as yellow powder (109 g, 99%).

¹H NMR (399.99 MHz, CDCl₃): δ 9.52 (1H, s); 7.87 (1H, s); 7.78 (1H, d); 7.46 (1H, t); 7.39 (1H, d); 4.29 (2H, q); 3.50 (2H, s); 1.35 (3H, t);

APCI-MS m/z: 276.1 [MH].

To a solution of ethyl 3-oxo-3-{[3-(trifluoromethyl)phenyl]amino}propanoate (19.2 g, 70 mmol) and sodium methoxide (7.6 g, 140 mmol) in EtOH (250 ml) was added 4-methoxybut-3-en-2-one (90%) (7.72 g, 77 mmol). After the addition, the reaction mixture was refluxed for 2 h and then cooled. Water (50 ml) and 2M NaOH were added and the mixture was stirred at RT overnight. The organic solvents were removed and the reaction mixture was extracted (washed) with EtOAc. The water phases were acidified with hydrochloric acid to pH 3-4, an orange coloured precipitate appeared and was filtered off, washed with water and dried. Recrystallisation twice from heptane/EtOAc (4:1) afforded 6-methyl-2-oxo-1-[3-(trifluoromethyl)phenyl]-1,2-dihydropyridine-3-carboxylic acid (12 g, 58%) as a white powder.

¹H NMR (399.99 MHz, CDCl₃): δ 13.68 (1H, s); 8.54 (1H, d); 7.86 (1H, d); 7.79 (1H, t); 7.55 (1H, brs); 7.48 (1H, d); 6.58 (1H, d); 2.16 (3H, s);

APCI-MS m/z: 298.1 [MH].

A mixture of 6-methyl-2-oxo-1-[3-(trifluoromethyl)phenyl]-1,2-dihydropyridine-3-carboxylic acid (7.43 g, 25 mmol), HATU (10.5 g, 27.5 mmol), HOAT (3.75 g, 27.5 mmol) and DIPEA (14.2 ml, 82.5 mmol) in NMP (65 ml) was reacted for 1 h, then 4-methylsulphonylbenzyl amine hydrochloride (5.8 g, 26 mmol) was added. After 1 h, the reaction mixture was slowly poured into stirred ice water (1 L). A powder was formed, and the water mixture was acidified to pH 3 with citric acid (0.5 M), and stirring was continued for 1 h. The precipitate was filtered off, washed with water and dried in vacuum overnight. Recrystallisation from EtQAc gave 8.1 g (70%) of 6-methyl-N-[4-(methylsulfonyl)benzyl]-2-oxo-1-[3-(trifluoromethyl)phenyl]-1,2-dihydropyridine-3-carboxamide.

¹H NMR (399.99 MHz, CDCl₃): δ 10.00 (1H, brt); 8.60 (1H, d); 7.88 (2H, d); 7.83 (1H, d); 7.76 (1H, t); 7.53 (3H, m); 7.46 (1H, d); 6.49 (1H, d); 4.68 (2H, m); 3.03 (3H, s); 2.10 (3H, s);

APCI-MS m/z: 465.1 [MH⁺].

To a solution of 6-methyl-N-[4-(methylsulfonyl)benzyl]-2-oxo-1-[3-(trifluoromethyl)phenyl]-1,2-dihydropyridine-3-carboxamide (200 mg, 0.43 mmol) in MeCN (1.5 ml) at RT and under argon, was added trifluoromethanesulfonic acid (1 ml) followed by N-iodosuccinimide (97 mg, 0.43 mmol). After 45 min, the reaction mixture was diluted with DCM, washed with aqueous NaHCO₃, with aqueous NaS₂O₄ and water, dried (Na₂SO₄), and evaporated to give the title compound SM1 (200 mg).

¹H NMR (399.99 MHz, CDCl₃): δ 9.85 (1H, brt); 8.90 (1H, d); 7.88 (2H, d); 7.76 (2H, m); 7.50 (2H, d); 7.48 (1H, s); 7.40 (1H, d); 4.65 (2H, m); 3.03 (3H, s); 2.32 (3H, s);

APCI-MS m/z: 591.0 [MH⁺].

Starting material SM1 was used in the synthesis of the compounds of Examples: 3, 4, 12, 13, 31, 32, 33, 34, 35, 36, 37, 59, 60, 65 and 79.

### Starting Materials SM2 to SM 27

The following compounds were synthesised using analogous methods to those described for SM1.

| | Compound | ¹H NMR | m/z | SM in |
|---|---|---|---|---|
| SM2 | 5-Iodo-2-oxo-N-[3-(2- | 9.43 (t, J = 5.9 Hz, 1H), 8.46 (d, J = 2.6 Hz, | 534.0 | Ex. |
| | oxopyrrolidin-1-yl)propyl]-1- | 1H), 8.38 (t, J = 2.7 Hz, | | 2, 57 |
| | [3-(trifluoromethyl)phenyl]- | 1H), 7.98 (d, J = 1.8 Hz, 1H), | | |
| | 1,2-dihydropyridine-3- | 7.91-7.73 (m, 3H), 3.35-3.13 (m, 6H), | | |
| | carboxamide | 2.17 (t, J = 8.2 Hz, 2H), | | |
| | | 1.88 (quintet, J = 7.5 Hz, 2H), | | |
| | | 1.66 (quintet, J = 6.9 Hz, 2H). | | |
| SM3 | N-Cyclopropyl-5-iodo-6- | Used directly in next step | | 1, 5, 8, 9, |
| | methyl-2-oxo-1-[3- | | | 24 |
| | (trifluoromethyl)phenyl]-1,2- | | | |
| | dihydropyridine-3- | | | |
| | carboxamide | | | |
| SM4 | N-{[5-(Cyclopropylsulfonyl)- | 10.20 (brs, 1H), 9.04 (d, J = 2.1 Hz, | 604.2 | 6 |
| | pyridin-2-yl]methyl}-5-iodo- | 1H), 8.80 (d, J = 2.7 Hz, 1H), | | |
| | 2-oxo-1-[3-(trifluoromethyl)- | 8.15-8.10 (m, 1H), 7.82 (d, J = 2.7 Hz, | | |
| | phenyl]-1,2-dihydropyridine- | 1H), 7.79 (s, 1H), 7.73 (d, J = 8.2 Hz, | | |
| | 3-carboxamide | 1H), 7.69 (d, J = 5.3, 1H), | | |
| | | 7.61 (d, J = 8.2 Hz, 1H), 7.50 (d, J = 8.2 Hz, | | |
| | | 1H), 4.86 (d, J = 5.7 Hz, 2H), | | |
| | | 2.50-2.42 (m, 1H), 1.42-1.34 (m, | | |
| | | 2H), 1.12-1.03 (m, 2H) | | |
| SM5 | 5-Iodo-6-methyl-N-{[5- | 10.00 (t, J = 5.60 Hz, 1H), 8.98 (d, | | 7, 28, 38, |
| | (methylsulfonyl)pyridin-2- | J = 1.84 Hz, 1H), 8.59 (s, 1H), | | 56 |
| | yl]methyl}-2-oxo-1-[3- | 8.26 (dd, J = 8.25, 2.39 Hz, 1H), | | |
| | (trifluoromethyl)phenyl]-1,2- | 7.94-7.72 (m, 4H), 7.56 (d, J = 8.25 Hz, | | |
| | dihydropyridine-3- | 1H), 4.71 (d, J = 5.69 Hz, 2H), | | |
| | carboxamide | 3.28 (s, 3H), 2.21 (s, 3H) | | |
| SM6 | N-[(2S)-2-Hydroxypropyl]-5- | Used directly in next step | | 10 |
| | iodo-6-methyl-2-oxo-1-[3- | | | |
| | (trifluoromethyl)phenyl]-1,2- | | | |
| | dihydropyridine-3- | | | |
| | carboxamide | | | |
| SM7 | 5-Iodo-6-methyl-2-oxo-1-[3- | Used directly in next step | | 11, 58 |
| | (trifluoromethyl)phenyl]-1,2- | | | |
| | dihydropyridine-3- | | | |
| | carboxamide | | | |
| SM8 | N-Cyclopropyl-1-(3,5- | | 431.0 | 14 |
| | difluorophenyl)-5-iodo-6- | | | |
| | methyl-2-oxo-1,2-dihydro- | | | |
| | pyridine-3-carboxamide | | | |
| SM9 | N-{[5-(Ethylsulfonyl)pyridin- | 10.01 (t, J = 5.7 Hz, 1H), 8.93 (dd, | 605.9 | 15 |
| | 2-yl]methyl}-5-iodo-6- | J = 2.3, 0.6 Hz, 1H), 8.59 (s, 1H), | | |
| | methyl-2-oxo-1-[3- | 8.23 (dd, J = 8.3, 2.4 Hz, 1H), | | |
| | (trifluoromethyl)phenyl]-1,2- | 7.95-7.72 (m, 4H), 7.57 (d, J = 16.8 Hz, | | |
| | dihydropyridine-3- | 1H), 4.72 (d, J = 5.7 Hz, 2H), | | |
| | carboxamide | 3.37 (q, J = 9.2 Hz, 2H), 2.21 (s, | | |
| | | 3H), 1.11 (t, J = 7.4 Hz, 3H) | | |
| SM10 | 1-(3,5-Difluorophenyl)-N- | Used directly in next step | | 16 |
| | {[5-(ethylsulfonyl)pyridin-2- | | | |
| | yl]methyl}-5-iodo-6-methyl- | | | |
| | 2-oxo-1,2-dihydropyridine-3- | | | |
| | carboxamide | | | |
| SM11 | 1-(3,5-Dichlorophenyl)-N- | Used directly in next step | | 17 |
| | {[5-(ethylsulfonyl)pyridin-2- | | | |
| | yl]methyl}-5-iodo-6-methyl- | | | |
| | 2-oxo-1,2-dihydropyridine-3- | | | |
| | carboxamide | | | |
| SM12 | 5-Iodo-N-methyl-2-oxo-1-[3- | | 423.1 | 18 |
| | (trifluoromethyl)phenyl]-1,2- | | | |
| | dihydropyridine-3- | | | |
| | carboxamide | | | |
| SM13 | 1-(3,5-Dichlorophenyl)-5- | Used directly in next step | | 19 |
| | iodo-N,6-dimethyl-2-oxo-1,2- | | | |
| | dihydropyridine-3- | | | |
| | carboxamide | | | |
| SM14 | 1-(3,5-Difluorophenyl)-N-[2- | Used directly in next step | | 20 |
| | (1H-imidazol-4-yl)ethyl]-5- | | | |
| | iodo-6-methyl-2-oxo-1,2- | | | |
| | dihydropyridine-3- | | | |
| | carboxamide | | | |
| SM15 | 1-(3,5-Difluorophenyl)-5- | Used directly in next step | | 21 |
| | iodo-6-methyl-N-(2- | | | |
| | morpholin-4-ylethyl)-2-oxo- | | | |
| | 1,2-dihydropyridine-3- | | | |
| | carboxamide | | | |
| SM16 | 1-(3,5-Difluorophenyl)-5- | Used directly in next step | | 22 |
| | iodo-N,6-dimethyl-2-oxo-1,2- | | | |
| | dihydropyridine-3- | | | |
| | carboxamide | | | |
| SM17 | 5-Iodo-6-methyl-N-[(3- | 9.75 (t, J = 5.9 Hz, 1H), 8.58 (s, 1H), | 518.0 | 23 |
| | methylisoxazol-5-yl)methyl]- | 7.93-7.87 (m, 2H), 7.82 (t, J = 7.8 Hz, | | |
| | 2-oxo-1-[3-(trifluoromethyl)- | 1H), 7.73 (d, J = 7.8 Hz, 1H), | | |
| | phenyl]-1,2-dihydropyridine- | 6.15 (s, 1H), 4.57 (d, J = 6.0 Hz, | | |
| | 3-carboxamide | 2H), 2.21 (s, 3H), 2.17 (s, 3H) | | |
| SM18 | N-[3-(1H-Imidazol-1- | Used directly in next step | | 25 |
| | yl)propyl]-5-iodo-6-methyl- | | | |
| | 2-oxo-1-[3-(trifluoromethyl)- | | | |
| | phenyl]-1,2-dihydropyridine- | | | |
| | 3-carboxamide | | | |
| SM19 | 5-Iodo-6-methyl-2-oxo-N-[3- | Used directly in next step | | 26 |
| | (1H-1,2,3-triazol-1-yl)- | | | |
| | propyl]-1-[3- | | | |
| | (trifluoromethyl)phenyl]-1,2- | | | |
| | dihydropyridine-3- | | | |
| | carboxamide | | | |
| SM20 | N-[(1-Hydroxycyclopropyl)- | Used directly in next step | | 27 |
| | methyl]-5-iodo-6-methyl-2- | | | |
| | oxo-1-[3-(trifluoromethyl)- | | | |
| | phenyl]-1,2-dihydropyridine- | | | |
| | 3-carboxamide | | | |
| SM21 | 5-Iodo-N-(2-methoxyethyl)- | Used directly in next step | | 29 |
| | 6-methyl-2-oxo-1-[3- | | | |
| | (trifluoromethyl)phenyl]-1,2- | | | |
| | dihydropyridine-3- | | | |
| | carboxamide | | | |
| SM22 | N-(2-Hydroxy-2- | Used directly in next step | | 30, 62 |
| | methylpropyl)-5-iodo-6- | | | |
| | methyl-2-oxo-1-[3- | | | |
| | (trifluoromethyl)phenyl]-1,2- | | | |
| | dihydropyridine-3- | | | |
| | carboxamide | | | |
| SM23 | N-(2-Hydroxy-2- | Used directly in next step | | 39, 41 |
| | methylpropyl)-5-iodo-6- | | | |
| | methyl-2-oxo-1-[3- | | | |
| | (trifluoromethyl)phenyl]-1,2- | | | |
| | dihydropyridine-3- | | | |
| | carboxamide | | | |
| SM24 | 5-Iodo-N,6-dimethyl-2-oxo- | Used directly in next step | | 40, 42, |
| | 1-[3-(trifluoromethyl)- | | | 43, 61, |
| | phenyl]-1,2-dihydropyridine- | | | 64, 69 |
| | 3-carboxamide | | | |
| SM25 | 5-[(4-Cyanophenyl)sulfinyl]- | | 447.0 | 44, 45, |
| | 6-methyl-2-oxo-1-[3- | | | 47, 48, |
| | (trifluoromethyl)phenyl]-1,2- | | | 49, 50, |
| | dihydropyridine-3-carboxylic | | | 51, 52, |
| | acid | | | 53, 54, |
| | | | | 55, 56 |
| SM26 | 5-[(4-Cyanophenyl)sulfinyl]- | Used directly in next step | | 46 |
| | 2-oxo-1-[3-(trifluoromethyl)- | | | |
| | phenyl]-1,2-dihydropyridine- | | | |
| | 3-carboxylic acid | | | |
| SM27 | 5-Iodo-6-methyl-N-[(3- | 9.73 (t, J = 5.9 Hz, 1H), 8.58 (s, 1H), | 543.9 | 63 |
| | cyclopropylisoxazol-5- | 7.93-7.87 (m, 2H), 7.82 (t, J = 7.8 Hz, | | |
| | yl)methyl]-2-oxo-1-[3- | 1H), 7.72 (d, J = 7.8 Hz, 1H), | | |
| | (trifluoromethyl)phenyl]-1,2- | 6.04 (s, 1H), 4.54 (d, J = 6.0 Hz, | | |
| | dihydropyridine-3- | 2H), 2.21 (s, 3H), 1.98-1.90 (m, | | |
| | carboxamide | 1H), 0.99-0.92 (m, 2H), | | |
| | | 0.76-0.67 (m, 2H) | | |

### SM28

### N-[4-(Cyclopropylsulfonyl)benzyl]-5-iodo-6-methyl-2-oxo-1-[3-(trifluoromethyl)phenyl]-1,2-dihydropyridine-3-carboxamide

The title compound was prepared using a procedure analogous to that described for SM1.

¹H NMR (CDCl₃): δ 9.86 (1H, t, J=5.8 Hz); 8.90 (1H, s); 7.83-7.80 (3H, m); 7.75 (1H, t, J=7.8 Hz); 7.49-7.47 (3H, m); 7.40 (1H, d, J=7.8 Hz); 4.66 (2H, t, J=5.7 Hz); 2.42 (1H, m); 2.31 (3H, s); 1.32 (2H, m); 1.01 (2H, m);

APCI-MS m/z: 617 [MH⁺].

### EXAMPLE 87

### 6-Methyl-N-[4-(methylsulfonyl)benzyl]-2-oxo-5-(phenylthio)-1-[3-(trifluoromethyl)phenyl]-1,2-dihydropyridine-3-carboxamide

Tributyl(phenylthio)stannane (400 mg, 1 mmol) and 5-iodo-6-methyl-N-[4-(methylsulfonyl)benzyl]-2-oxo-1-[3-(trifluoromethyl)phenyl]-1,2-dihydropyridine-3-carboxamide (SM1, 590 mg, 1 mmol) were dissolved in DMF (3 ml). Bis(tri-t-butylphosphine)palladium (50 mg, 0.1 mmol) was added and the mixture degassed by bubbling argon through the solution, whereupon it was heated in a microwave oven to 150° C. for 45 minutes. The reaction mixture was filtered and then directly applied to preparative HPLC. The appropriate fractions were pooled and freeze-dried to provide the title compound as a white solid (480 mg).

¹H-NMR (DMSO-d₆): δ 9.81 (t, J=6.1 Hz, 1H), 8.36 (s, 1H), 8.04 (s, 1H), 7.97-7.76 (m, 5H), 7.53 (d, J=8.4 Hz, 2H), 7.40-7.17 (m, 5H), 4.57 (d, J=6.2 Hz, 2H), 3.17 (s, 3H), 2.20 (s, 3H);

APCI-MS m/z: 572.9 [MH⁺].

### EXAMPLE 88

### 6-Methyl-N-[4-(methylsulfonyl)benzyl]-2-oxo-5-(phenylsulfinyl)-1-[3-(trifluoromethyl)phenyl]-1,2-dihydropyridine-3-carboxamide

6-Methyl-N-[4-(methylsulfonyl)benzyl]-2-oxo-5-(phenylthio)-1-[3-(trifluoromethyl)phenyl]-1,2-dihydropyridine-3-carboxamide (Example 87, 60 mg, 0.1 mmol) and sodium periodate (35 mg, 0.15 mmol) were mixed in methanol (10 ml) and water (2 ml) was added. The mixture was stirred at 60° C. overnight. A second portion of sodium periodate (50 mg) was added and the mixture was stirred for 4 h at 60° C. whereupon it was cooled, filtered through a short column of silica and evaporated. The pale yellow oily residue was subjected to preparative HPLC. The appropriate fractions were pooled and freeze-dried to provide the title compound as a white solid (9 mg).

¹H-NMR (DMSO-d₆): δ 9.66 (t, J=5.9 Hz, 1H), 8.41 (d, J=2.8 Hz, 1H), 8.07-7.43 (m, 13H), 4.63-4.44 (m, 2H), 3.15 (s, 3H), 2.34 (s, 3H);

APCI-MS m/z: 589.0 [MH⁺].

### EXAMPLE 89

### 6-Methyl-N-[4-(methylsulfonyl)benzyl]-2-oxo-5-(phenylylsulfonyl)-1-[3-(trifluoromethyl)phenyl]-1,2-dihydropyridine-3-carboxamide

6-Methyl-N-[4-(methylsulfonyl)benzyl]-2-oxo-5-(phenylthio)-1-[3-(trifluoromethyl)phenyl]-1,2-dihydropyridine-3-carboxamide (Example 87, 70 mg, 0.12 mmol) was dissolved in acetic acid (5 ml). Hydrogen peroxide (3 ml of 35% solution in water) was added and the mixture was stirred at 60° C. overnight. The reaction mixture was directly purified using semi-preparative HPLC. The appropriate fractions were pooled and freeze-dried to provide the title compound as a white solid (74 mg).

¹H-NMR (DMSO-d₆): δ 9.62 (t, J=6.1 Hz, 1H), 8.92 (s, 1H), 8.01-7.51 (m, 13H), 4.59 (d, J=6.1 Hz, 2H), 3.17 (s, 3H), 2.22 (s, 3H);

APCI-MS m/z: 604.9 [MH⁺].

### EXAMPLE 90

### 6-Methyl-5-(methylsulfinyl)-N-[4-(methylsulfonyl)benzyl]-2-oxo-1-[3-(trifluoromethyl)phenyl]-1,2-dihydropyridine-3-carboxamide

Tributylstannyl chloride (334 mg, 1 mmol) and sodium methylthiolate (70 mg, 1 mmol) were mixed and stirred in acetonitrile (20 ml) overnight. The reaction mixture was filtered through a short column of silica. The filtrate was evaporated and the residue dissolved in DMF (3 ml). 5-Iodo-6-methyl-N-[4-(methylsulfonyl)benzyl]-2-oxo-1-[3-(trifluoromethyl)phenyl]-1,2-dihydropyridine-3-carboxamide (SM1, 590 mg, 1 mmol) and bis(tri-t-butylphosphine)palladium (50 mg, 0.1 μmol) were added and the mixture degassed by bubbling argon through the solution, whereupon it was heated in a microwave oven to 150° C. for 45 minutes. The reaction mixture was filtered and then directly applied to preparative HPLC. The appropriate fractions were pooled and evaporated. This afforded 6-methyl-N-[4-(methylsulfonyl)benzyl]-5-(methylthio)-2-oxo-1-[3-(trifluoromethyl)phenyl]-1,2-dihydropyridine-3-carboxamide as an off-white solid (377 mg). This material was pure enough for subsequent elaborations. The methylthio compound (51 mg, 0.1 mmol) and sodium periodate (70 mg, 0.3 mmol) were mixed in methanol (10 ml) and water (2 ml) was added. The mixture was stirred at 60° C. overnight, whereupon it was cooled, filtered through a short column of silica and evaporated. The residue was subjected to preparative HPLC. The appropriate fractions were pooled and freeze-dried to provide the title compound as a white solid (29 mg).

¹H-NMR (DMSO-d₆): δ 9.77 (t, J=6.1 Hz, 1H), 8.78 (d, J=1.3 Hz, 1H), 8.01-7.69 (m, 6H), 7.55 (d, J=8.3 Hz, 2H), 4.63-4.58 (m, 2H), 3.17 (s, 3H), 2.78 (s, 1.4H), 2.77 (s, 1.6H), 2.08 (s, 1.4H), 2.08 (s, 1.6H);

APCI-MS m/z: 527.3 [MH⁺].

### EXAMPLE 91

### 6-Methyl-5-(methylsulfonyl)-N-[4-(methylsulfonyl)benzyl]-2-oxo-1-[3-(trifluoromethyl)phenyl]-1,2-dihydropyridine-3-carboxamide

6-Methyl-5-(methylsulfinyl)-N-[4-(methylsulfonyl)benzyl]-2-oxo-1-[3-(trifluoromethyl)phenyl]-1,2-dihydropyridine-3-carboxamide (Example 90, 51 mg, 0.1 mmol) was dissolved in acetic acid (5 ml). Hydrogen peroxide (3 ml of 35% solution in water) was added and the mixture was stirred at 60° C. overnight. The reaction mixture was directly purified using semi-preparative HPLC. The appropriate fractions were pooled and freeze-dried to provide the title compound as a white solid (32 mg).

¹H-NMR (DMSO-d₆): δ 9.63 (t, J=6.1 Hz, 1H), 8.78 (s, 1H), 8.01-7.76 (m, 6H), 7.54 (d, J=8.4 Hz, 2H), 4.59 (d, J=6.1 Hz, 2H), 3.29 (s, 3H), 3.17 (s, 3H), 2.38 (s, 3H);

APCI-MS m/z: 542.9 [MH⁺].

### EXAMPLE 92

### 5-(Benzylsulfinyl)-6-methyl-N-[4-(methylsulfonyl)benzyl]-2-oxo-1-[3-(trifluoromethyl)phenyl]-1,2-dihydropyridine-3-carboxamide

The title compound was prepared using a procedure analogous to that described for Example 90.

¹H-NMR (DMSO-d₆): δ 9.73 (t, J=6.0 Hz, 1H), 8.60 (d, J=4.4 Hz, 1H), 7.96-7.65 (m, 6H), 7.56 (d, J=8.3 Hz, 2H), 7.41-7.19 (m, 5H), 4.60 (d, J=6.0 Hz, 2H), 4.47-4.35 (m, 1H), 4.18-4.09 (m, 1H), 3.18 (s, 3H), 1.54 (s, 1.5H), 1.54 (s, 1.5H);

APCI-MS m/z: 603.4 [MH⁺].

### EXAMPLE 93

### 5-(Ethylsulfinyl)-6-methyl-N-[4-(methylsulfonyl)benzyl]-2-oxo-1-[3-(trifluoromethyl)phenyl]-1,2-dihydropyridine-3-carboxamide

The title compound was prepared using a procedure analogous to that described for Example 90.

¹H-NMR (DMSO-d₆): δ 9.77 (t, J=6.1 Hz, 1H), 8.69 (s, 1H), 8.01-7.74 (m, 6H), 7.55 (d, J=8.4 Hz, 2H), 4.63-4.57 (m, 2H), 3.17 (s, 3H), 3.06-2.79 (m, 2H), 2.07 (s, 1.5H), 2.06 (s, 1.5H), 1.15 (t, J=7.3 Hz, 3H);

APCI-MS m/z: 541.3 [MH⁺].

### EXAMPLE 94

### Methyl 3-({2-methyl-5-({[4-(methylsulfonyl)benzyl]amino}carbonyl)-6-oxo-1-[3-(trifluoromethyl)phenyl]-1,6-dihydropyridin-3-yl}sulfinyl)propanoate

The title compound was prepared using a procedure analogous to that described for Example 90.

¹H-NMR (DMSO-d₆): δ 9.76 (t, J=6.1 Hz, 1H), 8.68 (s, 0.5H), 8.68 (s, 0.5H), 7.98-7.71 (m, 6H), 7.55 (d, J=8.3 Hz, 2H), 4.63-4.57 (m, 2H), 3.60 (s, 1.5H), 3.59 (s, 1.5H), 3.27-3.01 (m, 5H), 2.84-2.61 (m, 2H), 2.06 (s, 3H);

APCI-MS m/z: 599.1 [MH⁺].

### EXAMPLE 95

### 5-(Cyclohexylsulfinyl)-6-methyl-N-[4-(methylsulfonyl)benzyl]-2-oxo-1-[3-(trifluoromethyl)phenyl]-1,2-dihydropyridine-3-carboxamide

The title compound was prepared using a procedure analogous to that described for Example 90.

¹H-NMR (DMSO-d₆): δ 9.79 (t, J=6.1 Hz, 1H), 8.64 (s, 1H), 8.06-7.75 (m, 6H), 7.55 (d, J=8.1 Hz, 2H), 4.67-4.50 (m, 2H), 3.17 (s, 3H), 2.84-2.68 (m, 1H), 2.06 (s, 3H), 1.95-1.70 (m, 4H), 1.68-1.57 (m, 2H), 1.50-1.09 (m, 4H);

APCI-MS m/z: 595.1 [MH⁺].

### EXAMPLE 96

### 5-(Cyclopropylsulfonyl)-N-[4-(cyclopropylsulfonyl)benzyl]-6-methyl-2-oxo-1-[3-(trifluoromethyl)phenyl]-1,2-dihydropyridine-3-carboxamide

### a) Sodium Cyclopropanesulfinate

The subtitle compound was obtained as a white solid, starting from cyclopropanesulfonyl chloride, using an analogous synthetic procedure to that described in Helvetica Chimica Acta, vol. 86 (2003), 65-81.

¹H NMR (CD₃OD): δ 1.87 (1H, tt, J 8.2, 5.0 Hz); 0.75 (2H, m; 0.61 (2H, m).

### b) 5-(Cyclopropylsulfonyl)-N-[4-(cyclopropylsulfonyl)benzyl]-6-methyl-2-oxo-1-[3-(trifluoromethyl)phenyl]-1,2-dihydropyridine-3-carboxamide

A mixture of N-[4-(cyclopropylsulfonyl)benzyl]-5-iodo-6-methyl-2-oxo-1-[3-(trifluoromethyl)phenyl]-1,2-dihydropyridine-3-carboxamide (SM28, 180.4 mg, 0.29 mmol), copper(I) iodide (69.9 mg, 0.37 mmol), sodium cyclopropanesulfinate (Example 96a, 75.4 mg, 0.59 mmol) and DMF (2 ml) was stirred at 100° C. for 1 h. The reaction mixture was cooled and partitioned between ethyl acetate and water. The organic layer was washed with water, brine, dried over sodium sulfate, filtered and concentrated in vacuum. The residue was purified by preparative HPLC to give the title compound as white solid (12 mg, 7%).

1H NMR (CDCl₃): δ 9.60 (1H, t, J=5.8 Hz); 9.09 (1H, s); 7.90-7.76 (4H, m); 7.53 (1H, s); 7.49 (2H, d, J=8.3 Hz); 7.44 (1H, d, J=8.0 Hz); 4.69 (2H, m); 2.60 (1H, m); 2.42 (1H, m); 1.44 (2H, m); 1.33 (2H, m); 1.18 (2H, m); 1.02 (2H, m); 2.54 (3H, s);

APCI-MS m/z: 595.4 [MH⁺].

### Human Neutrophil Elastase Quenched-FRET Assay

The assay uses Human Neutrophil Elastase (HNE) purified from serum (Calbiochem art. 324681; Ref. Baugh, R. J. et al., 1976, Biochemistry. 15, 836-841). HNE was stored in 50 mM sodium acetate (NaOAc), 200 mM sodium chloride (NaCl), pH 5.5 with added 30% glycerol at −20° C. The protease substrate used was Elastase Substrate V Fluorogenic, MeOSuc-AAPV-AMC (Calbiochem art. 324740; Ref. Castillo, M. J. et al., 1979, Anal. Biochem. 99, 53-64). The substrate was stored in dimethyl sulphoxide (DMSO) at −20° C. The assay additions were as follows: Test compounds and controls were added to black 96-well flat-bottom plates (Greiner 655076), 1 μL in 100% DMSO, followed by 30 μL HNE in assay buffer with 0.01% Triton (trade mark) X-100 detergent. The assay buffer constitution was: 100 mM Tris(hydroxymethyl)aminomethane (TRIS) (pH 7.5) and 500 mM NaCl. The enzyme and the compounds were incubated at room temperature for 15 minutes. Then 30 μl substrate in assay buffer was added. The assay was incubated for 30 minutes at room temperature. The concentrations of HNE enzyme and substrate during the incubation were 1.7 nM and 100 μM, respectively. The assay was then stopped by adding 60 μl stop solution (140 mM acetic acid, 200 mM sodium monochloroacetate, 60 mM sodium acetate, pH 4.3). Fluorescence was measured on a Wallac 1420 Victor 2 instrument at settings: Excitation 380 nm, Emission 460 nm. IC₅₀ values were determined using Xlfit curve fitting using model 205.

When tested in the above screen, the compounds of the Examples gave IC₅₀ values for inhibition of human neutrophil elastase activity of less than 30 μM (micromolar), indicating that the compounds of the invention are expected to possess useful therapeutic properties. Specimen results are shown in the following Table:

| | Inhibition of Human Neutrophil Elastase IC₅₀ |
|---|---|
| Compound of | (micromolar, μM) |
| Example 27 | 0.009 |
| Example 49 | 0.004 |
| Example 54 | 0.0005 |
| Example 59 | 0.014 |
| Example 86 | 0.045 |

1. A compound of formula (I) 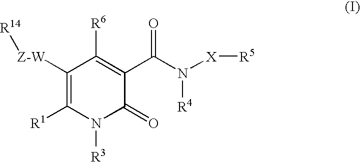
wherein
R¹ represents hydrogen or C₁-C₆ alkyl;
W represents S(O)ₘ wherein m represents an integer 0, 1 or 2;
Z represents a single bond, —CH₂ or —NR²⁵—;
R¹⁴ represents a hydrogen atom or OH or a group selected from C₁-C₆ alkyl and a saturated or unsaturated 3- to 10-membered ring system optionally comprising at least one ring heteroatom selected from nitrogen, oxygen and sulphur; each group being optionally substituted with at least one substituent selected from phenyl, C₁-C₆ alkoxycarbonyl, halogen, C₁-C₄ alkyl, C₁-C₄ alkoxy, CN, OH, NO₂, C₁-C₃ alkyl substituted by one or more F atoms, C₁-C₃ alkoxy substituted by one or more F atoms, NR¹²R¹³, C≡CR³⁰, CONR³¹R³², CHO, C₂-C₄ alkanoyl, S(O)ₚR³³ and OSO₂R³⁴;
R¹² and R¹³ independently represent H, C₁-C₆ alkyl, formyl or C₂-C₆ alkanoyl; or the group —NR¹²R¹³ together represents a 5 to 7 membered azacyclic ring optionally incorporating one further heteroatom selected from O, S and NR²⁶;
R³⁰ represents H, C₁-C₃ alkyl, Si(CH₃)₃ or phenyl;
R³³ and R³⁴ independently represent H or C₁-C₃ alkyl; said alkyl being optionally substituted by one or more F atoms;
R⁶ represents H or F;
R³ represents phenyl or a five- or six-membered heteroaromatic ring containing 1 to 3 heteroatoms independently selected from O, S and N; said ring being optionally substituted with at least one substituent selected from halogen, C₁-C₆ alkyl, cyano, C₁-C₆ alkoxy, nitro, methylcarbonyl, NR³⁵R³⁶, C₁-C₃ alkyl substituted by one or more F atoms or C₁-C₃ alkoxy substituted by one or more F atoms;
R³⁵ and R³⁶ independently represent H or C₁-C₃ alkyl; said alkyl being optionally further substituted by one or more F atoms;
R⁴ represents hydrogen or C₁-C₆ alkyl optionally substituted with at least one substituent selected from fluoro, hydroxyl and C₁-C₆ alkoxy;
X represents a single bond, O, NR²⁴ or a group —C₁-C₆ alkylene-Y—, wherein Y represents a single bond, oxygen atom, NR²⁴ or S(O)_{w}; and said alkylene being optionally further substituted by OH, halogen, CN, NR³⁷R³⁸, C₁-C₃alkoxy, CONR³⁹R⁴⁰, SO₂R⁴¹ and SO₂NR⁴²R⁴³;
or R⁴ and X are joined together such that the group —NR⁴X together represents a 5 to 7 membered azacyclic ring optionally incorporating one further heteroatom selected from O, S and NR⁴⁴; said ring being optionally substituted by C₁-C₆ alkyl or NR⁴⁵R⁴⁶; said alkyl being optionally further substituted by OH;
either R⁵ represents a monocyclic ring system selected from
i) phenoxy,
ii) phenyl,
iii) a 5- or 6-membered heteroaromatic ring comprising at least one ring heteroatom selected from nitrogen, oxygen and sulphur,
iv) a saturated or partially unsaturated C₃-C₆ hydrocarbyl ring, or
v) a saturated or partially unsaturated 4- to 7-membered heterocyclic ring comprising at least one ring heteroatom selected from oxygen, S(O)ᵣ and NR²⁰, wherein at least one of the ring carbon atoms may be optionally replaced by a carbonyl group,
or R⁵ represents a bicyclic ring system in which the two rings are independently selected from the monocyclic ring systems defined in ii), iii), iv) and v) above, wherein the two rings are either fused together, bonded directly to one another or are separated from one another by a linker group selected from oxygen, S(O)ₜ or C₁-C₆ alkylene optionally comprising one or more internal or terminal heteroatoms selected from oxygen, sulphur and NR²⁷ and being optionally substituted by at least one substituent selected from hydroxyl, oxo and C₁-C₆ alkoxy,
the monocyclic or bicyclic ring system being optionally substituted by at least one substituent selected from oxygen, CN, OH, C₁-C₆ alkyl, C₁-C₆ alkoxy, halogen, NR⁴⁷R⁴⁸, NO₂, OSO₂R⁴⁹, CO₂R⁵⁰, C(═NH)NH₂, C(O)NR⁵¹R⁵², C(S)NR⁵³R⁵⁴, SC(═NH)NH₂, NR⁵⁵C(═NH)NH₂, S(O)ᵥR²¹, SO₂NR⁵⁶R⁵⁷, C₁-C₃ alkoxy substituted by one or more F atoms and C₁-C₃ alkyl substituted by SO₂R⁵⁸ or by one or more F atoms; said C₁-C₆ alkyl being optionally further substituted with at least one substituent selected from cyano, hydroxyl, C₁-C₆ alkoxy, C₁-C₆ alkylthio and —C(O)NR²²R²³;
or R⁵ may also represent H;
R²⁰ represents hydrogen, C₁-C₆ alkyl, C₁-C₆ alkylcarbonyl or C₁-C₆ alkoxycarbonyl;
R²¹ represents hydrogen, C₁-C₆ alkyl or C₃-C₈ cycloalkyl; said alkyl or cycloalkyl group being optionally further substituted by one or more substituents selected independently from OH, CN, C₁-C₃ alkoxy and CONR⁵⁹R⁶⁰;
R³⁷ and R³⁸ independently represent H, C₁-C₆ alkyl, formyl or C₂-C₆ alkanoyl;
R⁴⁷ and R⁴⁸ independently represent H, C₁-C₆ alkyl, formyl, C₂-C₆ alkanoyl, S(O)_{q}R⁶¹ or SO₂NR⁶²R⁶³; said alkyl group being optionally further substituted by halogen, CN, C₁-C₄ alkoxy or CONR⁶⁴R⁶⁵;
R⁴¹ and R⁶¹ independently represent H, C₁-C₆ alkyl or C₃-C₆ cycloalkyl;
p is 0, 1 or 2;
q is 0, 1 or 2;
r is 0, 1 or 2;
t is 0, 1 or 2;
w is 0, 1 or 2;
v is 0, 1 or 2;
R²², R²³, R²⁴, R²⁵, R²⁶, R²⁷, R³¹, R³², R³⁹, R⁴⁰, R⁴², R⁴³, R⁴⁴, R⁴⁵, R⁴⁶, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶², R⁶³, R⁶⁴ and R⁶⁵ each independently represent hydrogen or C₁-C₆ alkyl;
or a pharmaceutically acceptable salt thereof.

2. The compound according to claim 1, wherein R¹⁴ represents phenyl optionally substituted by one or two substituents independently selected from CN, F, Cl, Br, CF₃, NO₂ and C≡CH.

3. The compound according to claim 1, wherein Z represents a single bond.

4. The compound according to claim 1, wherein R³ represents a phenyl group substituted with a trifluoromethyl substituent.

5. The compound according to claim 1, wherein R⁵ represents phenyl or pyridinyl substituted by —S(O)ᵥR²¹ wherein v represents the integer 2.

6. The compound according to claim 1, wherein R⁵ represents H.

7. A compound of formula (I) as defined in claim 1 selected from:
N-Cyclopropyl-5-[(4-methoxyphenyl)sulfinyl]-6-methyl-2-oxo-1-[3-(trifluoromethyl)phenyl]-1,2-dihydropyridine-3-carboxamide;
2-Oxo-N-[3-(2-oxopyrrolidin-1-yl)propyl]-5-(phenylsulfinyl)-1-[3-(trifluoromethyl)phenyl]-1,2-dihydropyridine-3-carboxamide;
5-[(4-Bromophenyl)sulfinyl]-6-methyl-N-[4-(methylsulfonyl)benzyl]-2-oxo-1-[3-(trifluoromethyl)phenyl]-1,2-dihydropyridine-3-carboxamide;
5-[(2,4-Dimethoxybenzyl)sulfinyl]-6-methyl-N-[4-(methylsulfonyl)benzyl]-2-oxo-1-[3-(trifluoromethyl)phenyl]-1,2-dihydropyridine-3-carboxamide;
5-[(4-Cyanophenyl)sulfinyl]-N-cyclopropyl-6-methyl-2-oxo-1-[3-(trifluoromethyl)phenyl]-1,2-dihydropyridine-3-carboxamide;
N-{[5-(Cyclopropylsulfonyl)pyridin-2-yl]methyl}-2-oxo-5-(phenylsulfinyl)-1-[3-(trifluoromethyl)phenyl]-1,2-dihydropyridine-3-carboxamide;
6-Methyl-5-(methylsulfinyl)-N-{[5-(methylsulfonyl)pyridin-2-yl]methyl}-2-oxo-1-[3-(trifluoromethyl)phenyl]-1,2-dihydropyridine-3-carboxamide;
N-Cyclopropyl-5-[(3-methoxyphenyl)sulfinyl]-6-methyl-2-oxo-1-[3-(trifluoromethyl)phenyl]-1,2-dihydropyridine-3-carboxamide;
N-Cyclopropyl-5-[(2-methoxyphenyl)sulfinyl]-6-methyl-2-oxo-1-[3-(trifluoromethyl)phenyl]-1,2-dihydropyridine-3-carboxamide;
5-[(4-Cyanophenyl)sulfinyl]-N-[(2S)-2-hydroxypropyl]-6-methyl-2-oxo-1-[3-(trifluoromethyl)phenyl]-1,2-dihydropyridine-3-carboxamide;
5-[(4-Cyanophenyl)sulfinyl]-6-methyl-2-oxo-1-[3-(trifluoromethyl)phenyl]-1,2-dihydropyridine-3-carboxamide;
5-[(4-Cyanophenyl)sulfinyl]-6-methyl-N-[4-(methylsulfonyl)benzyl]-2-oxo-1-[3-(trifluoromethyl)phenyl]-1,2-dihydropyridine-3-carboxamide;
5-[(2-Cyanoethyl)sulfinyl]-6-methyl-N-[4-(methylsulfonyl)benzyl]-2-oxo-1-[3-(trifluoromethyl)phenyl]-1,2-dihydropyridine-3-carboxamide;
5-[(4-Cyanophenyl)sulfinyl]-N-cyclopropyl-1-(3,5-difluorophenyl)-6-methyl-2-oxo-1,2-dihydropyridine-3-carboxamide;
5-[(4-Cyanophenyl)sulfinyl]-N-{[5-(ethylsulfonyl)pyridin-2-yl]methyl}-6-methyl-2-oxo-1-[3-(trifluoromethyl)phenyl]-1,2-dihydropyridine-3-carboxamide;
5-[(4-Cyanophenyl)sulfinyl]-1-(3,5-difluorophenyl)-N-{[5-(ethylsulfonyl)pyridin-2-yl]methyl}-6-methyl-2-oxo-1,2-dihydropyridine-3-carboxamide;
5-[(4-Cyanophenyl)sulfinyl]-1-(3,5-dichlorophenyl)-N-{[5-(ethylsulfonyl)pyridin-2-yl]methyl}-6-methyl-2-oxo-1,2-dihydropyridine-3-carboxamide;
5-[(4-Cyanophenyl)sulfinyl]-N,6-dimethyl-2-oxo-1-[3-(trifluoromethyl)phenyl]-1,2-dihydropyridine-3-carboxamide;
5-[(4-Cyanophenyl)sulfinyl]-1-(3,5-dichlorophenyl)-N,6-dimethyl-2-oxo-1,2-dihydropyridine-3-carboxamide;
5-[(4-Cyanophenyl)sulfinyl]-1-(3,5-difluorophenyl)-N-[2-(1H-imidazol-4-yl)ethyl]-6-methyl-2-oxo-1,2-dihydropyridine-3-carboxamide;
5-[(4-Cyanophenyl)sulfinyl]-1-(3,5-difluorophenyl)-6-methyl-N-(2-morpholin-4-ylethyl)-2-oxo-1,2-dihydropyridine-3-carboxamide;
5-[(4-Cyanophenyl)sulfinyl]-1-(3,5-difluorophenyl)-N,6-dimethyl-2-oxo-1,2-dihydropyridine-3-carboxamide;
5-[(4-Cyanophenyl)sulfinyl]-6-methyl-N-[(3-methylisoxazol-5-yl)methyl]-2-oxo-1-[3-(trifluoromethyl)phenyl]-1,2-dihydropyridine-3-carboxamide;
N-Cyclopropyl-5-[(4-hydroxyphenyl)sulfinyl]-6-methyl-2-oxo-1-[3-(trifluoromethy)phenyl]-1,2-dihydropyridine-3-carboxamide;
5-[(4-Cyanophenyl)sulfinyl]-N-[3-(1H-imidazol-1-yl)propyl]-6-methyl-2-oxo-1-[3-(trifluoromethyl)phenyl]-1,2-dihydropyridine-3-carboxamide;
5-[(4-Cyanophenyl)sulfinyl]-6-methyl-2-oxo-N-[3-(1H-1,2,3-triazol-1-yl)propyl]-1-[3-(trifluoromethyl)phenyl]-1,2-dihydropyridine-3-carboxamide;
5-[(4-Cyanophenyl)sulfinyl]-N-[(1-hydroxycyclopropyl)methyl]-6-methyl-2-oxo-1-[3-(trifluoromethyl)phenyl]-1,2-dihydropyridine-3-carboxamide;
1-(3-Cyanophenyl)-5-[(4-cyanophenyl)sulfinyl]-6-methyl-N-{[5-(methylsulfonyl)pyridin-2-yl]methyl}-2-oxo-1,2-dihydropyridine-3-carboxamide;
5-[(4-Cyanophenyl)sulfinyl]-N-(2-methoxyethyl)-6-methyl-2-oxo-1-[3-(trifluoromethyl)phenyl]-1,2-dihydropyridine-3-carboxamide;
5-[(4-Cyanophenyl)sulfinyl]-N-(2-hydroxy-2-methylpropyl)-6-methyl-2-oxo-1-[3-(trifluoromethyl)phenyl]-1,2-dihydropyridine-3-carboxamide;
5-[(4-Chlorophenyl)sulfinyl]-6-methyl-N-[4-(methylsulfonyl)benzyl]-2-oxo-1-[3-(trifluoromethyl)phenyl]-1,2-dihydropyridine-3-carboxamide;
6-Methyl-5-[(4-methylphenyl)sulfinyl]-N-[4-(methylsulfonyl)benzyl]-2-oxo-1-[3-(trifluoromethyl)phenyl]-1,2-dihydropyridine-3-carboxamide;
6-Methyl-N-[4-(methylsulfonyl)benzyl]-5-[(4-nitrophenyl)sulfinyl]-2-oxo-1-[3-(trifluoromethyl)phenyl]-1,2-dihydropyridine-3-carboxamide;
6-Methyl-N-[4-(methylsulfonyl)benzyl]-2-oxo-1-[3-(trifluoromethyl)phenyl]-5-{[4-(trifluoromethyl)phenyl]sulfinyl}-1,2-dihydropyridine-3-carboxamide;
5-{[4-(Acetylamino)phenyl]sulfinyl}-6-methyl-N-[4-(methylsulfonyl)benzyl]-2-oxo-1-[3-(trifluoromethyl)phenyl]-1,2-dihydropyridine-3-carboxamide;
5-[(4-Ethylphenyl)sulfinyl]-6-methyl-N-[4-(methylsulfonyl)benzyl]-2-oxo-1-[3-(trifluoromethyl)phenyl]-1,2-dihydropyridine-3-carboxamide;
5-[(4-Fluorophenyl)sulfinyl]-6-methyl-N-[4-(methylsulfonyl)benzyl]-2-oxo-1-[3-(trifluoromethyl)phenyl]-1,2-dihydropyridine-3-carboxamide;
5-[(4-Cyanophenyl)sulfinyl]-6-methyl-1-(3-methylphenyl)-N-{[5-(methylsulfonyl)pyridin-2-yl]methyl}-2-oxo-1,2-dihydropyridine-3-carboxamide;
5-[(4-Cyanophenyl)sulfinyl]-N-ethyl-6-methyl-2-oxo-1-[3-(trifluoromethyl)phenyl]-1,2-dihydropyridine-3-carboxamide;
5-[(4-Chlorophenyl)sulfinyl]-N,6-dimethyl-2-oxo-1-[3-(trifluoromethyl)phenyl]-1,2-dihydropyridine-3-carboxamide;
N-Ethyl-5-[(4-fluorophenyl)sulfinyl]-6-methyl-2-oxo-1-[3-(trifluoromethyl)phenyl]-1,2-dihydropyridine-3-carboxamide;
5-[(4-Fluorophenyl)sulfinyl]-N,6-dimethyl-2-oxo-1-[3-(trifluoromethyl)phenyl]-1,2-dihydropyridine-3-carboxamide;
5-[(4-Bromophenyl)sulfinyl]-N,6-dimethyl-2-oxo-1-[3-(trifluoromethyl)phenyl]-1,2-dihydropyridine-3-carboxamide;
5-[(4-Cyanophenyl)sulfinyl]-N-(2-hydroxyethyl)-6-methyl-2-oxo-1-[3-(trifluoromethyl)phenyl]-1,2-dihydropyridine-3-carboxamide;
5-[(4-Cyanophenyl)sulfinyl]-N-(cyclopropylmethyl)-6-methyl-2-oxo-1-[3-(trifluoromethyl)phenyl]-1,2-dihydropyridine-3-carboxamide;
N-Methyl-2-oxo-5-(phenylsulfinyl)-1-[3-(trifluoromethyl)phenyl]-1,2-dihydropyridine-3-carboxamide;
N-(Cyanomethyl)-5-[(4-cyanophenyl)sulfinyl]-6-methyl-2-oxo-1-[3-(trifluoromethyl)phenyl]-1,2-dihydropyridine-3-carboxamide;
5-[(4-Cyanophenyl)sulfinyl]-N-[2-(1H-imidazol-4-yl)ethyl]-6-methyl-2-oxo-1-[3-(trifluoromethyl)phenyl]-1,2-dihydropyridine-3-carboxamide;
5-[(4-Cyanophenyl)sulfinyl]-N-(2-hydroxypropyl)-6-methyl-2-oxo-1-[3-(trifluoromethyl)phenyl]-1,2-dihydropyridine-3-carboxamide;
5-[(4-Cyanophenyl)sulfinyl]-6-methyl-N-(2-morpholin-4-ylethyl)-2-oxo-1-[3-(trifluoromethyl)phenyl]-1,2-dihydropyridine-3-carboxamide;
5-[(4-Cyanophenyl)sulfinyl]-N-(2-hydroxy-1,1-dimethylethyl)-6-methyl-2-oxo-1-[3-(trifluoromethyl)phenyl]-1,2-dihydropyridine-3-carboxamide;
5-[(4-Cyanophenyl)sulfinyl]-N,6-dimethyl-2-oxo-1-[3-(trifluoromethyl)phenyl]-1,2-dihydropyridine-3-carboxamide;
5-[(4-Cyanophenyl)sulfinyl]-N-[(2R)-2-hydroxypropyl]-6-methyl-2-oxo-1-[3-(trifluoromethyl)phenyl]-1,2-dihydropyridine-3-carboxamide;
5-[(4-Cyanophenyl)sulfinyl]-6-methyl-2-oxo-N-[3-(2-oxopyrrolidin-1-yl)propyl]-1-[3-(trifluoromethyl)phenyl]-1,2-dihydropyridine-3-carboxamide;
5-[(4-Cyanophenyl)sulfinyl]-N-(2-methoxypropyl)-6-methyl-2-oxo-1-[3-(trifluoromethyl)phenyl]-1,2-dihydropyridine-3-carboxamide;
6-Methyl-5-(methylsulfonyl)-N-{[5-(methylsulfonyl)pyridin-2-yl]methyl}-2-oxo-1-[3-(trifluoromethyl)phenyl]-1,2-dihydropyridine-3-carboxamide;
2-Oxo-N-[3-(2-oxopyrrolidin-1-yl)propyl]-5-(phenylsulfonyl)-1-[3-(trifluoromethyl)phenyl]-1,2-dihydropyridine-3-carboxamide;
5-[(4-Cyanophenyl)sulfonyl]-N,6-dimethyl-2-oxo-1-[3-(trifluoromethyl)phenyl]-1,2-dihydropyridine-3-carboxamide;
5-{[4-(Acetylamino)phenyl]sulfonyl}-6-methyl-N-[4-(methylsulfonyl)benzyl]-2-oxo-1-[3-(trifluoromethyl)phenyl]-1,2-dihydropyridine-3-carboxamide;
5-[(4-Ethylphenyl)sulfonyl]-6-methyl-N-[4-(methylsulfonyl)benzyl]-2-oxo-1-[3-(trifluoromethyl)phenyl]-1,2-dihydropyridine-3-carboxamide;
5-[(4-Cyanophenyl)sulfonyl]-N,6-dimethyl-2-oxo-1-[3-(trifluoromethyl)phenyl]-1,2-dihydropyridine-3-carboxamide;
5-[(4-Cyanophenyl)sulfonyl]-N-(2-hydroxy-1,1-dimethylethyl)-6-methyl-2-oxo-1-[3-(trifluoromethyl)phenyl]-1,2-dihydropyridine-3-carboxamide;
N-[(3-Cyclopropylisoxazol-5-yl)methyl]-6-methyl-5-(methylsulfonyl)-2-oxo-1-[3-(trifluoromethyl)phenyl]-1,2-dihydropyridine-3-carboxamide;
5-[(6-Cyanopyridin-3-yl)sulfonyl]-N,6-dimethyl-2-oxo-1-[3-(trifluoromethyl)phenyl]-1,2-dihydropyridine-3-carboxamide;
6-Methyl-N-[4-(methylsulfonyl)benzyl]-2-oxo-1-[3-(trifluoromethyl)phenyl]-5-({4-[(trimethylsilyl)ethynyl]phenyl}sulfinyl)-1,2-dihydropyridine-3-carboxamide;
5-[(4-Ethynylphenyl)sulfinyl]-6-methyl-N-[4-(methylsulfonyl)benzyl]-2-oxo-1-[3-(trifluoromethyl)phenyl]-1,2-dihydropyridine-3-carboxamide;
6-Methyl-N-[4-(methylsulfonyl)benzyl]-2-oxo-5-{[4-(phenylethynyl)phenyl]sulfinyl}-1-[3-(trifluoromethyl)phenyl]-1,2-dihydropyridine-3-carboxamide;
6-Methyl-N-[4-(methylsulfonyl)benzyl]-2-oxo-5-[(4-prop-1-yn-1-ylphenyl)sulfinyl]-1-[3-(trifluoromethyl)phenyl]-1,2-dihydropyridine-3-carboxamide;
5-[(5-Cyanopyridin-2-yl)sulfinyl]-N,6-dimethyl-2-oxo-1-[3-(trifluoromethyl)phenyl]-1,2-dihydropyridine-3-carboxamide;
6-({2-Methyl-5-(methylcarbamoyl)-6-oxo-1-[3-(trifluoromethyl)phenyl]-1,6-dihydropyridin-3-yl}sulfinyl)nicotinamide;
5-[(5-Chloropyridin-2-yl)sulfinyl]-N,6-dimethyl-2-oxo-1-[3-(trifluoromethyl)phenyl]-1,2-dihydropyridine-3-carboxamide;
5-[(5-Bromopyridin-2-yl)sulfinyl]-N,6-dimethyl-2-oxo-1-[3-(trifluoromethyl)phenyl]-1,2-dihydropyridine-3-carboxamide;
5-[(5-Cyanopyridin-2-yl)sulfinyl]-6-methyl-N-[4-(methylsulfonyl)benzyl]-2-oxo-1-[3-(trifluoromethyl)phenyl]-1,2-dihydropyridine-3-carboxamide;
5-[(5-Bromopyrimidin-2-yl)sulfinyl]-N,6-dimethyl-2-oxo-1-[3-(trifluoromethyl)phenyl]-1,2-dihydropyridine-3-carboxamide;
5-[(6-Bromopyridazin-3-yl)sulfinyl]-N,6-dimethyl-2-oxo-1-[3-(trifluoromethyl)phenyl]-1,2-dihydropyridine-3-carboxamide;
5-[(6-Cyanopyridin-3-yl)sulfinyl]-N,6-dimethyl-2-oxo-1-[3-(trifluoromethyl)phenyl]-1,2-dihydropyridine-3-carboxamide;
5-[(5-Cyano-2-thienyl)sulfinyl]-N,6-dimethyl-2-oxo-1-[3-(trifluoromethyl)phenyl]-1,2-dihydropyridine-3-carboxamide;
5-(1H-Imidazol-2-ylsulfinyl)-6-methyl-N-[4-(methylsulfonyl)benzyl]-2-oxo-1-[3-(trifluoromethyl)phenyl]-1,2-dihydropyridine-3-carboxamide;
6-Methyl-5-[(methylamino)sulfonyl]-N-[4-(methylsulfonyl)benzyl]-2-oxo-1-[3-(trifluoromethyl)phenyl]-1,2-dihydropyridine-3-carboxamide;
5-(Anilinosulfonyl)-6-methyl-N-[4-(methylsulfonyl)benzyl]-2-oxo-1-[3-(trifluoromethyl)phenyl]-1,2-dihydropyridine-3-carboxamide;
6-Methyl-N-[4-(methylsulfonyl)benzyl]-5-{[(2-morpholin-4-ylethyl)amino]sulfonyl}-2-oxo-1-[3-(trifluoromethyl)phenyl]-1,2-dihydropyidine-3-carboxamide;
5-{[(2-Cyanoethyl)(methyl)amino]sulfonyl}-6-methyl-N-[4-(methylsulfonyl)benzyl]-2-oxo-1-[3-(trifluoromethyl)phenyl]-1,2-dihydropyridine-3-carboxamide;
6-Methyl-N-[4-(methylsulfonyl)benzyl]-5-{[(6-morpholin-4-ylpyridin-3-yl)amino]sulfonyl}-2-oxo-1-[3-(trifluoromethyl)phenyl]-1,2-dihydropyridine-3-carboxamide;
6-Methyl-N-[4-(methylsulfonyl)benzyl]-5-(morpholin-4-ylsulfonyl)-2-oxo-1-[3-(trifluoromethyl)phenyl]-1,2-dihydropyridine-3-carboxamide;
6-Methyl-N-[4-(methylsulfonyl)benzyl]-2-oxo-5-[(pyridin-3-ylamino)sulfonyl]-1-[3-(trifluoromethyl)phenyl]-1,2-dihydropyridine-3-carboxamide;
2-Methyl-5-({[4-(methylsulfonyl)benzyl]amino}carbonyl)-6-oxo-1-[3-(trifluoromethyl)phenyl]-1,6-dihydropyridine-3-sulfonic acid;
6-Methyl-N-[4-(methylsulfonyl)benzyl]-2-oxo-5-(phenylthio)-1-[3-(trifluoromethyl)phenyl]-1,2-dihydropyridine-3-carboxamide;
6-Methyl-N-[4-(methylsulfonyl)benzyl]-2-oxo-5-(phenylsulfinyl)-1-[3-(trifluoromethyl)phenyl]-1,2-dihydropyridine-3-carboxamide;
6-Methyl-N-[4-(methylsulfonyl)benzyl]-2-oxo-5-(phenylsulfonyl)-1-[3-(trifluoromethyl)phenyl]-1,2-dihydropyridine-3-carboxamide;
6-Methyl-5-(methylsulfinyl)-N-[4-(methylsulfonyl)benzyl]-2-oxo-1-[3-(trifluoromethyl)phenyl]-1,2-dihydropyridine-3-carboxamide;
6-Methyl-5-(methylsulfonyl)-N-[4-(methylsulfonyl)benzyl]-2-oxo-1-[3-(trifluoromethyl)phenyl]-1,2-dihydropyridine-3-carboxamide;
5-(Benzylsulfinyl)-6-methyl-N-[4-(methylsulfonyl)benzyl]-2-oxo-1-[3-(trifluoromethyl)phenyl]-1,2-dihydropyridine-3-carboxamide;
5-(Ethylsulfinyl)-6-methyl-N-[4-(methylsulfonyl)benzyl]-2-oxo-1-[3-(trifluoromethyl)phenyl]-1,2-dihydropyridine-3-carboxamide;
Methyl 3-({2-methyl-5-({[4-(methylsulfonyl)benzyl]amino}carbonyl)-6-oxo-1-[3-(trifluoromethyl)phenyl]-1,6-dihydropyridin-3-yl}sulfinyl)propanoate;
5-(Cyclohexylsulfinyl)-6-methyl-N-[4-(methylsulfonyl)benzyl]-2-oxo-1-[3-(trifluoromethyl)phenyl]-1,2-dihydropyridine-3-carboxamide; and
5-(Cyclopropylsulfonyl)-N-[4-(cyclopropylsulfonyl)benzyl]-6-methyl-2-oxo-[3-(trifluoromethyl)phenyl]-1,2-dihydropyridine-3-carboxamide;
and pharmaceutically acceptable salts thereof.

8. A process for the preparation of a compound of formula (I) or a pharmaceutically acceptable salt thereof as defined in claim 1 which comprises,
(a) reacting a compound of formula (II) 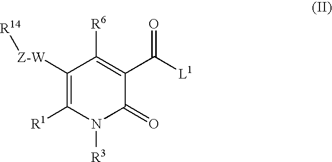
wherein L¹ represents a leaving group and R¹, R³, R⁶, R¹⁴, W and Z are as defined in claim 1,
with a compound of formula 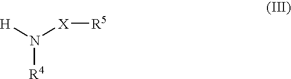
wherein X, R⁴ and R⁵ are as defined in claim 1; or
(b) when W represents —S— and Z represents a single bond or —CH₂—, reacting a compound of formula (IV) 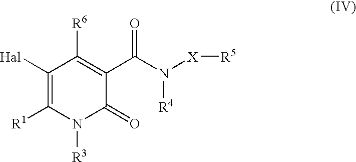
wherein Hal represents a halogen atom and X, R¹, R³, R⁴, R⁵ and R⁶ are as defined in claim 1,
with a nucleophile R¹⁴-Z-S-M wherein R¹⁴ and Z are as defined in claim 1 and M represents an organo-tin or organo boronic acid group; or
(c) when W represents —S— and Z represents a single bond or —CH₂—, reacting a compound of formula (IV) wherein Hal represents a halogen atom and X, R¹, R³, R⁴, R⁵ and R⁶ are as defined in claim 1,
with a thiol R¹⁴-Z-S—H wherein R¹⁴ and Z are as defined in claim 1 in the presence of a copper (I) salt; or
(d) when W represents —S— and Z represents a single bond or —CH₂—, reacting a compound of formula (V) 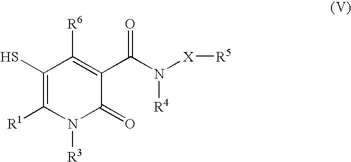
wherein X, R¹, R³, R⁴, R⁵ and R⁶ are as defined in claim 1,
with an electrophile R⁴-Z-L² wherein L² represents a leaving group and R¹⁴ and Z are as defined in claim 1; or
(e) when W represents —SO₂— and Z represents —NR²⁵—, reacting a compound of formula (VI) 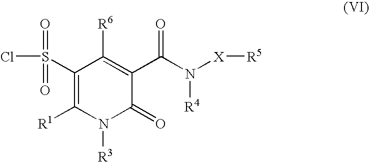
wherein X, R¹, R³, R⁴, R⁵ and R⁶ are as defined in claim 1,
with an amine R¹⁴—NHR²⁵ wherein R¹⁴ and R²⁵ are as defined in claim 1; or
(f) when W represents a sulfinyl (—S(O)—) or a sulfonyl (—S(O)₂—) group, oxidising the corresponding compound wherein W represents a thio (—S—) group;
and optionally after (a), (b), (c), (d), (e) or (f) carrying out one or more of the following:
converting the compound obtained to a further compound of the invention
forming a pharmaceutically acceptable salt of the compound.

9. A pharmaceutical composition comprising a compound of formula (I) or a pharmaceutically acceptable salt thereof as claimed in any one of claims 1 to 7 in association with a pharmaceutically acceptable adjuvant, diluent or carrier.

10. A process for the preparation of a pharmaceutical composition as claimed in claim 9 which comprises mixing a compound of formula (I) or a pharmaceutically acceptable salt thereof as claimed in any one of claims 1 to 7 with a pharmaceutically acceptable adjuvant, diluent or carrier.

11-13. (canceled)

14. A method of treating, or reducing the risk of, a disease or condition in which inhibition of neutrophil elastase activity is beneficial which comprises administering to a patient in need thereof a therapeutically effective amount of a compound of formula (I) or a pharmaceutically acceptable salt thereof as claimed in claim 1.

15. A method of treating, or reducing the risk of, an inflammatory disease or condition which comprises administering to a patient in need thereof a therapeutically effective amount of a compound of formula (I) or a pharmaceutically acceptable salt thereof as claimed in claim 1.

16. The method according to claim 14 or claim 15, wherein the disease or condition is adult respiratory distress syndrome (ARDS), cystic fibrosis, pulmonary emphysema, bronchitis, bronchiectasis, chronic obstructive pulmonary disease (COPD), pulmonary hypertension, asthma, rhinitis, ischemia-reperfusion injury, rheumatoid arthritis, osteoarthritis, cancer, atherosclerosis or gastric mucosal injury.
